SEQUENCE LISTING (14 sequences)

SEQ ID NO: 1 - DNA, 20 bp, Artificial Sequence
  CK2alpha Antisense
gtcccgacatgtcagacagg

SEQ ID NO: 2 - DNA, 21 bp, Artificial Sequence
  CK2alpha Antisense
ccugucugacaugucgggatt

SEQ ID NO: 3 - DNA, 25 bp, Artificial Sequence
  CK2alpah Antisense
atgtcagacaggttggcggacaaag

SEQ ID NO: 4 - DNA, 18 bp, Artificial Sequence
  CK2alpha Antisense
tcactgtatttacctcgg

SEQ ID NO: 5 - DNA, 20 bp, Artificial Sequence
  CK2alpha Antisense
ggacctcctctcaaattctc

SEQ ID NO: 6 - DNA, 20 bp, Artificial Sequence
  CK2alpha Antisense
aggacctttgaagtatcggg

SEQ ID NO: 7 - DNA, 20 bp, Artificial Sequence
  CK2alpha Antisense
tgctccattgcctctcttgc

SEQ ID NO: 8 - DNA, 18 bp, Artificial Sequence
  CK2alpha Antisense
ggcatggcgggcgggacc

SEQ ID NO: 9 - DNA, 20 bp, Artificial Sequence
  CK2alpha Antisense
cgggcatggcgggcgggacc

SEQ ID NO: 10 - DNA, 18 bp, Artificial Sequence
  CK2alpha Antisense
catcttcacgtcagcggc

SEQ ID NO: 11 - DNA, 20 bp, Artificial Sequence
  CK2alpha Antisense
catcttcacgtcagcggctg

SEQ ID NO: 12 - DNA, 1677 bp, homo sapiens
tgtcacccaggctggagtgcagtggcgcaatctcagctcactgcaacctccacctccctggttcaagcgattctcctgcctcctccgcccgacgccccgcgtcccccgccgcgccgccgccgccaccctctgcgccccgcgccgccccccggtcccgcccgccatgcccggcccggccgcgggcagcagggcccgggtctacgccgaggtgaacagtctgaggagccgcgagtactgggactacgaggctcacgtcccgagctggggtaatcaagatgattaccaactggttcgaaaacttggtcggggaaaatatagtgaagtatttgaggccattaatatcaccaacaatgagagagtggttgtaaaaatcctgaagccagtgaagaaaaagaagataaaacgagaggttaagattctggagaaccttcgtggtggaacaaatatcattaagctgattgacactgtaaaggaccccgtgtcaaagacaccagctttggtatttgaatatatcaataatacagattttaagcaactctaccagatcctgacagactttgatatccggttttatatgtatgaactacttaaagctctggattactgccacagcaagggaatcatgcacagggatgtgaaacctcacaatgtcatgatagatcaccaacagaaaaagctgcgactgatagattggggtctggcagaattctatcatcctgctcaggagtacaatgttcgtgtagcctcaaggtacttcaagggaccagagctcctcgtggactatcagatgtatgattatagcttggacatgtggagtttgggctgtatgttagcaagcatgatctttcgaagggaaccattcttccatggacaggacaactatgaccagcttgttcgcattgccaaggttctgggtacagaagaactgtatgggtatctgaagaagtatcacatagacctagatccacacttcaacgatatcctgggacaacattcacggaaacgctgggaaaactttatccatagtgagaacagacaccttgtcagccctgaggccctagatcttctggacaaacttctgcgatacgaccatcaacagagactgactgccaaagaggccatggagcacccatacttctaccctgtggtgaaggagcagtcccagccttgtgcagacaatgctgtgctttccagtggtctcacggcagcacgatgaagactggaaagcgacgggtctgttgcggttctcccacttttccataagcagaacaagaaccaaatcaaacgtcttaacgcgtatagagagatcacgttccgtgagcagacacaaaacggtggcaggtttggcgagcacgaactagaccaagcgaagggcagcccaccaccgtatatcaaacctcacttccgaatgtaaaaggctcacttgcctttggcttcctgttgacttcttcccgacccagaaagcatggggaatgtgaagggtatgcagaatgttgttggttactgttgctccccgagcccctcaactcgtcccgtggccgcctgtttttccagcaaaccacgctaactagctgaccacagactccacagtggggggacgggcgcagtatgtggcatggcggcagttacatattattattttaaaagtatatattattgaataaaaggttttaaaag

SEQ ID NO: 13 - DNA, 1128 bp, Homo Sapiens
gcttctcgttgtgccccgcccgcaagcgccctcctccgggccttcgtgacagccaggtcgtgcgcgggtcatcctgggattggtagttcgctttctctcatttagccagtttctttctctaccggggactccgtgtcccggcatccaccgcggcacctgacccttggcgcttgcgtgttgccctcttccccaccctccctaatttccactccccccaccccacttcgcctgccgcggtcgggtccgcggcctgcgctgtagcggtcgccgccgttccctggaagtagcaacttccctaccccaccccagtcctggtccccgtccagccgctgacgtgaagatgagcagctcagaggaggtgtcctggatttcctggttctgtgggctccgtggcaatgaattcttctgtgaagtggatgaagactacatccaggacaaatttaatcttactggactcaatgagcaggtccctcactaccgacaagctctagacatgatcttggacctggagcctgatgaagaactggaagacaaccccaaccagagtgacctgattgagcaggcagccgagatgctttatggattgatccacgcccgctacatccttaccaaccgtggcatcgcccagatgttggaaaagtaccagcaaggagactttggttactgtcctcgtgtgtactgtgagaaccagccaatgcttcccattggcctttcagacatcccaggtgaagccatggtgaagctctactgccccaagtgcatggatgtgtacacacccaagtcatcaagacaccatcacacggatggcgcctacttcggcactggtttccctcacatgctcttcatggtgcatcccgagtaccggcccaagagacctgccaaccagtttgtgcccaggctctacggtttcaagatccatccgatggcctaccagctgcagctccaagccgccagcaacttcaagagcccagtcaagacgattcgctgattccctcccccacctgtcctgcagtctttgacttttcctttcttttttgccaccctttcaggaaccctgtatggtttttagtttaaattaaaggagtcgttattgtggtgggaatatgaaataaagtagaagaaaaggcc

SEQ ID NO: 14 - DNA, 2323 bp, Homo Sapiens
cccgcctcctggtaggagggggtttccgcttccggcagcagcggctgcagcctcgctctggtccctgcggctggcggccgagccgtgtgtctcctcctccatcgccgccatattgtctgtgtgagcagaggggagagcggccgccgccgctgccgcttccaccacagtttgaagaaaacaggtctgaaacaaggtcttacccccagctgcttctgaacacagtgactgccagatctccaaacatcaagtccagctttgtccgccaacctgtctgacatgtcgggacccgtgccaagcagggccagagtttacacagatgttaatacacacagacctcgagaatactgggattacgagtcacatgtggtggaatggggaaatcaagatgactaccagctggttcgaaaattaggccgaggtaaatacagtgaagtatttgaagccatcaacatcacaaataatgaaaaagttgttgttaaaattctcaagccagtaaaaaagaagaaaattaagcgtgaaataaagattttggagaatttgagaggaggtcccaacatcatcacactggcagacattgtaaaagaccctgtgtcacgaacccccgccttggtttttgaacacgtaaacaacacagacttcaagcaattgtaccagacgttaacagactatgatattcgattttacatgtatgagattctgaaggccctggattattgtcacagcatgggaattatgcacagagatgtcaagccccataatgtcatgattgatcatgagcacagaaagctacgactaatagactggggtttggctgagttttatcatcctggccaagaatataatgtccgagttgcttcccgatacttcaaaggtcctgagctacttgtagactatcagatgtacgattatagtttggatatgtggagtttgggttgtatgctggcaagtatgatctttcggaaggagccatttttccatggacatgacaattatgatcagttggtgaggatagccaaggttctggggacagaagatttatatgactatattgacaaatacaacattgaattagatccacgtttcaatgatatcttgggcagacactctcgaaagcgatgggaacgctttgtccacagtgaaaatcagcaccttgtcagccctgaggccttggatttcctggacaaactgctgcgatatgaccaccagtcacggcttactgcaagagaggcaatggagcacccctatttctacactgttgtgaaggaccaggctcgaatgggttcatctagcatgccagggggcagtacgcccgtcagcagcgccaatatgatgtcagggatttcttcagtgccaaccccttcaccccttggacctctggcaggctcaccagtgattgctgctgccaacccccttgggatgcctgttccagctgccgctggcgctcagcagtaacggccctatctgtctcctgatgcctgagcagaggtgggggagtccaccctctccttgatgcagcttgcgcctggcggggaggggtgaaacacttcagaagcaccgtgtctgaaccgttgcttgtggatttatagtagttcagtcataaaaaaaaaattataataggctgattttcttttttctttttttttttaactcgaacttttcataactcaggggattccctgaaaaattacctgcaggtggaatatttcatggacaaatttttttttctcccctcccaaatttagttcctcatcacaaaagaacaaagataaaccagcctcaatcccggctgctgcatttaggtggagacttcttcccattcccaccattgttcctccaccgtcccacactttagggggttggtatctcgtgctcttctccagagattacaaaaatgtagcttctcaggggaggcaggaagaaaggaaggaaggaaagaaggaagggaggacccaatctataggagcagtggactgcttgctggtcgcttacatcactttactccataagcgcttcagtggggttatcctagtggctcttgtggaagtgtgtcttagttacatcaagatgttgaaaatctacccaaaatgcagacagatactaaaaacttctgttcagtaagaatcatgtcttactgatctaaccctaaatccaactcatttatacttttatttttagttcagtttaaaatgttgataccttccctcccaggctccttaccttggtcttttccctgttcatctcccaacatgctgtgctccatagctggtaggagagggaaggcaaaatctttcttagttttctttgtcttggccattttgaattc

(19) United States
(12) Patent Application Publication
(10) Pub. No.: US 2006/0018826 A1
(43) Pub. Date: Jan. 26, 2006

(54) Biologic modulations with nanoparticles

(76) Inventors: Gretchen M. Unger, Chaska, MN (US); Roderic M.K. Dale, Wilsonville, OR (US); Thomas L. Thompson, West Linn, OR (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 10/958,999

(22) Filed: Oct. 5, 2004

Related U.S. Application Data
(63) Continuation of application No. PCT/US03/10729, filed on Apr. 8, 2003.
(60) Provisional application No. 60/394,315, filed on Jul. 8, 2002.
(60) Provisional application No. 60/370,882, filed on Apr. 8, 2002.
(60) Provisional application No. 60/428,296, filed on Nov. 22, 2002.

(51) Int. Cl. A61K 51/00; A61K 48/00; A61K 31/675; C07H 21/04

(52) U.S. Cl. 424/1.11; 514/44; 514/81; 536/23.1; 544/81

(57) ABSTRACT

Certain aspects of the invention relate to the use of small particles in biological systems, including the delivery of biologically active agents to cells or tissues using nanoparticles of less than about 200 nm in approximate diameter. Embodiments include collection of particles having a bioactive component, a surfactant molecule, a biocompatible polymer, and a cell recognition component, wherein the cell recognition component has a binding affinity for a cell recognition target. Compositions and methods of use are also set forth, including the use of antisense directed against Protein Kinase CK2, CK2alpha, CK2 alpha′, and CK2 beta.

### RELATED APPLICATIONS

This application claims priority to U.S. Patent Application Ser. Nos. 60/394,315, filed Jul. 8, 2002; 60/370,882 filed Apr. 8, 2002; 60/428,296, filed Nov. 22, 2002 and Ser. No. 10/378,044 filed on Feb. 28, 2003; which are hereby incorporated herein by reference.

### FIELD OF THE INVENTION

The field of the invention relates to the use of small particles in biological systems, including the delivery of biologically active agents.

### BACKGROUND

Over the past several decades, active and extensive research into the use of small particles in the delivery of therapeutic macromolecules has generated a number of conventional approaches in the preparation of small particles. These approaches typically include the use of heat, high pressure homogenization, or high intensity ultrasound sonication to prepare particles having a diameter of more than 100 nanometers, or high amounts of solvents or oils, cytotoxic chemicals, such as cross-linking agents, catalysts to prepare small particles. These approaches are challenging due to a number of variables.

For example, when organic solvents are included in the manufacturing process for small particles, the organic solvent may denature the therapeutic macromolecule which reduces most, if not all, efficacy of the therapeutic macromolecule. In fact, denaturation of the therapeutic macromolecule may even promote a toxic response upon administration of the small particle.

In addition, when an organic solvent is used to prepare small particles, the organic solvent or solvent soluble polymer may undergo degradation or other reactions that destroys the efficacy of the therapeutic macromolecule. Therefore, organic solvents may generally denature the therapeutic macromolecule during or after preparation of an small particle. As a result, organic solvents are typically removed during the manufacturing process of small particles. However, inclusion of one or more organic solvent removal techniques generally increases the costs and complexity of forming small particles. Additionally, high pressure homogenization or high intensity ultrasound sonication techniques often require complex and expensive equipment that generally increases costs in preparing small particles.

Therapeutic macromolecules also have limited ability to cross cell membranes. Consequently, the future success of antisense and other new molecular approaches requires innovation in drug delivery methods. Delivery of therapeutic macromolecules, particularly nucleic acids, is complicated not only by their size, but also by their sensitivity to omnipresent nuclease activity in vivo.

Therefore, there is a need for methods to prepare small particles without the use of cytotoxic chemicals or complex and expensive equipment. Additionally, a need exists to develop a small particle that may more effectively deliver antisense molecules.

One medical area that would benefit from improved small particle delivery systems is cancer treatment. Much has been already said about the grim survival statistics of head neck cancer in the U.S. and throughout the world (U.S. annual incidence: 40,000; world: 500,000). Following initial treatment with some combination of surgery, radiation and chemotherapy, approximately 20-30% of the head neck cancers diagnosed in the U.S. recur within 5 years. Approximately 50-70% of these tumors recur locally in the head neck region. Of these recurrent tumors, 5 year survival rates linger at approximately 30%. These low survival rates have not improved over the last 15 years and suggest significant opportunity exists to improve the treatment of locally recurring head neck tumors.

### SUMMARY

Included herein are embodiments for making and using nanoparticles that overcome these problems. Cells may take up these nanoparticles through caveolae, which are cholesterol rich vesicles that are smaller than clathrin coated pits and bypass the endosomal pathways. Entrance through caveolae is through 20-60 nanometer openings located on the surface of the target cell. Accordingly, nanoparticles are provided herein that are dimensioned to pass through caveloae, so that the nanoparticle contents are not degraded. Moreover, the nanoparticles are localized to cell nuclei after their introduction into the cell so that the nanoparticle contents are delivered in a highly effective manner that requires lower doses and concentrations than would otherwise be necessary, see copending U.S. patent application Ser. No. 09/796,575, filed Feb. 28, 2001.

Embodiments include methods and compositions for specific delivery of macromolecules and small molecules to cell and tissue-specific targets using ligand-based nanoparticles. Embodiments include nanoparticles that may be assembled from simple mixtures of components comprising at least one ligand for a target cell surface receptor. Nanoparticles may be designed to be metastable, and/or controlled-release forms, enabling eventual release of capsule or particle contents. In one embodiment, particles are manufactured to be smaller than 50 nm enabling efficient cellular uptake by caveolar potocytosis. These particles are further distinguished by their capacity for penetration across tissue boundaries, such as the epidermis and endothelial lumen. In another embodiment, particles are manufactured to be larger than 50 nm, enabling a period of extracellular dissolution. This combined approach of using readily-assembled particles with ligand-based targeting enables a method of rational design for drug delivery based on cell biology and regional administration.

Aspects of the invention relate to the use of small particles in biological systems, including the delivery of biologically active agents using nanoparticles of less than about 200 nm in approximate diameter. Embodiments include collection of particles having a bioactive component, a surfactant molecule, a biocompatible polymer, and a cell recognition component, wherein the cell recognition component has a binding affinity for a cell recognition target. Compositions and methods of use are also set forth.

An embodiment is a collection of particles having a bioactive component, a surfactant molecule having an HLB value of less than about 6.0 units, a biocompatible polymer, and a cell recognition component, wherein the collection of particles has an average diameter of less than about 200 nanometers as measured by atomic force microscopy following drying of the collection of particles. The cell recognition component may have a binding affinity for a cell recognition target. The target may be a member of the group consisting of cell adhesion molecules, immunoglobulin superfamily, cell adhesion molecules, integrins, cadherins, selecting, growth factor receptors, collagen receptors, laminin receptors, fibronectin receptors, chondroitin sulfate receptors, dermatan sulfate receptors, heparin sulfate receptors, keratan sulfate receptors, elastin receptors, and vitronectin receptors. Additional embodiments have a cell recognition component that is a ligand that has an affinity for the cell recognition target and the cell recognition target is a member of the group consisting of immunoglobulin superfamily, cell adhesion molecules, integrins, cadherins, and selectins.

Another embodiment is a collection of particles comprising a bioactive component, a surfactant molecule having an HLB value of less than about 6.0 units, and a biocompatible polymer, wherein the collection of particles has an average diameter of less than about 200 nanometers as measured by atomic force microscopy of a plurality of the particles following drying of the particles. The bioactive component may include, for example, anthracyclines, doxorubicin, vincristine, cyclophosphamide, topotecan, paclitaxel, modulators of apoptosis, and/or growth factors.

Another embodiment is a collection of particles comprising a bioactive component, a surfactant molecule having an HLB value of less than about 6.0 units, and a biocompatible polymer, wherein the particle has an average diameter of less than about 200 nanometers as measured by atomic force microscopy of a plurality of the particles following drying of the particles, and wherein the bioactive component is an antisense polynucleic acid effective to inhibit expression of CK2 polypeptides.

Another embodiment is a method of providing a collection of particles that have a bioactive component, a surfactant having an HLB value of less than about 6.0 units, a biocompatible polymer, and a cell recognition component. The particle collection may have an average diameter of less than about 200 nanometers as measured by atomic force microscopy of a plurality of the particles following drying of the particles. The cell recognition component may have a binding affinity for a member of the group consisting of cell adhesion molecules, immunoglobulin superfamily, cell adhesion molecules, integrins, cadherins, selectins, growth factor receptors, collagen, laminin, fibronectin, chondroitin sulfate, dermatan sulfate, heparin sulfate, keratan sulfate, elastin, and vitronectin.

In some aspects, the invention pertains to an antisense polynucleic acid comprising a sequence, wherein the antisense polynucleic acid suppresses the expression of a polypeptide encoded by a polynucleic acid sequence for the polypeptide chosen from the group consisting of SEQ ID NO 12 SEQ ID NO 13 and SEQ ID NO 14. The antisense polynucleic acid comprises a backbone that has at least two members of the group consisting of unmodified DNA/RNA, DNA/RNA with modified internucleoside linkages, 2′ modified RNA, p-ethoxy-2′omethyl RNA modification, 3′ end-blocked RNA, and 5′ end-blocked RNA.

In additional aspects, the invention pertains to a collection of particles comprising an agent, a surfactant molecule having an HLB value of less than about 6.0 units, and a polymer soluble in aqueous solution. The collection of particles has an average diameter of less than about 100 nanometers as measured by atomic force microscopy of a plurality of the particles following drying of the particles. The agent comprises an antisense polynucleic acid that comprises a sequence, wherein the antisense polynucleic acid suppresses the expression of at least one member of the group consisting of protein kinase CK2, protein kinase CK2 alpha, and protein kinase CK2 beta. Also, the antisense polynucleic acid comprises a backbone that has at least two members of the group consisting of unmodified DNA/RNA, DNA/RNA modified internucleoside linkages, 2′ modified RNA, p-ethoxy-2′omethyl RNA modification, 3′ end-blocked RNA, and 5′ end-blocked RNA.

In further aspects, the invention pertains to a collection of particles comprising a bioactive component, a surfactant molecule having an HLB value of less than about 6.0 units, and a biocompatible polymer. The particle has an average diameter of less than about 200 nanometers as measured by atomic force microscopy of a plurality of the particles following drying of the particles. The bioactive component comprises an antisense polynucleic acid effective to inhibit expression of at least one member of the group consisting of protein kinase CK2, protein kinase CK2 alpha, and protein kinase CK2 beta. Also, the antisense comprises a backbone having at least two members of the group consisting of unmodified DNA/RNA, DNA/RNA with modified internucleoside linkages, 2′ modified RNA, p-ethoxy-2′omethyl RNA modification, 3′ end-blocked RNA, and 5′ end-blocked RNA.

In addition, the invention pertains to a method of delivering a bioactive component to a cell or tissue comprising providing a collection of particles comprising an antisense molecule, a surfactant having an HLB value of less than about 6.0 units, and a biocompatible polymer. The particle has an average diameter of less than about 200 nanometers as measured by atomic force microscopy of a plurality of the particles following drying of the particles. In these embodiments, the bioactive component comprises an antisense polynucleic acid effective to inhibit expression of at least one member of the group consisting of protein kinase CK2, protein kinase CK2 alpha, and protein kinase CK2 beta. The antisense comprises a backbone having at least two members of the group consisting of unmodified DNA/RNA, DNA/RNA with modified internucleoside linkages, 2′ modified RNA, p-ethoxy-2′omethyl RNA modification, 3′ end-blocked RNA, and 5′ end-blocked RNA.

Furthermore, the invention pertains to a method of delivering an anti-cancer agent to cancer cells, the method comprising contacting the cancer cells with a collection of particles. The particles comprise the anticancer agent, a surfactant having an HLB value less than about 6.0 units, and a biocompatible polymer. The anticancer agent comprises an antisense polynucleic acid effective to inhibit expression of at least one member of the group consisting of protein kinase CK2, protein kinase CK2 alpha, and protein kinase CK2 beta. The antisense comprises a backbone having at least two members of the group consisting of unmodified DNA/RNA, DNA/RNA with modified internucleoside linkages, 2′ modified RNA, p-ethoxy-2′omethyl RNA modification, 3′ end-blocked RNA, and 5′ end-blocked RNA.

### BRIEF DESCRIPTION OF THE FIGURES

FIG. 1A is a montage of photomicrographs showing nanoparticle uptake in irradiated versus nonirradiated tissues;

FIG. 1B is a montage of photomicrographs showing delivery of macromolecules to peripheral smooth muscle cells after delivery to an arterial lumen;

FIG. 2A is a montage of photomicrographs showing cell-specific targeting using nanoparticles comprising fibronectin or tenascin;

FIG. 2B is a montage of photomicrographs showing nanoparticles comprising fibronectin delivered to an arterial lumen penetrate through the arterial walls;

FIG. 2C is a montage of photomicrographs showing astrocytic uptake and delivery of bioactive agents using nanoparticles comprising FN;

FIG. 2D is a montage of photomicrographs showing delivery of agents to cells in suspension using nanoparticles comprising various ligands for targeting specific cell types;

FIG. 3A is a montage of photomicrographs showing delivery of nanoparticle contents to cells;

FIG. 3B is a montage of photomicrographs showing targeted delivery to cells mediated by cell surface receptor binding events;

FIG. 3C is a montage of photomicrographs showing nanoparticles made with hydrophilic and hydrophobic peptides;

FIG. 3D is a is a montage of photomicrographs showing keratinocytes treated with nanoparticles having FITC-dextran;

FIG. 4A is a montage of photomicrographs showing nanoparticles of various sizes comprising plasmids;

FIG. 5A is a graph showing a comparison of both nanoparticle and liposomal delivery of antisense molecules;

FIG. 5B is a graph showing cellular dose response curves for CK2α antisense sequences;

FIG. 5C is a graph showing cellular dose response curves for nanoparticles comprising a small molecule toxin or a CK2α antisense sequence;

FIG. 5D is a graph showing cellular dose response curves for nanoparticles comprising various agents for targeting prostate cancer cells;

FIGS. 6A and 6B are montages of photomicrographs that show delivery of anti-tumor compounds using nanoparticles;

FIG. 7 is a graph, with a photographic inset, that shows the treatment of cancer in animals using nanoparticles having CK2α antisense sequences;

FIG. 8 is a montage of photomicrographs showing the use of nanoparticles to deliver CK2α to modulate cell proliferation;

FIG. 9 is a listing of the mRNA sequence for Protein Kinase CK2 alpha prime;

FIG. 10 is a listing of the mRNA sequence for Protein Kinase CK2 beta;

FIG. 11 is a listing of the mRNA sequence for Protein Kinase CK2 alpha.

### DETAILED DESCRIPTION

Embodiments are described herein for making and using nanoparticles that effectively deliver therapeutic compositions, including, for example, macromolecules. Without being limited to a particular theory of action, certain embodiments of the nanoparticles are sized so as to enter through cellular caveolae and thereby overcome many of the limitations of conventional therapies. The nanoparticles enter the cell release agents that modulate cellular activity. Examples of agents are toxins, genes, and antisense DNA molecules. Other embodiments are nanoparticles that have agents for visualizing the cell, e.g., fluorescent markers or dye. Other embodiments are particles that target the exterior of a cell, or areas outside of a cell and subsequently are taken up by cells or subsequently release agents. Other embodiments are controlled release systems for controllably releasing nanoparticles for sustained delivery of the nanoparticles and agents associated with the nanoparticles. Further, methods for targeting specific cells and treating certain conditions using therapeutics delivered with nanoparticles are set forth.

Detailed methods for making such nanoparticles are set forth in commonly owned copending U.S. patent application Ser. No. 09/796,575, filed Feb. 28, 2001. Additionally, detailed methods of making alternative forms of nanoparticles are presented herein, as well as methods of making and using the same. Certain embodiments address useful recipes for making nanoparticles, as well as therapeutic molecules for use with the same. Although the term nanoparticle is adopted herein to describe certain preferred embodiments for particles, the term includes nanoparticles and nanospheres. In general, a nanoparticle is a particle that is less than about 100 nm in average diameter, but other sizes and conformations of the nanoparticles are also contemplated.

Since nanoparticles are described herein may be capable of caveolaer cell entry, they are effective vehicles for delivering agents to cells in circumstances where conventional particles are not effective, including microparticles, liposomes, stealth liposomes, and other conventionally known particulate delivery systems, including those that have referred to as nanoparticles by others. As set forth below, nanoparticles are generally small relative to conventional particles so that delivery through the blood system and tissue is enhanced relative to conventional particle technology. The nanoparticles are generally useful for therapeutic applications, research applications, and applications in vivo, ex vivo, and in vitro.

Nanoparticles may be sized, as described herein, to enter cells via cellular caveloae, which are cholesterol-rich structures present in most cells and cell types. Entrance to these vesicles is through 20-60 nm openings. Caveolae a.k.a. plasmalemmel vesicles are small (50-80 nm), cholesterol-rich vesicles which likely derive from mobile microdomains of cholesterol in the cell membrane, a.k.a lipid rafts. These vesicles participate in a receptor-mediated uptake process known as potocytosis. Because of the lipid nature of caveolae, receptors that populate or traffic to caveolae following ligand binding typically include receptors with fatty acid tails such as GPI-linked or integrin receptors. An integral role for caveolin in mediating 13-1 integrin signaling and maintenance of focal adhesions has been documented.

In contrast, the delivery of larger objects to cells is conventionally attempted using other pathways. These pathways vary in the size of molecules that they can accept. The coated pit pathway is best-known and well-studied as the pathway for receptor-mediated endocytosis. Coated pits evolve into endosomes coated with clathrin that are typically in the range of 150-200 nm. Unless a specific sorting event occurs, endosomes constitutively deliver their contents to a lysosomal vesicle for degradation (reviewed in Mukerjee, 1997).

Nanoparticles and Methods of Making

The manufacture and process chemistry of nanoparticles is described in detail in U.S. patent Ser. No. 09/796,575 filed Feb. 28, 2001. In brief, a suitable method of making a nanoparticle is to form a dispersion of micelles by forming a plurality of surfactant micelles, wherein the plurality of surfactant micelles comprises a surfactant interfacing with a bioactive component, wherein the surfactant can have a hydrophile-lipophile-balance (HLB) value of less than about 6.0 units. Then the surfactant micelles are dispersed into an aqueous composition, wherein the aqueous composition comprises a hydrophilic polymer so that the hydrophilic polymer associates with the surfactant micelles to form stabilized surfactant micelles. The stabilized micelles may have an average diameter of less than about 200 or 100 or 50 nanometers. Non-ionic surfactants may alternatively be used. The stabilized surfactant micelles may be precipitated, e.g. using a cation, to form nanoparticles having an average diameter of less than about 200 or 100 or 50 nanometers, as measured by atomic force microscopy of the particles following drying of the particles. Moreover, in some embodiments, the particles may be incubated in the presence of at least one cation. Embodiments wherein nanoparticles have a diameter of less than 200 or 100 or 50 nm, including all values within the range of 5-200 nm, are contemplated. Following incubation, particles are collected by centrifugation for final processing. Particles show excellent freeze-thaw stability, stability at −4° C., mechanical stability and tolerate speed-vacuum lyophilization. Stability is measured by retention of particle size distribution and biological activity. Drug stocks of 4 mg/ml are routinely produced with 70-100% yields.

The term precipitate refers to a solidifying or a hardening of the biocompatible polymer component that surrounds the stabilized surfactant micelles. Precipitation also encompasses crystallization of the biocompatible polymer that may occur when the biocompatible polymer component is exposed to the solute. Examples of cations for precipitation include, for example, Mn2+, Mg2+, Ca2+, A13+, Be2+, Li⁺, Ba2+, Gd3+.

The amount of the surfactant composition in some embodiments may range up to about 10.0 weight percent, based upon the weight of a total volume of the stabilized surfactant micelles. Typically however, the amount of the surfactant composition is less than about 0.5 weight percent, and may be present at an amount of less than about 0.05 weight percent, based upon the total weight of the total volume of the stabilized surfactant micelles. A person of ordinary skill in the art will recognize that all possible ranges within the explicit ranges are also contemplated.

A nanoparticle may be a physical structure such as a particle, nanocapsule, nanocore, or nanosphere. A nanosphere is a particle having a solid spherical-type structure with a size of less than about 1,000 nanometers. A nanocore refers to a particle having a solid core with a size of less than about 1,000 nanometers. A nanocapsule refers to a particle having a hollow core that is surrounded by a shell, such that the particle has a size of less than about 1,000 nanometers. When a nanocapsule includes a therapeutic macromolecule, the therapeutic macromolecule is located in the core that is surrounded by the shell of the nanocapsule.

Embodiments herein are described in terms of nanoparticles but are also contemplated as being performed using nanocapsules, the making and use of which are also taught in commonly assigned copending application Ser. No. 09/796,575, filed Feb. 28, 2001, which teaches methods for making particles having various sizes, including less than about 200 nm, from about 5-200 nm, and all ranges in the bounds of about 5 and about 200 nm. The same application teaches how to make s50 nanoparticles. An s50 nanoparticle is a nanoparticle that has an approximate diameter of less than about 50 nm.

The bioactive component, in some embodiments, may be partitioned from the hydrophilic polymer in the nanoparticles, and may be, for example, hydrophobic or hydrophilic. Bioactive components may include proteins, peptides, polysaccharides, and small molecules, e.g., small molecule drugs. Nucleic acids are also suitable bioactive components for use in nanoparticles, including DNA, RNA, mRNA, and including antisense RNA or DNA. When nucleic acids are the bioactive component, it is usually desirable to include a step of condensing the nucleic acids with a condensation agent prior to coating or complexing the bioactive component with the surfactant, as previously set forth in U.S. patent application Ser. No. 09/796,575, filed Feb. 28, 2001.

A wide variety of polymers may be used as the biocompatible polymer, including many biologically compatible, water-soluble and water dispersible, cationic or anionic polymers. Due to an absence of water diffusion barriers, favorable initial biodistribution and multivalent site-binding properties, hydrophilic polymer components are typically useful for enhancing nanoparticle distribution in tissues. However, it will be apparent to those skilled in the art that amphoteric and hydrophobic polymer components may also be used as needed. The biocompatible polymer component may be supplied as individual biocompatible polymers or supplied in various prepared mixtures of two or more biocompatible polymers that are subsequently combined to form the biocompatible polymer component. Though descriptions of the present invention are primarily made in terms of a hydrophilic biocompatible polymer component, it is to be understood that any other biocompatible polymer, such as hydrophobic biocompatible polymers may be substituted in place of the hydrophilic biocompatible polymer, in accordance with the present invention, while still realizing benefits of the present invention. Likewise, it is to be understood that any combination of any biocompatible polymer may be included in accordance with the present invention, while still realizing benefits of the present invention.

Antisense Molecules and Condensation

Antisense molecules are useful bioactive agents to deliver with nanoparticles. Nanoparticles comprising antisense molecules are typically made with a condensing agent. Some suitable nucleic acid condensing agents are poly(ethylenimine) (PEI) (at a 27,000 MW, PEI was used at about 90% charge neutralization). Polylysine (PLL) (at 7,000-150,000 molecular weight. PLL condensing materials were conjugated with nuclear signal localization peptides, e.g., SV-40 T using carbodiimide chemistry available from Pierce Chemical (Rockford, Ill.). Preparations of nuclear matrix proteins (NMP). NMP were collected from a rat fibroblast cell line, and a human keratinocyte cell line using a procedure described in Gerner et al. J. Cell. Biochem. 71 (1998): 363-374. Protein preparations were conjugated with nuclear signal localization peptides as described.

Additional materials for use as condensation components are spermine, polyornithine, polyarginine, spermidine, VP22 protein constructs, block and graft copolymers of N-(2-hydroxypropyl)methacrylamide (HPMA) with 2-(trimethylammonio)ethyl methacrylate (TMAEM),poly[2-(dimethylamino)ethyl methacrylate], p(DMAEMA), Protamine, sulfate, and peptide constructs derived from histones. Additional condensation components are know, for example as in U.S. Pat. No. 6,153,729. Antisense molecules typically require a relatively smaller condensation agent than relatively larger nucleic acid molecules. Targeting agents may also be conjugated to condensation agents, e.g., as in U.S. Pat. No. 5,922,859 and PCT Application WO/01 089579.

Targeting Components

Nanoparticles can comprise various targeting components, e.g., ligands, to target the nanoparticle and its contents to, e.g., specific cells. The contents of the nanoparticle may be, for example, therapeutic agents that alter the activity of the cell, or a marker. The ligands can be in coatings and/or otherwise incorporated into the nanoparticles. For example, if one more than one type of cell is being cultured, a particular cell type or subset of cells may be targeted using nanoparticles having ligands that are specific to particular targets on the cells. Thus, for example, several cells in the field of view of a microscope may be observed while a subset of the cells are undergoing treatment. Thus some of the cells serve as controls for the treated cells. Or, cells may advantageously be treated while cultured with other cells, for example, some cultured stem cells are known to be advantageously grown in co-culture with other cell types. Table 1 sets forth some ligands. A ligand is a molecule that specifically binds to another molecule, which may be referred to as a target. Thus a ligand for a growth factor receptor may be, e.g., a growth factor, a fragment of a growth factor, or an antibody. Those of ordinary skill in these arts are able to distinguish specific binding from non-specific binding; for example, the identification of a ligand for a cell receptor requires distinguishing it from other molecules that nonspecifically bind the receptor.

Targeting components and/or agents delivered using nanoparticles may copolymerized, linked to, fused with, or otherwise joined or associated with other molecules, e.g., see Halin et. al, Nature Biotech. (2002) 20:264-69, “Enhancement of the antitumor activity of interleukin-12 by targeted delivery to neovasculature” for a review of fusion proteins.

Moreover, antibodies (described below) or peptides may be developed to target specific tissues. For example, a screening assay may be performed using a library and a target. Thus a library of potential ligands may be screened against targets, e.g., tumor tissue. An example of a screening method is set forth in U.S. Pat. No. 6,232,287, which describes various phage panning methods, both in vitro and in vivo. Such peptides may be incorporated into nanoparticles for targeting uses.

TABLE 1A
Targeting components for particles

| Target cell | Targeting component | Reference/Source |
|---|---|---|
| Endothelial cells | Albumin | U.S. Pat. No. 6,204,054. (for trancytosis) |
| Keratinocytes | Laminin | Glia 8:71 |
| Tumor cells | thrombospondin (TSP) | Wang et. al, Am. J Surg. 170(5) 502-5 |
| | Osteopontin (OP) | Senger et. al, Ann NY Acad Sci 760: 83-100 |
| | Thrombin-cleaved OP | |
| | Fibronectin | Unger et. al, 2001, AAPS Pharmsci 3(3) |
| | | Supplement: 3731 |
| Myocytes | Fibronectin, Laminin | Hornberger, Circ Res. 87(6): 508-15 |
| | β1d integrin ligands | Am. J. Phys. 279(6): H2916-26 |
| | PVP 10,000 MW | |
| hepatocytes/liver | DGEA peptide | Sponsel et. al, Am J. Phys 271: c721-c272 |
| cells | | |
| hepatic stellate | Collagen, laminin | Gastroent 110: 1127-1136 |
| chondrocytes/bone | Osteopontin | Cell Ad Commun 3: 367-374, U.S. Pat. No. 6074609, |
| cells | | U.S. Pat. No. 5770565, PCT W0 0980837A1, PCT |
| | | W0 0209735A2 |
| | BMP | U.S. Pat. No. 6352972 |
| | SPARC/osteonectin | PCT W0072679a1 |
| | collagen2 | PCT W0 145764a1 |
| | HA | U.S. Pat. Nos. 51,283,26 & 5,866,165 |
| | Osteocalcin | U.S. Pat. No. 6,159,467 |
| Smooth muscle cells | Osteopontin | U.S. Pat. No. 5849865 |
| Stem cells | FN, rE-selectin, HA | Kronenwett et. al, Stem Cells 18(5)320-330 |
| Neurons | Nerve Growth Factor, | Development 124(19): 3909-3917 |
| | Agrin | |
| | contactin ligand | U.S. Pat. No. 5766922 |
| | NCAM, L1 | U.S. Pat. No. 5792743 |
| | KAL | U.S. Pat. No. 6121231 |
| | Phosphacan | U.S. Pat. No. 5625040 |
| | Neurocan | U.S. Pat. No. 5648465 |
| | Cytotactin | U.S. Pat. No. S 6482410 |
| | Laminin, KS- and β1k | U.S. Pat. No. 5,610,031 |
| | chain | U.S. Pat. No. 5,580,960 |
| | Merosin | U.S. Pat. No. 5,872,231 |
| Schwann | Ninjurin | U.S. Pat. No. 6,140,117 |
| cells/neuron | | |
| Retinal ganglion | Osteonectin | J. Histochem Chem 46(1): 3-10 |
| | Laminin | Dev. Biol. 138: 82-93 |
| Muller cells | rNcam, r L-1 rN-cadherin | Dev. Biol. 138(1): 82-93 |
| Blood-Brain barrier | Peptide vectors e.g. d- | Rouselle et. al, Molecular Pharmacology, |
| | penetratin, pegelin, | (2000) 57: 679-686 |
| | protegrins and related | |

TABLE 1B
Additional Candidate Excipients for angiogenic and anti-tumor particle targeting
agents

| | Potential Role in Tumor | |
|---|---|---|
| Candidate Particle Material | Biology | Reference |
| Recombinant Pex binding | Extravasation of tumor cells | Bello et. al, Cancer Research |
| domain of membrane- | from bloodstream into distant | (2001) 61: 8730-36 |
| associated Matrix | site from primary tumor | |
| Metalloproteinase-1 | | |
| Bovine bone-derived | Chemokine attracting | Jacob et. al, Cancer Research |
| Osteonectin | metastatic tumor cells to bone | (1999) 59: 4453-57 |
| Fibronectin inhibitory | Blocks α₅β₁ integrin binding | Livant et. al, Cancer |
| peptide, PHSCN | site on migrating tumor cells, | Research (2000) 60: 309- |
| | preventing tissue | |
| | extravasation | |
| Recombinant truncated | Modified ligand for CEA | PCT WO 02100343A2 |
| Galectin-3 | antigen, plays role in tumor | Glinsky et. al, Cancer |
| | cell extravasation | Research (2001) 61: 4851-57 |
| Hyaluronan | Feature of tumor stroma, | Simpson et. al, J Biol. Chem |
| | plays role in tumor | (2001) 276(21): 17949-57 |
| | extravasation | |
| Tenascin | Feature of tumor stroma | Tuxhorn et. al, J Urol. (2001) |
| | | 166: 2472-2483 |

Cellular Adhesion Molecules

Embodiments include, e.g., nanoparticles and particles that comprise ligands that bind to cellular adhesion molecules and thereby target the nanoparticle and its contents to specific cells. Various cell surface adhesion molecules are active in numerous cellular processes that include cell growth, differentiation, development, cell movement, cell adhesion, and cancer metastasis. There are at least four major families of cell adhesion molecules: the immunoglobulin (Ig) superfamily, integrins, cadherins, and selecting. Cell adhesion molecules are critical to numerous cellular processes and responses. Additionally, they also play a role in various disease states. For example, tumorigenesis is a process that involves cell adhesion molecules. For successful tumorigenesis, there must be changes in cellular adhesivity which facilitate the disruption of normal tissue structures. Cell adhesion molecules are objects of intense study and improved tools for use with these molecules are required for in vitro and in vivo applications.

Members of the Ig superfamily include the intercellular adhesion molecules (ICAMs), vascular-cell adhesion molecule (VCAM-1), platelet-endothelial-cell adhesion molecule (PECAM-1), and neural-cell adhesion molecule (NCAM). Each Ig superfamily cell adhesion molecule has an extracellular domain, which has several Ig-like intrachain disulfide-bonded loops with conserved cysteine residues, a transmembrane domain, and an intracellular domain that interacts with the cytoskeleton. The Ig superfamily cell adhesion molecules are calcium-independent transmembrane glycoproteins.

Integrins are transmembrane proteins that are constitutively expressed but require activation in order to bind their ligand. Many protein and oligopeptide ligands for integrins are known. Integrins are non-covalently linked heterodimers having alpha (α) and beta (β) subunits. About 15 α subunits and 8 β subunits have been identified. These combine promiscuously to form various types of integrin receptors but some combinations are not available, so that there are subfamilies of integrins that are made of various α and β combinations. Integrins appear to have three activation states: basal avidity, low avidity, and high avidity. Additionally, cells will alter integrin receptor expression depending on activation state, maturity, or lineage.

The cadherins are calcium-dependent adhesion molecules and include neural (N)-cadherin, placental (P)-cadherin, and epithelial (E)-cadherin. All three belong to the classical cadherin subfamily. There are also desmosomal cadherins and proto-cadherins. Cadherins are intimately involved in embryonic development and tissue organization. They exhibit predominantly homophilic adhesion, and the key peptidic motifs for binding have been identified for most cadherins. The extracellular domain consists of several cadherin repeats, each is capable of binding a calcium ion. Following the transmembrane domain, the intracellular domain is highly conserved. When calcium is bound, the extracellular domain has a rigid, rod-like structure. The intracellular domain is capable of binding the a, b, and g catenins. The adhesive properties of the cadherins have been shown to be dependent upon the ability of the intracellular domain to interact with cytoplasmic proteins such as the catenins.

The selectins are a family of divalent cation dependent glycoproteins that bind carbohydrates, binding fucosylated carbohydrates, especially, sialylated Lewisx, and mucins. The three family members include: Endothelial (E)-selectin, leukocyte (L)-selectin, and platelet (P)-selectin. The extracellular domain of each has a carbohydrate recognition motif, an epidermal growth factor (EGF)-like motif, and varying numbers of a short repeated domain related to complement-regulatory proteins (CRP). Each has a short cytoplasmic domain. The selectins play an important role in aspects of cell adhesion, movement, and migration.

TABLE 2
Examples of Cell Recognition Components Specific for Cell Recognition Targets

| | Alternative | | |
|---|---|---|---|
| Targeting | Names | | Example of Tumor |
| Ligands | (trade name) | Target | Target |
| RGD peptide | | Cellular adhesion | Vasculature endothelial |
| | | molecules, such as ανβ3- | cells in solid tumors |
| | | integrin | |
| NGR | | Aminopeptidase N | Vasculature endothelial |
| | | (CD13) | cells in solid tumors |
| Folate | | Folate receptor | Cancer cells that |
| | | | overexpress the folate |
| | | | receptor |
| Transferrin | | Transferrin receptor | Cancer cells that |
| | | | overexpress the |
| | | | transferrin receptor |
| GM-CSF | | GM-CSF receptor | Leukaemic blasts |
| Galactosamine | | Galactosamine receptors | Hepatoma |
| | | on hepatocytes | |
| Anti-VEGFR | 2C3 | Vasculature endothelial | Vasculature endothelial |
| antibody | | growth-factor receptor | cells in solid tumors |
| | | (FLK1) | |
| Anti-ERBB2 | Trastuzumab | ERBB2 receptor | Cells that overexpress |
| antibody | (Herceptin) | | the ERBB2 receptor, |
| | | | such as in breast and |
| | | | ovarian cancers. |
| Anti-CD20 | Rituximab | CD20, a B-cell surface | Non-Hodgkin's |
| antibody | (Rituxan), | antigen | lymphoma and other B- |
| | ibritumomab | | cell lymphoproliferative |
| | tiuxetan (Zevalin) | | diseases |
| Anti-CD22 | Epratuzumab, | CD22, a B-cell surface | Non-Hodgkin's |
| antibody | LL2, RFB4 | antigen | lymphoma and other B- |
| | | | cell lymphoproliferative |
| | | | diseases |
| Anti-CD19 | B4, HD37 | CD19, a pan-B-cell | Non-Hodgkin's |
| antibody | | surface epitope | lymphoma and other B- |
| | | | cell lymphoproliferative |
| | | | diseases |
| Anti-CD33 | Gemtuzumab, | CD33, a sialo-adhesion | Acute myeloid leukemia |
| antibody | ozogamicin | molecule, leukocyte | |
| | (Mylotarg) | differentiation antigen | |
| Anti-CD33 | M195 | CD33, a T-cell epitope | Acute myeloid leukemia |
| Anti-CD25 | Anti-Tac, LMB2 | CD25, α-subunit of the | Hairy-cell leukaemia, |
| | | interleukin-2 receptor on | Hodgkin's and other |
| | | activated T cells | CD25⁺ lymphoma |
| | | | haematological |
| | | | malignancies |
| Anti-CD25 | Denileukin | Interleukin-2 receptor | Cutaneous T-cell |
| | diftitox (Ontak) | | lymphoma |
| Anti-HLA- | Lym1 | HLA-DR10β subunit | Non-Hodgkin's |
| DR10β | | | lymphoma and other B- |
| | | | cell lymphoproliferative |
| | | | diseases |
| Anti-tenascin | 81C6 | Extracellular-matrix | Glial tumors, breast |
| | | protein overexpressed in | cancer |
| | | many tumors | |
| Anti-CEA | MIN-14, F6, | CEA | Colorectal, small-cell |
| | A5B7 | | lung and ovarian cancers |
| Anti-MUC1 | HMFG1, BrE3 | MUC1, an aberrantly | Breast and bladder |
| | | glycosylated epithelial | cancer |
| | | mucin | |
| Anti-TAG72 | CC49, B72.3 | TAG72, oncofetal antigen | Colorectal, ovarian and |
| | | tumor-associated | breast cancer |
| | | glycoprotein-72 | |

Growth Factors and Growth Factor Receptors

Embodiments include, e.g., nanoparticles associated with growth factors so that the nanoparticles are specifically targeted to cells expressing the growth factor receptors. Other embodiments include nanoparticles having growth factors that are delivered to the cell to modulate the activity of the cell. Other embodiments include ligands that specifically bind to growth factor receptors so as to specifically target the nanoparticle to cells having the growth factor receptor.

Growth factors are active in many aspects of cellular and tissue regulation including proliferation, hyperproliferation, differentiation, trophism, scarring, and healing, as shown in, for example, Table 3. Growth factors specifically bind to cell surface receptors. Many growth factors are quite versatile, stimulating cellular activities in numerous different cell types; while others are specific to a particular cell-type. Targeting nanoparticles to a growth factor receptor enables the activity of the cell to be controlled. Thus many aspects of physiological activity may be controlled or studied, including proliferation, hyperproliferation, and healing. A growth factor refers to a growth factor or molecules comprising an active fragment thereof, and includes purified native polypeptides and recombinant polypeptides.

Nanoparticles may be targeted to growth factor receptors by a variety of means. For example, antibodies against the receptor may be created and used on the nanoparticles for direction specifically to the receptor. Or, the growth factor, or a fragment thereof, may be used on the nanoparticles to directed specifically to the receptor. The blinding of growth factors to growth factor receptors has, in general, been extensively studied, and short polypeptide sequences that are a fragment of the growth factors, and bind to the receptors, are known.

For example, if it is desirable to limit the proliferation of glial or smooth muscle cells, a particle associated with a cell behavior modulating agent, e.g., a toxin or antiproliferative agent, may be decorated with a ligand that specifically binds PDGF-R (Table 3). Since PDGF-R is preferentially expressed by glial or smooth muscle cells, the particles will preferentially be taken up by glial or smooth muscle cells. The toxin would kill the cells or the antiproliferative agent would reduce proliferation. Similarly, other cellular activities, e.g., as set forth in Table 3, may be controlled by specifically targeting nanoparticles having modulating agents.

TABLE 3
Growth Factors and Growth Factor Receptors for Cell and Tissue Targeting

| Factor | Receptor | Source | Activity | Comments |
|---|---|---|---|---|
| PDGF | PDGF-R | platelets, | proliferation of | two different |
| | | endothelial | connective | protein chains |
| | | cells, placenta | tissue, glial and | form 3 distinct |
| | | | smooth muscle | dimer forms; AA, |
| | | | cells | AB and BB |
| EGF | EGF-R | submaxillary | proliferation of | |
| | | gland, Brunners | mesenchymal, | |
| | | gland | glial and | |
| | | | epithelial cells | |
| TGF-a | TGF-a-R | common in | active for normal | related to EGF |
| | | transformed | wound healing | |
| | | cells | | |
| FGF | FGF-R | wide range of | promotes | at least 19 family |
| | | cells; protein is | proliferation of | members, 4 |
| | | associated with | many cells; | distinct receptors |
| | | the ECM | inhibits some | |
| | | | stem cells | |
| NGF | NGF-R | | promotes neurite | related proteins |
| | | | outgrowth and | identified as |
| | | | neural cell | proto-oncogenes; |
| | | | survival | trkA, trkB, trkC |
| Erythropoietin | Erythropoietin-R | kidney | promotes | |
| | | | proliferation and | |
| | | | differentiation of | |
| | | | erythrocytes | |
| TGF-b | TGF-b-R | activated TH₁ | anti- | at least 100 |
| | | cells (T-helper) | inflammatory, | different family |
| | | and natural | promotes wound | members |
| | | killer (NK) cells | healing, inhibits | |
| | | | macrophage and | |
| | | | lymphocyte | |
| | | | proliferation | |
| IGF-I | IGF-I-R | primarily liver | promotes | related to IGF-II |
| | | | proliferation of | and proinsulin, |
| | | | many cell types | also called |
| | | | | Somatomedin C |
| IGF-II | IGF-II-R | variety of cells | promotes | related to IGF-I |
| | | | proliferation of | and proinsulin |
| | | | many cell types | |
| | | | primarily of fetal | |
| | | | origin | |

Epidermal growth factor (EGF), like all growth factors, binds to specific high-affinity, low-capacity cell surface receptors. Intrinsic to the EGF receptor is tyrosine kinase activity, which is activated in response to EGF binding. EGF has a tyrosine kinase domain that phosphorylates the EGF receptor itself (autophosphorylation) as well as other proteins, in signal transduction cascades. Experimental evidence has shown that the Neu proto-oncogene is a homologue of the EGF receptor, indicating that EGF is active in cellular hyperproliferation. EGF has proliferative effects on cells of both mesodermal and ectodermal origin, particularly keratinocytes and fibroblasts. EGF exhibits negative growth effects on certain carcinomas as well as hair follicle cells. Growth-related responses to EGF include the induction of nuclear proto-oncogene expression, such as Fos, Jun and Myc.

Fibroblast Growth Factors (FGFs) are a family of at least 19 distinct members. Kaposi's sarcoma cells (prevalent in patients with AIDS) secrete a homologue of FGF called the K-FGF proto-oncogene. In mice the mammary tumor virus integrates at two predominant sites in the mouse genome identified as Int-1 and Int-2. The protein encoded by the Int-2 locus is a homologue of the FGF family of growth factors. A prominent role for FGFs is in the development of the skeletal system and nervous system in mammals. FGFs also are neurotrophic for cells of both the peripheral and central nervous system. Additionally, several members of the FGF family are potent inducers of mesodermal differentiation in early embryos. The FGFs interact with specific cell-surface receptors that have been identified as having intrinsic tyrosine kinase activity. The Flg proto-oncogene is a homologue of the FGF receptor family. FGFR3 is predominantly expressed in quiescent chondrocytes where it is responsible for restricting chondrocyte proliferation and differentiation. In mice with inactivating mutations in FGFR3 there is an expansion of long bone growth and zones of proliferating cartilage further demonstrating that FGFR3 is necessary to control the rate and amount of chondrocyte growth. Platelet-Derived Growth Factor (PDGF) has two distinct polypeptide chains, A and B. The c-S is proto-oncogene has been shown to be homologous to the PDGF A chain. Like the EGF receptor, the PDGF receptors have autophosphorylating tyrosine kinase activity. Proliferative responses to PDGF action are exerted on many mesenchymal cell types. Other growth-related responses to PDGF include cytoskeletal rearrangement and increased polyphosphoinositol turnover. PDGF induces the expression of a number of nuclear localized proto-oncogenes, such as Fos, Myc and Jun.

Transforming Growth Factors-β (TGFs-β) was originally characterized as a protein (secreted from a tumor cell line) that was capable of inducing a transformed phenotype in non-neoplastic cells in culture, and thus is implicated in numerous hyperproliferation disorders. The TGF-β-related family of proteins includes the activin and inhibin proteins. The Mullerian inhibiting substance (MIS) is also a TGF-β-related protein, as are members of the bone morphogenetic protein (BMP) family of bone growth-regulatory factors. Indeed, the TGF-β family may comprise as many as 100 distinct proteins, all with at least one region of amino-acid sequence homology. There are several classes of cell-surface receptors that bind different TGFs-β with differing affinities. The TGF-β family of receptors all have intrinsic serine/threonine kinase activity and, therefore, induce distinct cascades of signal transduction. TGFs-βs have proliferative effects on many mesenchymal and epithelial cell types and sometimes demonstrate anti-proliferative effects on endothelial cells.

Transforming Growth Factor-a (TGF-α) was first identified as a substance secreted from certain tumor cells that, in conjunction with TGF-β-1, could reversibly transform certain types of normal cells in culture, and thus is implicated in numerous hyperproliferative disorders. TGF-α binds to the EGF receptor, as well as its own distinct receptor, and it is this interaction that is thought to be responsible for the growth factor's effect. The predominant sources of TGF-α are carcinomas, but activated macrophages and keratinocytes (and possibly other epithelial cells) also secrete TGF-α. In normal cell populations, TGF-α is a potent keratinocyte growth factor.

Tumor Necrosis Factor-β (TNF-β) TNF-β (also called lymphotoxin) is characterized by its ability to kill a number of different cell types, as well as the ability to induce terminal differentiation in others. One significant non-proliferative response to TNF-β is an inhibition of lipoprotein lipase present on the surface of vascular endothelial cells. The predominant site of TNF-β synthesis is T-lymphocytes, in particular the special class of T-cells called cytotoxic T-lymphocytes (CTL cells). The induction of TNF-β expression results from elevations in IL-2 as well as the interaction of antigen with T-cell receptors.

Extracellular Matrix Molecules

Embodiments can be particles, e.g., nanoparticles, associated with extracellular matrix molecules so that the particles are specifically targeted to cells expressing receptors for the extracellular matrix molecules. Alternatively, particles may comprise ligands for the extracellular matrix molecules so that the particles become associated with the extracellular matrix molecules on tissues or cells.

The extracellular matrix comprises a variety of proteins and polysaccharides that are assembled into organized matrices that form the scaffold of tissues. The common components of the extracellular matrix can be referred to as extracellular matrix molecules. Examples of extracellular matrix molecules are tenacin, collagen, laminin, fibronectin, hyaluronic acid, chondroitin sulfate, dermatan sulfate, heparin sulfate, heparin, keratan sulfate, elastin, vitronectin, and subtypes thereof. Cells typically secrete extracellular matrix molecules in response to their environments, so that the patterns of extracellular matrix molecule expression may be indicative of certain conditions. For example, EDA, a domain of fibronectin may be targeted for cancer.

Nanoparticles targeted to the extracellular matrix are useful for variety of therapeutic, scientific, and research applications. For example, extracellular matrix molecules specifically bind to receptors on cells, so that nanoparticles comprising extracellular matrix molecules are thereby targeted to extracellular matrix molecule receptors. Further, drugs may be targeted to the extracellular matrix by making nanoparticles having ligands and/or coatings that bind extracellular matrix molecules. Moreover, particles having a visualization agents directed to extracellular matrix molecules may be used for microscopy, e.g. fluorescence or histochemistry.

Aberration in the patterns of expression of extracellular matrix molecules can indicate pathological conditions. For example, human tenascin is an extracellular matrix molecule, a 240.7 kDa glycoprotein. Tenascin is found in abundance in embryonic tissue, whereas the expression in normal adult tissue is limited. Tenascin has been reported to be expressed in the stroma of many tumors, including gliomas, breast, squamous cell and lung carcinomas. Thus it is possible to control hyperproliferative conditions, including many tumors, by specifically directing therapeutic agents to tenacin.

Tenascin is an extracellular matrix molecule that is useful for nanoparticles. Tenascin is a branched, 225 KD fibronectin-like (FN) extracellular protein prominent in specialized embryonic tissues, wound healing and tumors. The appearance of tenascin-C surrounding oral squamous cell carcinomas appears to be a universal feature of these tumors, while tenascin-rich stroma has been consistently observed adjacent to basal cell, esophageal, gastric, hepatic, colonic, glial and pancreatic tumor nests. Production of TN by breast carcinoma cells and stromal fibroblasts correlates with increased invasiveness. In the adult, normal cells aside from wound-activated keratinocytes, do not migrate on tenascin. However, integrin receptors capable of mediating migration on TN by carcinoma cells include αᵥβ₁, αᵥβ₃ and αᵥβ₆. Based on this information, we hypothesized that TN nanoparticles could deliver nucleic acids specifically via receptor-mediated caveolar endocytosis.

Tenascin has been implicated in cancer activities and also as being specific for smooth muscle cells; furthermore, peptidic domains of tenascin have been identified e.g., as in U.S. Pat. No. 6,124,260. Moreover, tenascin peptides and domains for adhesion with particular cell types, as well as functional and structural aspects of tenascin, e.g., Aukhilt et al., J. Biol. Chem., Vol. 268, No. 4, 2542-2553. Moreover, the interaction between smooth muscle cells and tenascin-C has been elucidated. It is believed that the interaction between smooth muscle cells and the Fbg-L domain of tenascin-C is involved in cell adhesion and migration, and blocking this interaction would blunt SMC migration from media into the neointima and thereby affect neointimal formation, see LaFleur et al., J. Biol. Chem., 272(52):32798-32803, 1997. Further, cardiac myocyte activity involved tenascin, e.g., Yamamoto et al., J. Biol. Chem., (274) 31: 21840-21846, 1999.

Hyaluronan is also an extracellular matrix molecule that is useful for nanoparticles. Hyaluronan is preferentially expressed by hepatocytes and has been implicated angiogenesis. It is available in a variety of forms and has many known uses, e.g., as in U.S. Pat. No. 5,902,795.

Certain embodiments of coatings, components, and/or targets include natural and synthetic, native and modified, anionic or acidic saccharides, disaccharides, oligosaccharides, polysaccharides and glycosaminoglycans (GAGs). Dermatan sulfates, for example, have been shown to be useful for targeting molecules specifically to cells, e.g., as in U.S. Pat. No. 6,106,866.

Many peptidic fragments of extracellular matrix molecules are known that are bioactive functions, e.g, the tripeptidic integrin-mediated adhesion domain of fibronectin, see also, e.g., U.S. Pat. Nos. 6,074,659 and 5,646,248.

Moreover, other peptidic targeting ligands may be used, e.g., as in U.S. Pat. No. 5,846,561. Also, for example, lung targeting peptides are set forth in U.S. Pat. No. 6,174,867. Also, for example, organ targeting peptides may be used, as in U.S. Pat. No. 6,232,287. Also, for example, brain targeting peptides may be used, as in U.S. Pat. No. 6,296,832. Also, for example, heart-targeting peptides may be used, as in U.S. Pat. No. 6,303,5473.

Moreover, nanoparticles may be targeted for uptake by clatharin coated pits, as well as by caveolae, e.g., as in U.S. Pat. Nos. 5,284,646 and 5,554,386, which include carbohydrates for targeting uses.

Ligand-Conjugated Molecules

Certain embodiments are bioactive, diagnostic, or visualization agents that are conjugated to a cell recognition component or a cell recognition target. Such agents may be chemically attached to a cell recognition component, or other ligand, to target the therapeutic agents specifically to a cell or tissue. For example, a toxin may be conjugated to tenascin so as to deliver the toxin to a cancer cell. For example, a cell recognition component set forth herein may be conjugated to a bioactive, diagnostic, or visualization agent set forth herein. Conjugation may involve activating a bioactive, diagnostic, or visualization agent and/or the cell recognition component. Activating means to decorate with a chemical group that is capable of reacting with another chemical group to form a bond. Bonds may include, e.g., covalent and ionic bonds.

Embodiments include using a linking molecule having at least two functional groups that are activated and that react with the bioactive, diagnostic, or visualization agent and/or the cell recognition components so that they may be joined together. The bioactive, diagnostic, and/or visualization agents and/or the cell recognition component and/or the linking molecule may be activated.

The linking molecule may include a degradable group that is enzymatically or hydrolytically degradable so as to release the bioactive, diagnostic, or visualization agents. Examples of degradable groups include the polypeptide sequences cleaved by thrombin, plasmin, collagenase, intracellular proteases, and extracellular proteases. Other examples of degradable groups are lactides, caprolactones, and esters.

Chemistries for conjugating bioactive, diagnostic, or visualization agents to cell recognition components, e.g., proteins, peptides, antibodies, growth factors, ligands, and other cell recognition components or cell recognition targets are known to persons of ordinary skill in these arts, e.g., as in “Chemistry of Protein Conjugation and Cross-Linking” by Shan S. Wong, CRC Press; (Jun. 18, 1991) and Bioconjugate Techniques, Greg T. Hermanson, Academic Press, 1996, San Diego; and in U.S. Pat. No. 6,153,729 (especially as regards to polypeptides).

Moreover, the cell recognition component may be associated with delivery vehicles for delivering the therapeutic, diagnostic, or visualization agent. Examples of delivery vehicles include, e.g., liposomes, DNA particles, nanoparticles, stealth liposomes, polyethylene glycols, macromolecules, gels, hydrogels, controlled release matrices, sponges, degradable scaffolds, and microsponges.

Bioactive Agents

Embodiments include nanoparticles and particles that comprise bioactive agents that are delivered to cells and act to modulate cellular activity. To modulate cellular activity means to increase or decrease some aspect of cellular function, e.g., to increase or decrease synthesis of a protein or action of an enzyme. Bioactive agents or other agents may be delivered for many purposes. Agents can include drugs, proteins, small molecules, toxins, hormones, enzymes, nucleic acids, peptides, steroids, growth factors, modulators of enzyme activity, modulators of receptor activity and vitamins. By directing the agent towards the target where efficacy is to be obtained, and away from other areas where toxicity is obtained, particular cells and tissues can be targeted for research, scientific, and medical purposes. A tissue is a material made by the body, and may include extracellular matrix, structural proteins, and connective tissue. Tissues do not necessarily contain cells, but often do.

Growth factors are an example of a type of bioactive agent that may be delivered to a cell. As are discussed, growth factors are implicated in many cellular activities, particularly cell proliferation and differentiation. Thus growth factors may be used to modulate many cell activities, including hyperproliferation, differentiation, wound healing, bone formation, and other activities that are regulated by growth factors. Moreover, active moieties of growth factors e.g., polypeptides, are also known.

Small toxins are a type of agent that may be loaded into a nanoparticle and delivered to a cell or tissue. Many small toxins are known to those skilled in the metal parts, including toxins for use in treating cancer. Embodiments include nanoparticles loaded with small molecule toxins, including anthracyclines, doxorubicin, vincristine, cyclophosphamide, topotecan, taxol, and paclitaxel. These small toxins are, in general, predominantly hydrophobic and have relatively low MWs, about 1000 or less. Moreover, peptidic oncoagents are contemplated.

Further, compounds and agents that have been shown to be useful for modulating cellular activities for a therapeutic or diagnostic use are contemplated. For example, PCT WO 02/100343 describes the use of galectin for hyperproliferative disorders.

Apoptosis

Embodiments include nanoparticles and particles that comprise agents that modulate apoptosis, for example, by reducing or increasing the incidence of apoptosis. Apoptosis is a form of programmed cell death which occurs through the activation of cell-intrinsic suicide machinery. Apoptosis plays a major role during development and homeostasis. Apoptosis can be triggered in a variety of cell types by the deprivation of growth factors, which appear to repress an active suicide response. An apoptotic cell breaks apart into fragments of many apoptotic bodies that are rapidly phagocytosed. Inducing apoptosis in cancer cells can be an effective therapeutic approach. Inducing apoptosis in tissue cultured cells provides a model system for studying the effects of certain drugs for triggering, reversing, or halting the apoptotic pathway. Accordingly, increasing a cell's potential to enter the apoptotic pathway, or otherwise modulating apoptosis, is useful.

It is contemplated that the ability to inhibit apoptosis in a eukaryotic cell in tissue culture provides a model system for testing certain proteins and factors for their role in the apoptotic pathway. It also provides a model system for testing compounds suspected of being tumorigenic. In vitro such oligonucleotide containing nanoparticles may be administered by topical, injection, infusion or static coculture. In vivo administration of oligonucleotide containing nanoparticles can be subdermal, transdermal, subcutaneous, or intramuscular. Intravenous administration or use of implanted pumps may also be used. Doses are selected to provide effective inhibition of cancer cell growth and/or proliferation.

Specifically, some factors for modulating apoptosis include factors that activate or deactivate death receptors, including ligands for death receptors or factors that competitively inhibit the finding of factors to death receptors. Thus there are many factors that are modulators of apoptosis, i.e., that serve to enhance, inhibit, trigger, initiate, or otherwise affect apoptosis. Apoptosis may be triggered by administration of apoptotic factors, including synthetic and natural factors. Some natural factors interact with cell surface receptors referred to death receptors and contribute to, or cause, apoptosis. Death receptors belong to the tumor necrosis factor (TNF) gene superfamily and generally can have several functions other than initiating apoptosis. The best characterized of the death receptors are CD95 (or Fas), TNFR1 (TNF receptor-1) and the TRAIL (TNF-related apoptosis inducing ligand) receptors DR4 and DR5.

The bcl-2 proteins are a family of proteins involved in the response to apoptosis. Some of these proteins (such as bcl-2 and bcl-XL) are anti-apoptotic, while others (such as Bad or Bax) are pro-apoptotic. The sensitivity of cells to apoptotic stimuli can depend on the balance of pro- and anti-apoptotic bcl-2 proteins. Thus some factors for modulating apoptosis or factors that up regulate or down regulate bcl-2 proteins, modulate bcl-2 proteins, competitively inhibit such proteins, specifically behind such proteins, or active fragments thereof. Moreover, delivery of bcl-2 proteins can modulate apoptosis.

Caspases are a family of proteins that are effectors of apoptosis. The caspases exist within the cell as inactive pro-forms or zymogens. The zymogens can be cleaved to form active enzymes following the induction of apoptosis. Induction of apoptosis via death receptors results in the activation of an initiator caspase. These caspases can then activate other caspases in a cascade that leads to degradation of key cellular proteins and apoptosis. Thus some factors for modulating apoptosis are factors that up regulate or down regulate caspases, modulate caspases, competitively inhibit caspases, specifically behind caspases, or active fragments thereof. Moreover, delivery of caspases can modulate apoptosis. About 13 caspases are presently known, and are referred to as caspase-1, caspases-2, etc.

Aside from the ligation of death receptors, there are other mechanisms by which the caspase cascade can be activated. For example, Granzyme B can be delivered into cells and thereby directly activate certain caspases. For example, delivery of cytochrome C can also lead to the activation of certain caspases.

An example of an apoptosis modulating factor is CK2α. CK2α potentiates apoptosis in a eukaryotic cell. CK2 biological activity may be reduced by administering to the cell an effective amount of an anti-sense stand of DNA, RNA, or siRNA. An embodiment is the use of nanoparticles to potentiate apoptosis in eukaryotic cells by decreasing the expression of casein-kinase-2. Apoptosis is inhibited or substantially decreased by preventing transcription of CK-2 DNA and/or translation of RNA. This can be carried out by introducing antisense oligonucleotides of the CK-2 sequence into cells, in which they hybridize to the CK-2 encoding mRNA sequences, preventing their further processing. It is contemplated that the antisense oligonucleotide can be introduced into the cells by introducing antisense-single stranded nucleic acid which is substantially identical to the complement of the cDNA sequence. It is also possible to inhibit expression of CK-2 by the addition of agents which degrade CK-2. Such agents include a protease or other substance which enhances CK-2 breakdown in cells. In either case, the effect is indirect, in that less CK-2 is available than would otherwise be the case.

Nucleic Acids

As used herein, the term nucleic acid refers to both RNA and DNA, including cDNA, genomic DNA, synthetic (e.g., chemically synthesized) DNA, as well as naturally-occurring and chemically modified nucleic acids, e.g., synthetic bases or alternative backbones. A nucleic acid molecule can be double-stranded or single-stranded (i.e., a sense or an antisense single strand).

Polynucleic acids, such as the sequences set forth herein and fragments thereof, can be used in diagnostics, therapeutics, prophylaxis, and as research reagents and in kits. Provision of means for detecting hybridization of oligonucleotide with a gene, mRNA, or polypeptide can routinely be accomplished. Such provision may include enzyme conjugation, radiolabeling or any other suitable detection systems. Research purposes are also available, e.g., specific hybridization exhibited by the polynucleotides or polynucleic acids may be used for assays, purifications, cellular product preparations and in other methodologies which may be appreciated by persons of ordinary skill in the art.

Polynucleotides are nucleic acid molecules of at least three nucleotide subunits. A nucleotide, as the term is used herein, has three components: an organic base (e.g., adenine, cytosine, guanine, thymine, or uracil, herein referred to as A, C, G, T, and U, respectively), a phosphate group, and a five-carbon sugar that links the phosphate group and the organic base. In a polynucleotide, the organic bases of the nucleotide subunits determine the sequence of the polynucleotide and allow for interaction with a second polynucleotide. The nucleotide subunits of a polynucleotide are linked by phosphodiester bonds such that the five-carbon sugar of one nucleotide forms an ester bond with the phosphate of an adjacent nucleotide, and the resulting sugar-phosphates form the backbone of the polynucleotide. Polynucleotides described herein can be produced through the well-known and routinely used technique of solid phase synthesis. Similarly, a polynucleotide has a sequence of at least three nucleic acids and may be synthesized using commonly known techniques.

Polynucleotides and polynucleotide analogues (e.g., morpholinos) can be designed to hybridize to a target nucleic acid molecule. The term hybridization, as used herein, means hydrogen bonding, which can be Watson-Crick, Hoogsteen, or reversed Hoogsteen hydrogen bonding, between complementary nucleoside or nucleotide bases. For example, A and T, and G and C, respectively, are complementary bases that pair through the formation of hydrogen bonds. Complementary, as used herein, refers to the capacity for precise pairing between two nucleotides. A nonspecific adsorption or interaction is not considered to be hybridization. For example, if a nucleotide at a certain position of a polynucleotide analogue is capable of hydrogen bonding with a nucleotide at the same position of a target nucleic acid molecule, then the polynucleotide analogue and the target nucleic acid molecule are considered to be complementary to each other at that position. A polynucleotide or polynucleotide analogue and a target nucleic acid molecule are complementary to each other when a sufficient number of corresponding positions in each molecule are occupied by nucleotides that can hydrogen bond with each other. It is understood in the art that the sequence of the polynucleotide or polynucleotide analogue need not be 100% complementary to that of the target nucleic acid molecule to hybridize.

Certain embodiments provide various polypeptide sequences and/or purified polypeptides. A polypeptide refers to a chain of amino acid residues, regardless of post-translational modification (e.g., phosphorylation or glycosylation) and/or complexation with additional polypeptides, synthesis into multisubunit complexes, with nucleic acids and/or carbohydrates, or other molecules. Proteoglycans therefore also are referred to herein as polypeptides. A functional polypeptide is a polypeptide that is capable of promoting the indicated function. Polypeptides can be produced by a number of methods, many of which are well known in the art.

The term purified as used herein with reference to a polypeptide refers to a polypeptide that either has no naturally occurring counterpart (e.g., a peptidomimetic), or has been chemically synthesized and is thus substantially uncontaminated by other polypeptides, or has been separated or purified from other most cellular components by which it is naturally accompanied (e.g., other cellular proteins, polynucleotides, or cellular components). An example of a purified polypeptide is one that is at least 70%, by dry weight, free from the proteins and naturally occurring organic molecules with which it naturally associates. A preparation of the a purified polypeptide therefore can be, for example, at least 80%, at least 90%, or at least 99%, by dry weight, the polypeptide. Polypeptides also can be engineered to contain a tag sequence (e.g., a polyhistidine tag, a myc tag) that facilitates the polypeptide to be purified or marked (e.g., captured onto an affinity matrix, visualized under a microscope).

Vectors

Nucleic acids can be incorporated into vectors. As used herein, a vector is a replicon, such as a plasmid, phage, or cosmid, into which another nucleic acid segment may be inserted so as to bring about replication of the inserted segment. Vectors of the invention typically are expression vectors containing an inserted nucleic acid segment that is operably linked to expression control sequences. An expression vector is a vector that includes one or more expression control sequences, and an expression control sequence is a DNA sequence that controls and regulates the transcription and/or translation of another DNA sequence. Expression control sequences include, for example, promoter sequences, transcriptional enhancer elements, and any other nucleic acid elements required for RNA polymerase binding, initiation, or termination of transcription. With respect to expression control sequences, “operably linked” means that the expression control sequence and the inserted nucleic acid sequence of interest are positioned such that the inserted sequence is transcribed (e.g., when the vector is introduced into a host cell). For example, a DNA sequence is operably linked to an expression-control sequence, such as a promoter when the expression control sequence controls and regulates the transcription and translation of that DNA sequence. The term “operably linked” includes having an appropriate start signal (e.g., ATG) in front of the DNA sequence to be expressed and maintaining the correct reading frame to permit expression of the DNA sequence under the control of the expression control sequence to yield production of the desired protein product. Examples of vectors include, for example, plasmids, adenovirus, Adeno-Associated Virus (AAV), Lentivirus (FIV), Retrovirus (MoMLV), and transposons, e.g., as set forth in U.S. Pat. No. 6,489,458.

There are a variety of promoters that could be used including, e.g., constitutive promoters, tissue-specific promoters, inducible promoters, and the like. Promoters are regulatory signals that bind RNA polymerase in a cell to initiate transcription of a downstream (3′ direction) coding sequence.

Antisense

Anti-sense DNA compounds (e.g., oligonucleotides) treat disease, and more generally later biological activity, by interrupting cellular production of a target protein. Such compounds offer the potential benefits of 1) rational drug design rather than screening huge compound libraries and 2) a decrease in anticipated side effects due to the specificity of Watson-Crick base-pairing between the antisense molecule's sequential pattern of nucleotide bases and that of the target protein's precursor mRNA. One antisense therapeutic, Vitravene, has been approved for human use in the treatment of AIDS-related CMV retinitis. This drug is applied by intravitreol injection, which aids in maintaining drug concentration due to the isolation of the eye compartment from the systemic circulation.

A polynucleic acid or polynucleic acid analogue can be complementary to a sense or an antisense target nucleic acid molecule. When complementary to a sense nucleic acid molecule, the polynucleic acid is said to be antisense. Thus the identification as sense or antisense is referenced to a particular reference nucleic acid. For example, a polynucleotide analogue can be antisense to an mRNA molecule or sense to the DNA molecule from which an mRNA is transcribed. As used herein, the term “coding region” refers to the portion of a nucleic acid molecule encoding an RNA molecule that is translated into protein. A polynucleotide or polynucleotide analogue can be complementary to the coding region of an mRNA molecule or the region corresponding to the coding region on the antisense DNA strand. Alternatively, a polynucleotide or polynucleotide analogue can be complementary to the non-coding region of a nucleic acid molecule. A non-coding region can be, for example, upstream of a transcriptional start site or downstream of a transcriptional end-point in a DNA molecule. A non-coding region also can be upstream of the translational start codon or downstream of the stop codon in an mRNA molecule. Furthermore, a polynucleotide or polynucleotide analogue can be complementary to both coding and non-coding regions of a target nucleic acid molecule. For example, a polynucleotide analogue can be complementary to a region that includes a portion of the 5′ untranslated region (5′-UTR) leading up to the start codon, the start codon, and coding sequences immediately following the start codon of a target nucleic acid molecule.

Various antisense molecules are set forth herein. In some embodiments, the antisense molecules can be preferably targeted to hybridize to the start codon of a mRNA and to codons on either side of the start codon, e.g., within 1-20 bases of the start codon. Other codons, however, may be targeted with success, e.g., any set of codons in a sequence. The procedure for identifying additional antisense molecules will be apparent to an artisan of ordinary skill after reading this disclosure. One procedure would be to test antisense molecules of about 20 nucleic acids in a screening assay. Each proposed antisense molecule would be tested to determine its effectiveness, and the most promising candidates would form the basis for optimization.

Hybridization of antisense oligonucleotides with mRNA interferes with one or more of the normal functions of mRNA, e.g., translocation of the RNA to a site of protein translation, translation of protein from the RNA, splicing of the RNA to yield one or more mRNA species, and catalytic activity which may be engaged in by the RNA. Binding of specific protein(s) to the RNA may also be interfered with by antisense oligonucleotide hybridization to the RNA.

The function of a gene can be disrupted by delivery of anti-sense DNA or RNA that prevents transcription or translation of the protein encoded by the gene. This can be accomplished by providing an appropriate length oligonucleotide which is complimentary to at least a portion of the messenger RNA (mRNA) transcribed from the gene. The antisense strand hybridizes with the mRNA and targets mRNA destruction by preventing ribosomal translation, and subsequent protein synthesis. The specificity of antisense oligonucleotides arises from the formation of Watson-Crick base pairing between the heterocyclic bases of the oligonucleotide and complimentary bases on the target nucleic acid. Oligonucleotides of greater length (15-30 bases) are preferred because they are more specific, and are less likely to induce toxic complications that might result from unwanted hybridization.

The incorporation of small interfering RNA (SiRNA) molecules, which are double stranded RNA molecules that are capable of mimicking an RNA virus infection. One advantage of using SiRNA molecules is that such molecules are very easy to design. In fact, SiRNA molecules may be based on any portion of a messenger RNA molecule or transcript and still be effective in delivering a therapeutic effect in a target cell. As an example, the casein kinase 2 mRNA transcript may be used to prepare an SiRNA molecule. Furthermore, SiRNA molecules typically have little, if any, binding issues since the SiRNA molecule need not bind to specific portion of the gene in order to be effective.

CK2α Antisense

An example of a system for delivering antisense molecules is a collection of nanoparticles of less than about 200 nm loaded with CK2α and optionally made with tenascin or other cell-specific targeting molecules. Other antisense molecules, including those directed against subunits of CK2α, may alternatively be used.

Shown herein, see Examples, are nanoparticles loaded with antisense CK2 used to treat a chemoresistant head neck carcinoma line (SCC-15) in vitro and in vivo. Using a phosphodiester DNA oligomer targeted to the translation initiation site, the Applicant has shown an increase in efficacy in vitro for this embodiment as compared to liposomal antisense CK2 and cisplatin (Unger, 2002). The Applicant has also shown a dose response against 1 mm tumor nests cultured in vitro and have shown biological activity against pilot 4 mm xenograft tumors grown in nude mice (Unger, 2002). See also Examples.

CK2, historically known as Casein Kinase 2, is a constitutively active kinase with over 160 subtargets throughout the cell including proteins critical in ribosome synthesis, nucleic acid synthesis and repair, nuclear and cytoplasmic cytoskeletal rearrangement, transcription of both oncogenes and tumor suppressor genes, mitochondrial function and cell cycle control (reviewed in Faust et al., 2000). In primary human tumors tested to date (8 types), CK2 is upregulated 2 to 8 fold by kinase activity of crude homogenates or nuclear-localized protein levels suggesting a role in cell viability.

Not surprisingly, CK2 exhibits complex spatial-temporal localization patterns consistent with its concurrent regulatory activity over multiple cellular processes. In in vitro studies conducted with prostate carcinoma lines, CK2 translocation from the cytosol to the nuclear matrix precedes proliferation activity, while following application of cytotoxic drugs, translocation to the cytosol precedes induction of apoptosis. Several lines of investigation support the notion that shuttling of CK2 to the nucleus (e.g. nuclear matrix and chromatin) is related to regulation of cell growth and apoptosis suppression. Rapid loss of CK2 from the nucleus is associated with cessation of cell growth, an indication of apoptosis.

Prostate and SCCHN carcinoma cells appear vulnerable to antisense manipulation of CK2 protein levels. A 2 μg/ml liposomal dose of a phosphorothioate 20 mer directed to the translation initiation site of CK2, induced a 55% apoptosis incidence concomitant with a 36% reduction in specific nuclear CK2 activity and a 42% decrease in nuclear protein levels. A 20% decrease in protein with no reduction in activity was induced by a nonsense control. These studies showed that even a modest reduction of CK2 in the nucleus resulted in extensive apoptosis.

In head neck tumor biopsies, CK2 is upregulated and increased levels negatively correlate with tumor grade, stage and clinical outcome. Immunohistochemical analysis of prostate and SCCHN tumors reveals that CK2 is additionally upregulated in the nuclear compartment of cells in the periphery of tumor. This may relate to the consideration that the advancing edge of a solid tumor has the capacity to secrete soluble factors that can facilitate invasion of local stroma. These studies point to the involvement of CK2 in multiple aspects of tumor biology including differentiation, invasion, metastasis and response to therapy.

As shown in the Examples herein, or previously, nanoparticles of less than about 50 n made with hydrophilic surfactants and the extracellular matrix protein tenascin selectively deliver nucleic acid cargo to solid tumors. This selective uptake is mediated by caveolar endocytosis. Nanoparticle entry into solid tumors is from the surrounding tissue (peritumoral infiltration). Local delivery via peritumoral infiltration may offer advantages over current delivery methods into solid tumors. Further increases in drug efficacy are expected to be obtained by incorporating formats exhibiting higher binding affinities for the target Protein Kinase CK2 mRNA.

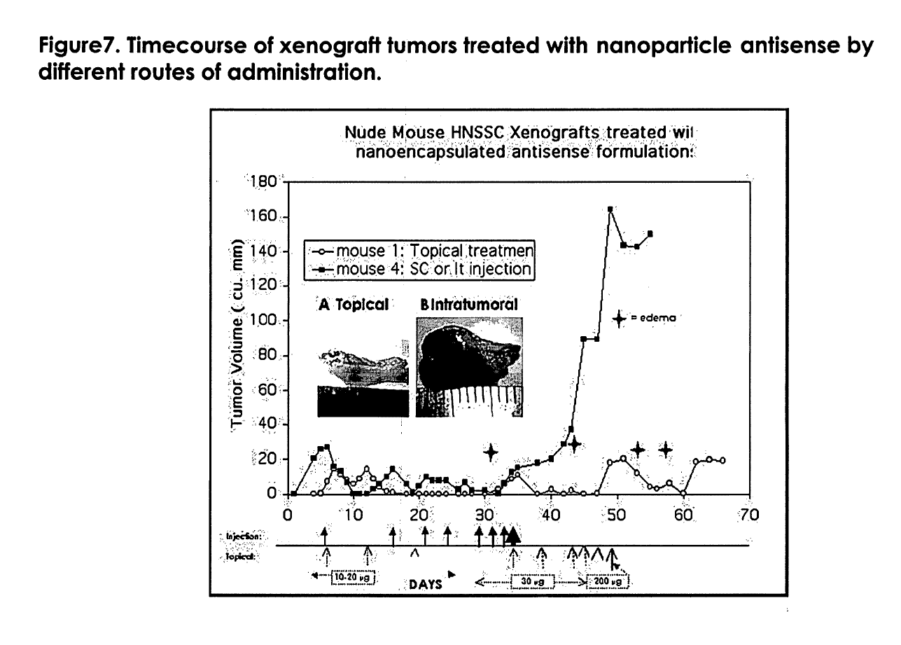

The effectiveness of CK2α nanoparticles was further confirmed using live mouse models. One mouse was treated topically and the other by injection. Nude mice were injected dorsally with 2(10)⁶ SSC-15 cells and treatment began when tumors were palpable (3×4 mm). FIG. 7 shows that topical treatment was more effective than injection. Mice were initially treated mice with single small doses (10-30 μg) and it was found that tumors would regress completely but eventually return. With repeat dosing as time went on, the interval between reappearance decreased suggested that less than complete kill selected for more aggressive cells. Finally, mice were treated with a single 200 μg dose of a collection of nanoparticles of less than about 50 nm diameter loaded with CK2α antisense, either topically or by intratumoral injection and then followed without further treatment for an additional 2 week. This dose was chosen as being below the typical dose (20 mg/kg) that hematological toxicities appear in mice treated with nuclease-resistant phosphorothioates with repeat i.v. administration. Both tumors were 3×4 mm at time of treatment. After 2 weeks, tumor volume had increased 8-fold in the mouse treated by injection while the topically-treated tumor regressed to become transiently inflamed and edematous. Next we examined center sections from the excised tumors to determine the incidence of apoptosis and fate of the carcinoma cells in the topical tumor. Using fluorescence microscopy we detected for activated Caspase 3, and found that it was present, indicating that the antisense caused apoptosis.

Antisense Chemistries

Polynucleotide analogues or polynucleic acids can be chemically modified polynucleotides or polynucleic acids. In some embodiments, polynucleotide analogues can be generated by replacing portions of the sugar-phosphate backbone of a polynucleotide with alternative functional groups. these chemical modifications can be combined within one antisense structure. For example, a portion of the polynucleic acid can comprise an unmodified phosphodiester DNA or RNA (DNA/RNA) linkage between two nucleotides combined with one or more DNA/RNA modifications at other positions in the molecule. The modifications can be at internucleoside linkages or at the ends of the polynucleic acid.

Morpholino-modified polynucleotides, referred to herein as “morpholinos,” are polynucleotide analogues in which the bases are linked by a morpholino-phosphorodiamidate backbone (See, Summerton and Weller (1997) Antisense Nuc. Acid Drug Devel. 7:187-195; and U.S. Pat. Nos. 5,142,047 and 5,185,444). In addition to morpholinos, other examples of polynucleotide analogues include analogues in which the bases are linked by a polyvinyl backbone (Pitha et al. (1970) Biochim. Biophys. Acta 204:39-48; Pitha et al. (1970) Biopolymers 9:965-977), peptide nucleic acids (PNAs) in which the bases are linked by amide bonds formed by pseudopeptide 2-aminoethyl-glycine groups (Nielsen et al. (1991) Science 254:1497-1500), analogues in which the nucleoside subunits are linked by methylphosphonate groups (Miller et al. (1979) Biochem. 18:5134-5143; Miller et al. (1980) J. Biol. Chem. 255:9659-9665), analogues in which the phosphate residues linking nucleoside subunits are replaced by phosphoroamidate groups (Froehler et al. (1988) Nucleic Acids Res. 156:4831-4839), and phosphorothioated DNAs, analogues containing sugar moieties that have 2′ O-methyl groups (Cook (1998) Antisense Medicinal Chemistry, Springer, N.Y., pp. 51-101), such as p-ethoxy-2′omethyl RNA modifications. Modifications can be performed at the terminal sugars of the polynucleic acid, such as 2′ modified RNA, 3′ end-blocked RNA and 5′ end blocked RNA.

Polynucleic acids and polynucleic acid analogue embodiments can be useful for research and diagnostics, and for therapeutic use. Modified nucleic acids are known and may be used with embodiments described herein, for example as described in Antisense Research and Application (Springer-Verlag, Berlin, 1998), and especially as described in the chapter by S. T. Crooke: Chapter 1: Basic Principles of Antisense Therapeutics pp. 1-50; and in Chapter 2 by P. D. Cook: Antisense Medicinal Chemistry pp. 51-101. Some modified backbones for nucleic acid molecules are, for example, morpholinos, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Much progress has been made in optimizing the backbone structure of oligonucleotides to optimize the following features; 1) increased stability in the presence of destructive blood-borne nucleases, 2) high affinity binding with the mRNA target, 3) increased water solubility and/or 4) increased specificity by utilization of non-RNAse H mechanisms. Systems that are being used for in vitro antisense studies include mechanical means (microinjection, particle bombardment), electrical means (electroporation), chemical/intracellular delivery (lipids, cationic polymers, nanoparticles and proteins) and chemical/permeabilization (streptolysin 0, amphotericin B). All of these systems, however, are directed to cellular uptake routes that expose the delivered agent to lysosomal sequestration and destruction by the endosomal pathway.

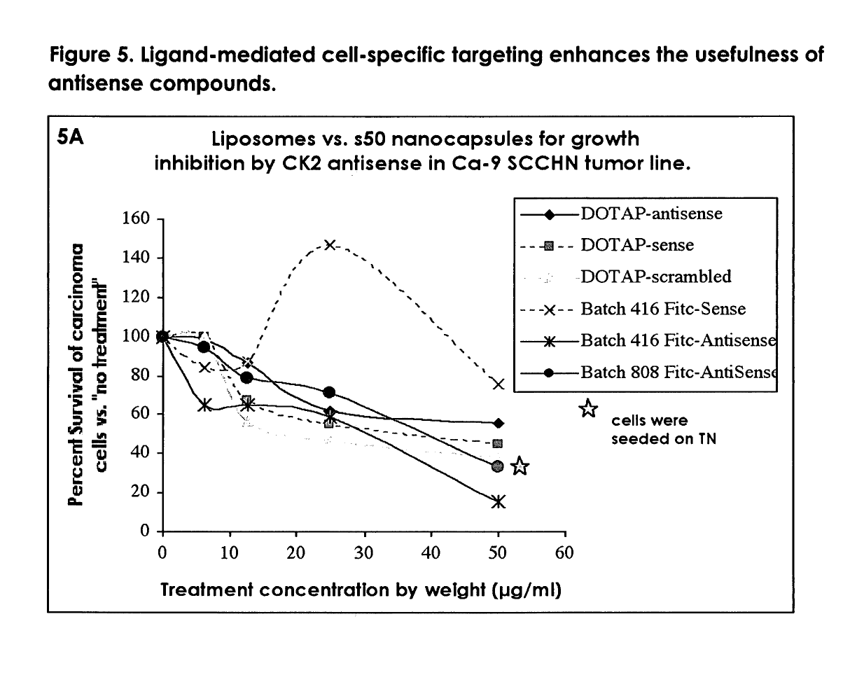

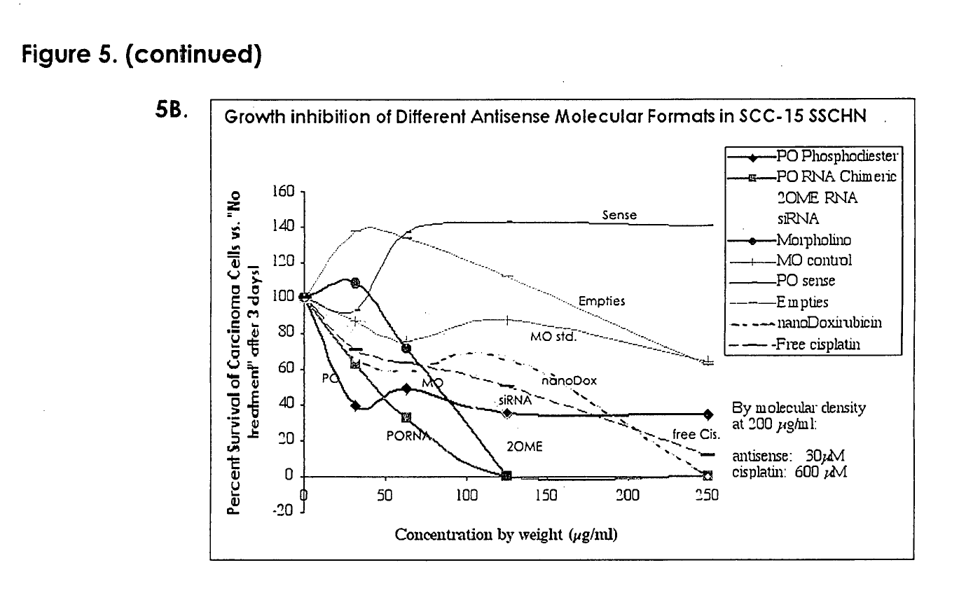

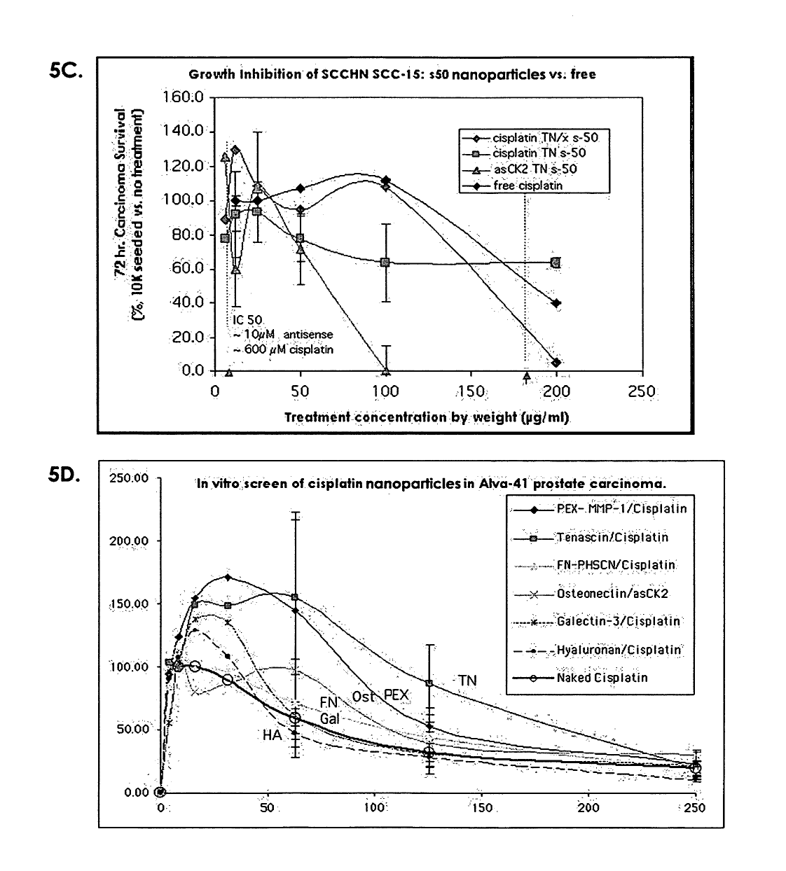

The efficacies of various nucleic acid backbone chemistries were investigated by delivering cisplatin to cancer cells in organ culture using a collection of nanoparticles that were less than about 50 nm in diameter. Recurrent head neck tumors are typically small (1-2 cm), but based on volumetric scaling between in vitro tumor nests and mouse studies, it is estimated that estimate that a dose of 3 5 mg will be required to locally treat a 2 cm tumor. Various nucleic acid chemistries may reduce this amount by either enhancing binding affinity between the target mRNA and the antisense, using the antisense to bind to DNA instead of RNA, or increasing nuclease resistance (and half-life). FIG. 5 shows the results of testing the various antisense backbones. Biological activity was assayed as growth inhibition using the MTT/WST assay in a 96 well format. Cells were seeded at 20,000 per well, treated 18 hours later, then assayed at 72 hours post treatment. Although the cells are resistant to conventional chemotherapeutic agents, cisplatin activity is shown for reference (black line). The results indicate that phosphodiester Asnan has an IC, of 30[tg/ml (5˜tM), but is only partially effective in vitro. A complete kill of only 60% is achieved suggesting potentially issues with early intracellular degradation (dashed line). Alternatively, the 2-0 methyl RNA format shows an IC, of approximately 150 pg/ml (20 [M) with the capacity for complete kill in vitro (purple line). Additional formats screened but not shown were a phosphodiester/2OME chimeric and the siRNA format. Performance was similar to the 2OME with lower efficacy.

Antibodies

Nanoparticles can comprise antibodies for targeting the nanoparticles to cells or tissues, whereby bioactive or visualization agents associated with the nanoparticles may be delivered. Some embodiments include antibodies having specific binding activity for a cell recognition target, e.g., cell surface receptor, extracellular matrix molecule, growth factor receptor, or cell specific marker. Such antibodies can be useful for directing nanoparticles to specific cell types, for example. The term antibody or antibodies includes intact molecules as well as fragments thereof that are capable of binding to an epitope. The term “epitope” refers to an antigenic determinant on an antigen to which an antibody binds. The terms antibody and antibodies include polyclonal antibodies, monoclonal antibodies, humanized or chimeric antibodies, single chain Fv antibody fragments, Fab fragments, and F(ab)₂ fragments.

Antibodies may be generated according to methods known to those skilled in these arts, e.g., recombinantly, or via hybridoma processes. Further, monoclonal antibodies can be obtained by any technique that provides for the production of antibody molecules by, for example, continuous cell lines in culture as described by Kohler et al. (1975) Nature 256:495-497; the human B-cell hybridoma technique of Kosbor et al. (1983) Immunology Today 4:72 and Cote et al. (1983) Proc. Natl. Acad. Sci. USA 80:2026-2030; and the EBV-hybridoma technique of Cole et al. Monoclonal Antibodies and Cancer Therapy, Alan R. Liss, Inc. pp. 77-96 (1983). Such antibodies can be of any immunoglobulin class, including IgM, IgG, IgE, IgA, IgD, and any subclass thereof. A hybridoma producing the monoclonal antibodies of the invention can be cultivated in vitro or in vivo. A chimeric antibody can be a molecule in which different portions are derived from different animal species, such as those having a variable region derived from a mouse monoclonal antibody and a human immunoglobulin constant region. Chimeric antibodies can be produced through standard techniques.

A monoclonal antibody also can be obtained by using commercially available kits that aid in preparing and screening antibody phage display libraries. An antibody phage display library is a library of recombinant combinatorial immunoglobulin molecules. Examples of kits that can be used to prepare and screen antibody phage display libraries include the Recombinant Phage Antibody System (Pharmacia, Peapack, N.J.) and SurfZAP Phage Display Kit (Stratagene, La Jolla, Calif.). Once produced, antibodies or fragments thereof can be tested for recognition of a polypeptide by standard immunoassay methods including, for example, enzyme-linked immunosorbent assay (ELISA) or radioimmuno assay (RIA).

Cell Specific Targeting

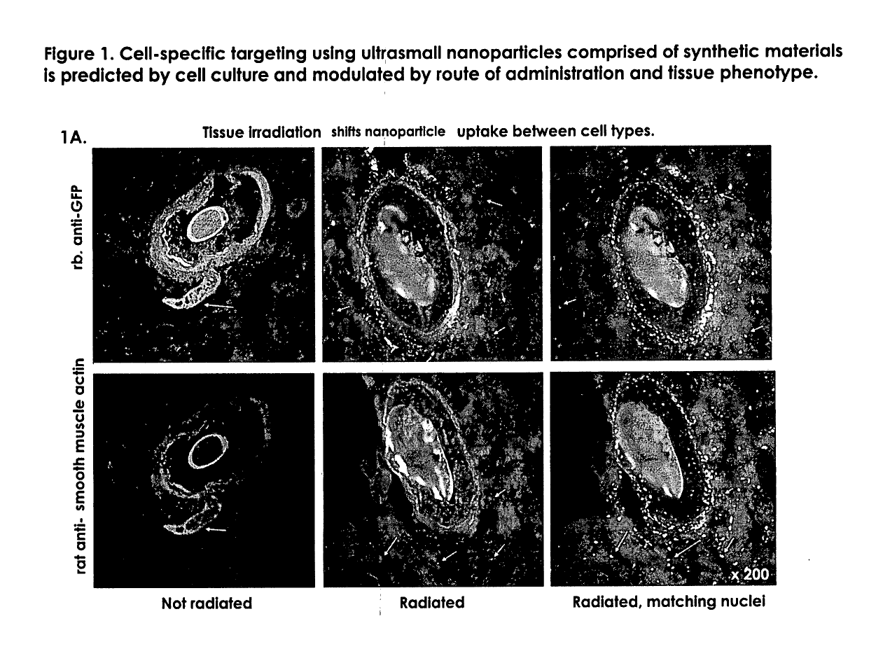

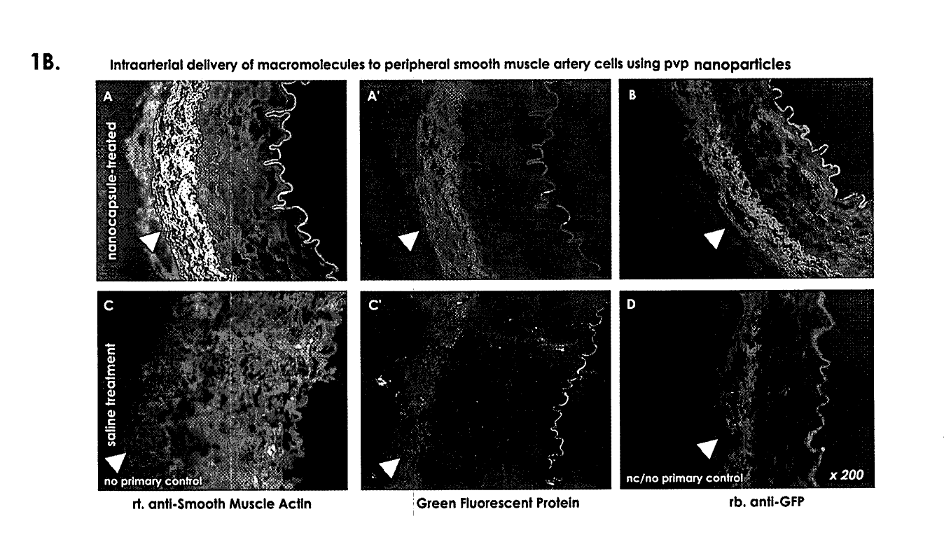

One method of targeting a cell or tissue is to deliver nanoparticles, e.g., nanocapsules, directly to a location at or near the cell or tissue, e.g., by use of a needle, catheter, transcutaneous delivery system, or suppository. Example 1 shows how s50 nanoparticles made with polymeric component are taken up by cells in the vicinity of the site of administration. In Example 1, pvp nanoparticles were delivered to organ cultures and were observed to be taken up by both smooth muscle cells and fibroblasts. When cell phenotypes were shifted to myofibroblasts, however, the myofibroblasts preferentially took up the pvp nanoparticles (FIGS. 1A and 1B). Radiation fibrosis and scarring diseases are characterized by abnormal proliferation and/or activity myofibroblasts. Therefore these conditions may be treated by introducing nanoparticles comprising bioactive agents to regions wherein myofibroblasts are present so that the cells will take up the nanoparticles and receive the bioactive agents, which could be chosen to modulate the activity of myofibroblasts. Examples of bioactive agents that modulate myofibroblasts include, e.g., toxins, cell proliferation inhibitors, DNA synthesis inhibitors, DNA replication inhibitors, apoptosis agents, and antisense molecules that inhibit DNA transcription.

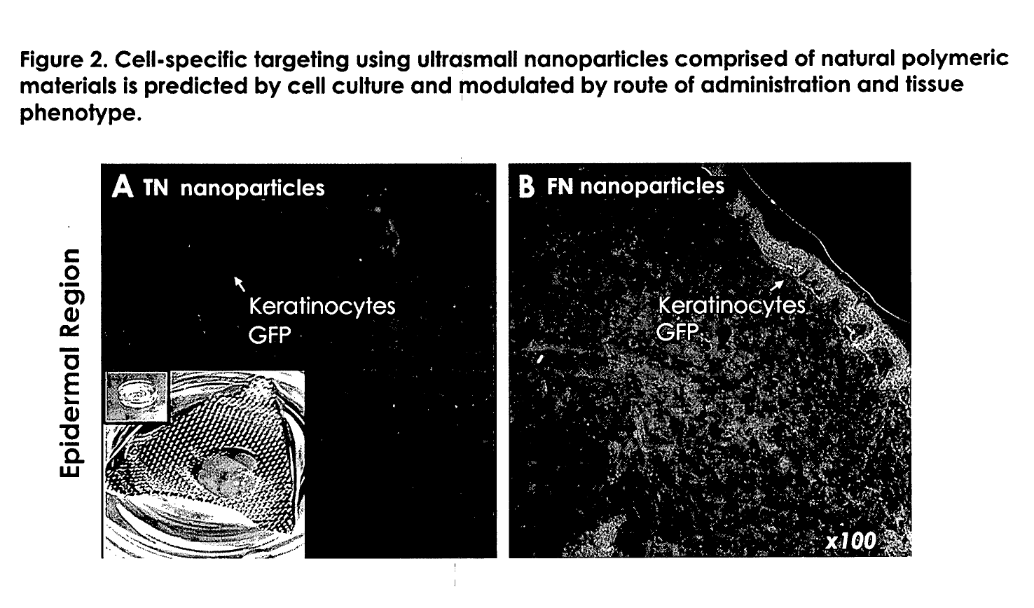

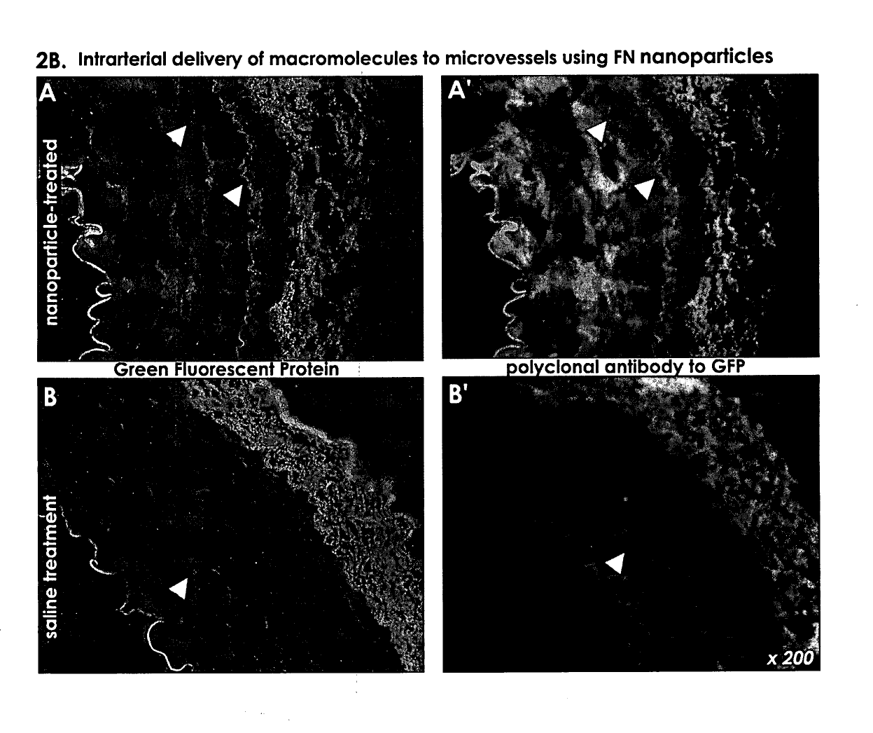

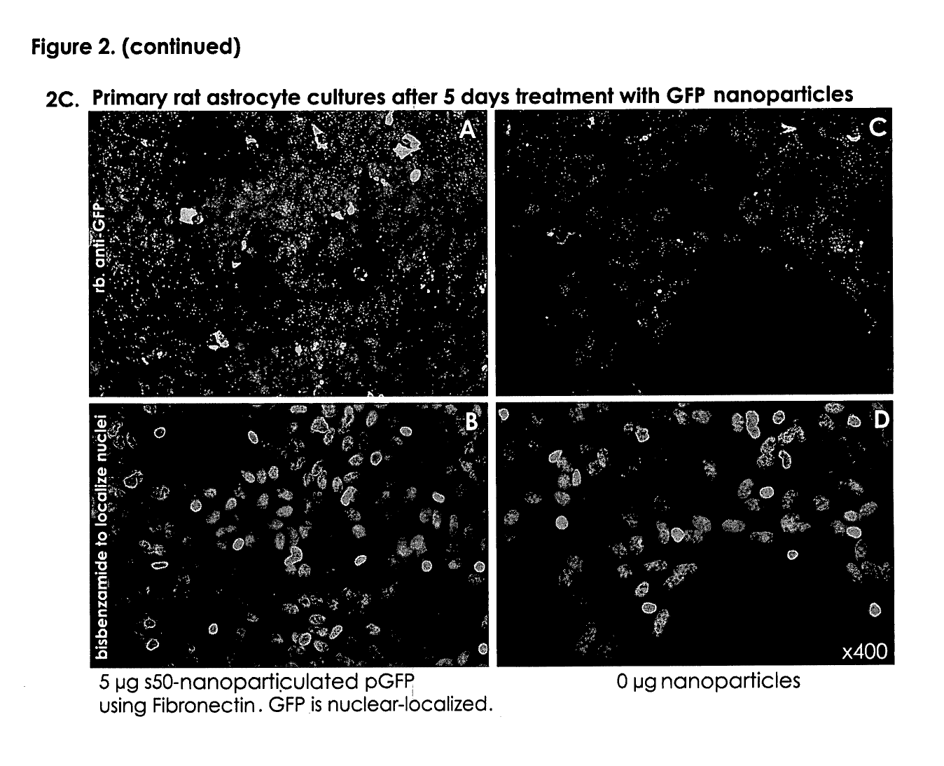

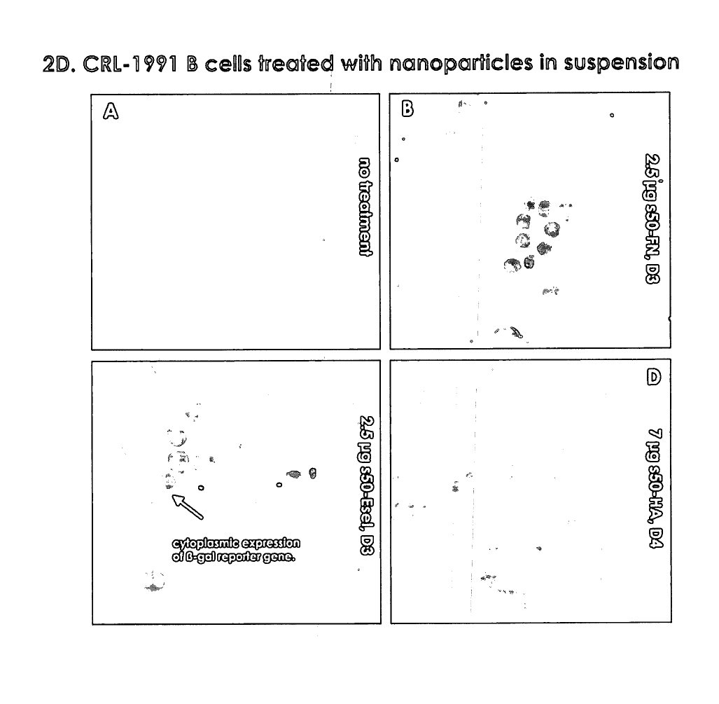

Nanoparticles penetrate tissues and are able to reach cells for which they are targeted. Thus s50 nanoparticles comprising ligands that are targeted to certain cell types will preferentially interact with the targeted cells instead of other cells. This behavior is shown in Example 1, and FIGS. 1A, 1B, and 1C. Nanoparticles made of pvp were preferential for smooth muscle cells and fibroblasts (FIG. 1A) and, when injected into a blood vessel lumen, penetrated the intima, penetrated the media, and penetrated the adventitia, where they were taken up by actin-positive cells, e.g., smooth muscle cells. These nanoparticles thus bypassed other cells, including a monolayer of endothelial cells, to reach the target tissue. These experiments also show that nanoparticles may also be used to specifically target cells or tissues in the adventitia of a blood vessel, e.g., an artery. Thus nanoparticles having bioactive agents may be delivered to a blood vessel adventitia by delivering them to the lumen of the blood vessel. Cells in or near the adventitia take up the nanoparticles and are thereby affected by the bioactive agent. Further, medial cells of the vasculature could be targeted using fibronectin s50 nanoparticles, without affecting cells of the adventitia or intima (FIG. 2B). Numerous ligands specific for endothelial cells are set forth herein and are known to those of ordinary skill in these arts so that endothelial cells may also be targeted, as well as other cells of the vasculature. It is possible to target cells of the vasculature using nanoparticles, e.g., s50 nanoparticles, and to deliver bioactive agents, as well as other agents that may be associate with the nanoparticles, to the cells. Some specific cell types suitable for targeting include, for example, glial cells, astrocytes, smooth muscle cells, myofibroblasts, vascular endothelial cells, leukaemic blasts, vascular endothelial cells in solid tumors, B-cell lymphoproliferative disease cells, acute myeloid leukemia cells, glial tumor cells, breast cancer cells, small-cell lung cancer cells, ovarian cancer cells, colorectal cancer cells, blood vessel medial cells, squamous cell carcinoma cells and epithelial-derived cancer cells.

Topical administration to epidermis of s50 nanoparticles made with fibronectin, FIG. 2A, showed that keratinocytes could be specifically targeted. Other studies showed that astrocytes and neurons took up fibronectin s50 nanoparticles with great efficiency (FIGS. 2C and 2D). And other results showed that hyaluronan s50 nanoparticles were taken up by B cells (FIG. 2D).

Other results confirm that nanoparticles may be targeted to a cell and be expected to interact specifically with that cell. When nanoparticles comprising tenascin were targeted to cells that preferentially express the tenascin receptor, the uptake of the nanoparticles was inhibited by the presence of free tenascin. This result shows that the tenascin s50 nanoparticles interacted with the cells using a mechanism that specifically involved tenascin. Thus other cells can be targeted using s50 nanoparticles that have factors that are specific for targets on those cells and can be expected to be preferentially taken up by those cells.

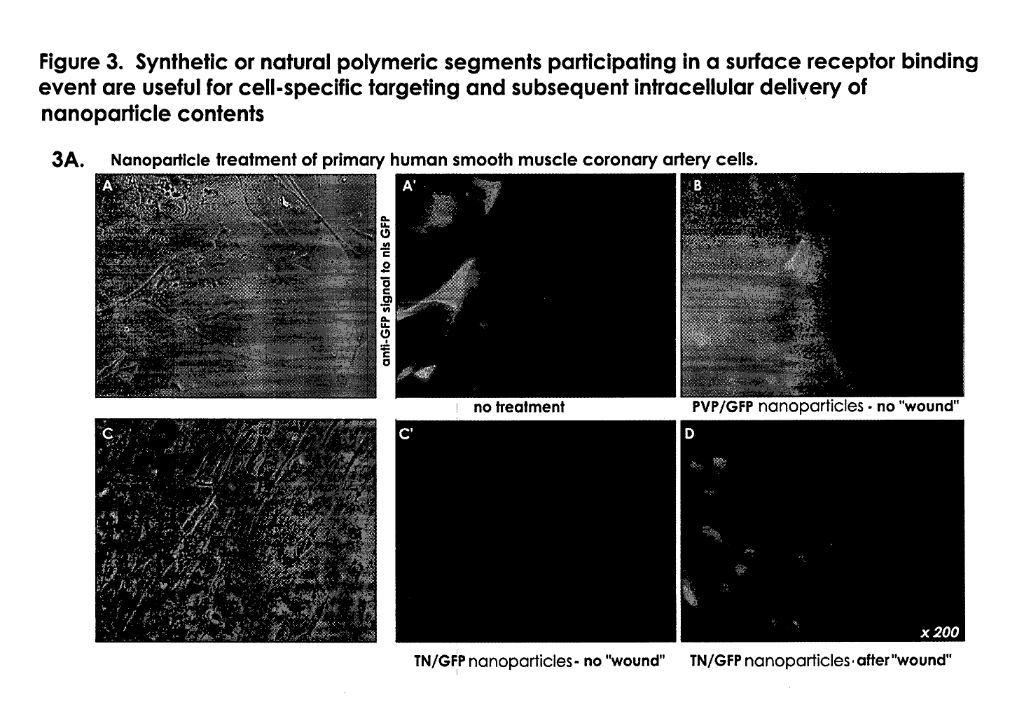

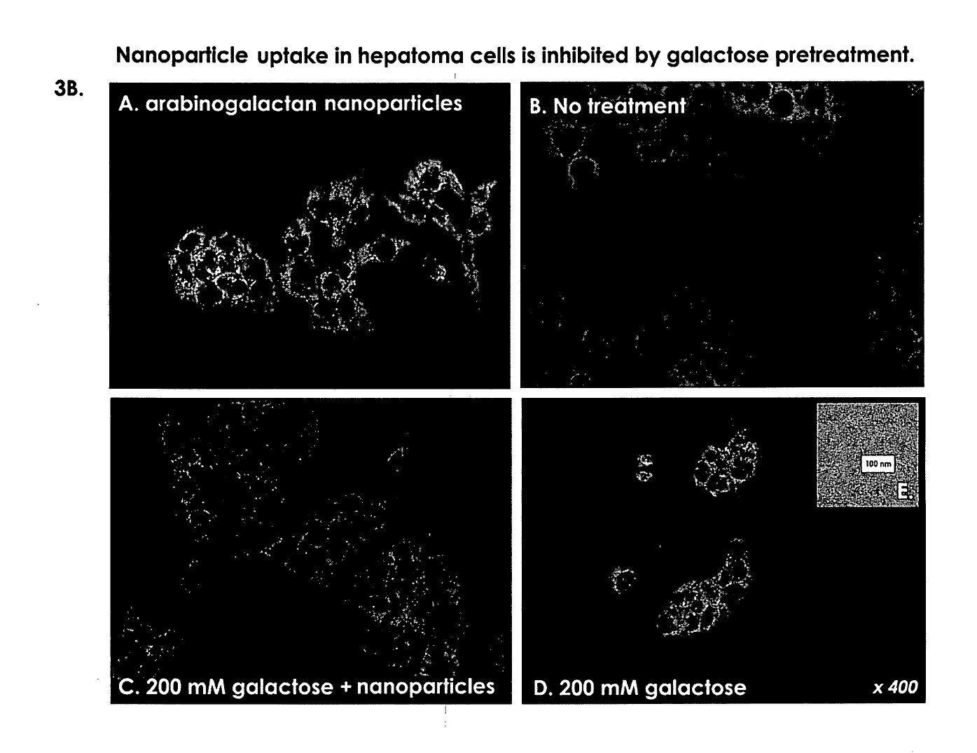

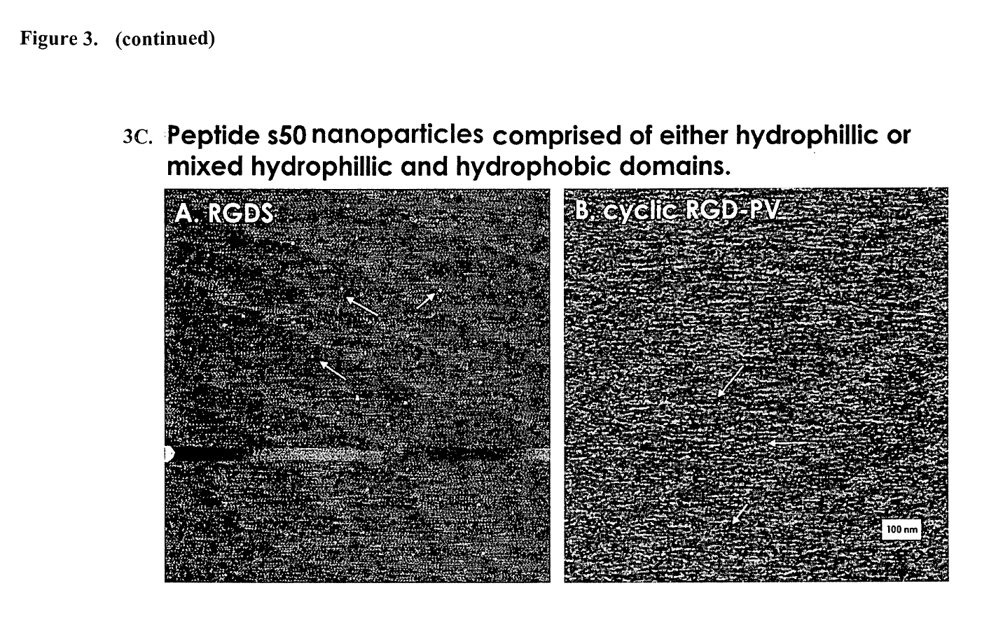

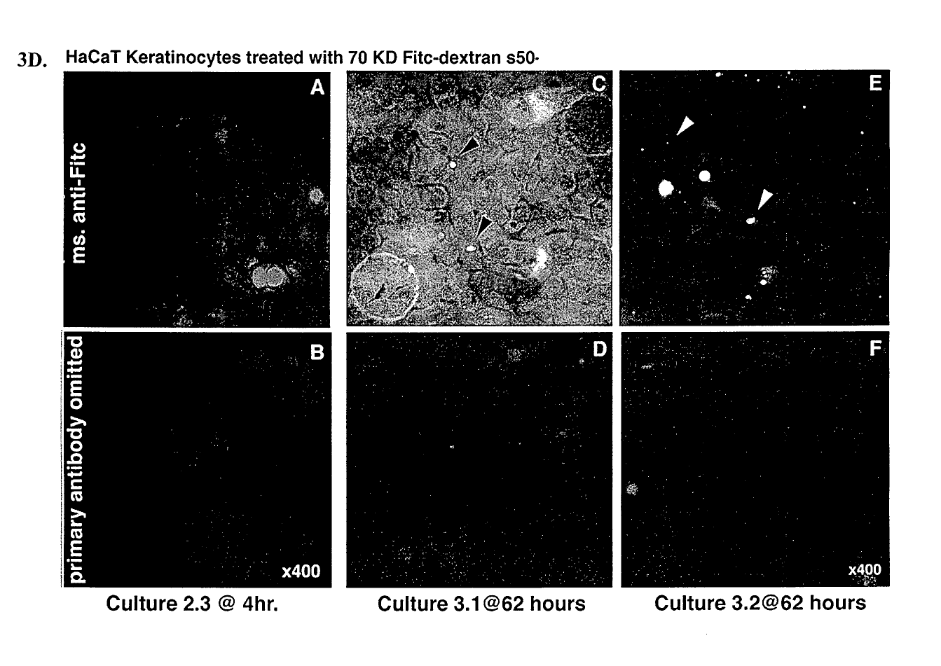

Experiment 3, FIG. 3a-d, shows that cells may be targeted by making nanoparticles, e.g., s50 nanoparticles, by using ligands that bind specifically to cells, including ligands that are specific for cell surface receptors that are internalized via clatharin-coated pits. In this experiment, s50 nanoparticles comprising arabinogalactan were made and directed to human liver cells. The liver cells took up the nanoparticles via receptors specific for arabinogalactan, as was verified using competitive inhibition experiments. Therefore other cell types may be specifically targeted by making nanoparticles having ligands that are specifically bound by cell surface receptors, including cell surface receptors that operate, at least in some situations, via clatharin-pit mediated processes. Further, liver cells may be targeted specifically using arabinogalactan.

As shown in earlier figures in this document, typical sizes for nanoparticles containing plasmid DNA can be in the range of 10 to 25 nm of dry diameter. Such particles should be useful when extracellular delivery of a particle cargo is desired. Some example of such uses would include, for example, delivery of particle cargo on the outside of a cell, especially for delivery of peptides, proteins, sugars and small molecules.

Treatment of Hyperproliferative Disorders

Embodiments include, e.g., nanoparticles targeted to cancerous cells and to cells involved in other hyperproliferative disorders, with the nanoparticles having bioactive, diagnostic, and/or visualization agents. Several experimental treatments for recurrent cancer, e.g., SCCHN, are in later clinical trials or near market approval. They include, for example, INGN 201 (p53 replacement gene therapy delivered by adenovirus), intratumoral Onyx-015 (mutant adenovirus that replicates in p53 −/− cells combined with cisplatin/5-FU) and Erbitux (IMCL C 225, humanized antibody to the EGR receptor). These treatments, however, could all benefit from a better method of delivery e.g., via nanoparticles.

Hyperproliferative disorders may involve genes that ultimately affect gene transcription through their interaction with the DNA scaffold, e.g., histones and chromatin structures. For example, the involvement of nuclear receptors in cancer is documented by mutations in the retinoic acid receptor (RAR), found in acute promyelocytic leukemia (APL), hepatocellular carcinomas and lung cancer. Such alterations may lead to the deregulated recruitment of enzymes having histone deacetylase (HDAC) activity to cause alteration of gene expression. Inhibition of HDACs could thus block gene transcriptional activity and result cellular differentiation of tumor cells, subsequently preventing the cells from further growth or even induce cell death, see also U.S. Patent Ser. No. 60/428,296, filed Nov. 22, 2002.

Numerous examples herein demonstrate the effectiveness of using nanoparticles to deliver agents to cancer cells, including diagnostic, therapeutic, visualization, and bioactive agents. Example 2 shows that cancer cells may be specifically targeted using tenascin, including two types of SSCHN cancer and prostate cancer (Table 4). Tenascin fragments, as well as the whole molecule, are effective for targeting (Table 5). Example 4 shows how antisense against genes active in cancer activity may be delivered to inhibit cancer activities. Example 4 also shows how small molecule toxins, e.g., doxorubicin or cisplatin, may be targeted specifically to cancer cells. The effectiveness of nanoparticles for delivering agents for use in treating minimum residual disease was shown in, e.g., Example 5.

Certain embodiments also provides methods for using probes to detect protein, receptor, or ligand expression in a cell preparation, cell, tissue, or tissue sample. For example, a technique such as in situ hybridization with a nanoparticle directed against a particular cell surface receptor can be used to detect the cell surface molecule in a tissue on a slide (e.g., a tumor tissue). Such probes can be labeled with a variety of markers, including radioactive, chemiluminescent, and fluorescent markers, for example. Alternatively, an immunohistochemistry technique with an anti-protein antibody conjugated to a nanoparticle can be used to detect the protein in a cell or a tissue.

Additional Methods for Administration

Cells and/or tissues may be specifically targeted for many purposes, including for therapeutic, diagnostic, research, and labeling purposes. As already discussed, nanoparticles are described herein that are configured to enter cells via caveolae, a mechanism for cell entry that has many advantages compared to other entry mechanisms. Moreover, such nanoparticles are so small that they penetrate the spaces between cells and move freely through tissues. Indeed, nanoparticles of less than about 70 or 50 nm in diameter are much smaller than the spaces between cells. For example, suitably sized nanoparticles may pass out of blood vessels through the spaces between endothelial cells that line the blood vessels, and into the vascular media. Thus intravascular delivery of suitably sized nanoparticles allows for the nanoparticles to be delivered to tissues beyond the vasculature.

In general, the range of possible targets may be dependent on the route of administration e.g. intravenous or intra-arterial, subcutaneous, intra-peritoneal, intrathecal, intracranial, bronchial, and so forth. For systemic injections, the specificity of this delivery system is affected by the accessibility of the target to blood borne particles, which in turn, is affected by the size range of the particles.

Embodiments include particles with size less than 150 nanometers, which can access the interstitial space by traversing through the fenestrations that line most blood vessel walls. Under such circumstances, the range of cells that can be targeted is extensive. Some non-exhaustive examples of cells that can be targeted includes the parenchymal cells of the liver sinusoids, the fibroblasts of the connective tissues, myofibroblasts, epidermal cells, dermal cells, cells exposed by injury, the cells in the Islets of Langerhans in the pancreas, cardiac myocytes, chief and parietal cells of the intestine, osteocytes and chrondocytes in the bone, chondrocytes in cartilage, keratinocytes, nerve cells of the peripheral nervous system, epithelial cells of the kidney and lung, Sertoli cells of the testis, and so forth.

For subcutaneous injections, the targetable cells includes all cells that reside in the connective tissue (e.g., fibroblasts, mast cells, etc.), Langerhans cells, keratinocytes, and muscle cells. For intrathecal injections, the targetable cells include neurons, glial cells, astrocytes, and blood-brain barrier endothelial cells. For intraperitoneal injection, the targetable cells include the macrophages and neutrophil. Active endothelial transport has been demonstrated for small molecules (transcytosis). Transendothelial migration of macromolecular conjugates and noncovalent paired-ion formulations of drugs and diagnostic agents with sulfated glycosaminoglycan, having a combined size of between about 8000 daltons and about 500 nm are accelerated by the infusion of sulfated glycosaminoglycans (i.e. dermatan sulfate) which become selectively bound to the induced endothelial receptors at sites of disease.

Many aspects of particle delivery are described herein. Delivery of a particle may entail delivery of the particle itself or delivery of the particle as well as structures or compounds that the particle is attached to or associated with. After reading this disclosure, a person of ordinary skill will understand how to adapt methods for using particles that exceed the size for caveolar delivery to the delivery of nanoparticles for caveolar delivery, and how such techniques may used for delivery of larger particles to extracellular sites, tissue, and the like. Delivery techniques used for delivery of particles may, in general, be adapted to use with nanoparticles.

The embodiments include particles delivered by suitable means adapted to the application. Examples of delivery of a particle include via injection, including intravenously, intramuscularly, or subcutaneously, and in a pharmaceutically acceptable solution and sterile vehicles, such as physiological buffers (e.g., saline solution or glucose serum). The particle may also be administered orally or rectally, when they are combined with pharmaceutically acceptable solid or liquid excipients. Particles can also be administered externally, for example, in the form of an aerosol with a suitable vehicle suitable for this mode of administration, for example, nasally. Further, delivery through a catheter or other surgical tubing is possible. Alternative routes include tablets, capsules, and the like, nebulizers for liquid formulations, and inhalers for lyophilized or aerosolized ligands.

Presently known methods for delivering molecules in vivo and in vitro, including small molecules or peptides, may be used for particles. Such methods include use with microspheres, liposomes, other microparticle vehicles or controlled release formulations placed in certain tissues, including blood. Examples of controlled release carriers include semipermeable polymer matrices in the form of shaped articles, e.g., suppositories, or microcapsules. A variety of suitable delivery methods are set forth in, for example, U.S. Pat. Nos. 5,626,877; 5,891,108; 5,972,027; 6,041,252; 6,071,305, 6,074,673; 6,083,996; 6,086,582; 6,086,912; 6,110,498; 6,136,295; 6,142,939; 6,235,313; 6,245,349; 6,251,079; 6,283,947; 6,283,949; 6,287,792; 6,309,375; 6,309,380; 6,309,410; 6,317,629; 6,346,272; 6,350,780; 6,379,382; 6,387,124; 6,387,397 6,416,778 and 6,296,832.

Also contemplated are pharmaceutical compositions and formulations that include a collection of particles or molecules embodied herein. Pharmaceutical compositions containing nanoparticles can be applied topically (e.g., to surgical incisions or diabetic skin ulcers). Formulations for topical administration of nanoparticles include, for example, sterile and non-sterile aqueous solutions, non-aqueous solutions in common solvents such as alcohols, or solutions in liquid or solid oil bases. Such solutions also can contain buffers, diluents and other suitable additives. Formulations for topical administration can include transdermal patches, ointments, lotions, creams, gels, drops, suppositories, sprays, liquids, and powders. Coated prophylactics, gloves and the like also may be useful. Conventional pharmaceutical carriers, aqueous, powder or oily bases, thickeners and the like may be necessary or desirable. Alternatively, pharmaceutical compositions containing nanoparticles can be administered orally or by injection (e.g., by subcutaneous, intradermal, intraperitoneal, or intravenous injection).

For oligonucleotides, examples of pharmaceutically acceptable salts include, e.g., (a) salts formed with cations such as sodium, potassium, ammonium, etc.; (b) acid addition salts formed with inorganic acids, for example, hydrochloric acid, hydrobromic acid (c) salts formed with organic acids e.g., for example, acetic acid, oxalic acid, tartaric acid; and (d) salts formed from elemental anions e.g., chlorine, bromine, and iodine.

In general, for any substance, a pharmaceutically acceptable carrier is a material that is combined with the substance for delivery to an animal. Conventional pharmaceutical carriers, aqueous, powder or oily bases, thickeners and the like may be necessary or desirable. In some cases the carrier is essential for delivery, e.g., to solubilize an insoluble compound for liquid delivery; a buffer for control of the pH of the substance to preserve its activity; or a diluent to prevent loss of the substance in the storage vessel. In other cases, however, the carrier is for convenience, e.g., a liquid for more convenient administration. Pharmaceutically acceptable carriers are used, in general, with a compound so as to make the compound useful for a therapy or as a product.

Nanoparticles may be frozen or reconstituted for later use or may be delivered to a target cell or tissue by such routes of administration as oral, intravenous, subcutaneous, intraperitoneal, intrathecal, intramuscular, inhalational, topical, transdermal, suppository (rectal), pessary (vaginal), intra urethral, intraportal, intrahepatic, intra-arterial, intra-ocular, transtympanic, intratumoral, intrathecal, transmucosal, buccal, or any combination of any of these.

In another application, the nanoparticles may be designed for specific cellular or tissue uptake by polymer selection and/or inclusion of cell-recognition components in a nanoparticle biocompatible polymer shell or coating. Such coatings will have utility for specific or increased delivery of the bioactive agent to the target cell. Alternatively, instead of coating, the cell recognition components may be a component of the nanoparticles. Such applications include, e.g., tumor-targeting of the chemotherapeutic agents or anti-sense DNA, antigen delivery to antigen-presenting cells, ocular delivery of ribozymes to retinal cells, transdermal delivery of protein antibodies, or transtympanic membrane delivery of peptide nucleic acids.

Additional embodiments include peritumoral infiltration techniques, e.g., as described in U.S. Pat. No. 5,945,100. Increased penetration and/or reduced backflow and diversion through the point of entry may be achieved to enhance delivery to a tumor using peritumoral infiltration so that more material is introduced into and remains in the tumor. Such infiltration may be achieve, for example, through the use of a viscous vehicle, most preferably one having a similar density to tissue, for the material to be delivered. Preferred materials include solutions or suspensions of a polymeric material which gel or solidify at the time of or shortly after injection or implantation into or near the tumor. In an embodiment, the solution is injected via a catheter or needle into or near the regions of the tumor to be treated.

Certain embodiments are described in the following Examples, which are intended as illustrations only, since numerous modifications and variations will be apparent to those skilled in the art after reading this disclosure.

### EXAMPLES

Certain of the reagents used were: nucleic acid condensing agents included Poly(ethylenimine) (PEI) at 27 KiloDalton (kD). PEI was typically used at optimized conditions (90% charge neutralization); Polyarginine (parg) at 15,000 molecular weight; Polyornithine (porn) at 15,000 molecular weight; Spemmine (300 MW). Certain of the surfactants used were: 2,4,7,9-tetramethyl-5-decyn-4,7-diol (TM-diol): HLB=4-5. Certain of the polymers used were: Arabinogalactan, food grade, 20,000 MW; Fibronectin, isolated from bovine plasma, F1141, Sigma; Hyaluronan, recombinant, 1 million kiloDalton (MM kD); Povidone (polyvinylpyrrolidone, PVP) 10,000 kD M; Tenascin, 220 kD. Certain expression vectors used were: pT/bsd/bcat 10.6, contains a transposable DNA element for blasticidin resistance and CAT reporter activity, 13.7 kilobases (kB); pEGFP-c3/p57(Kpn/Sma) Clontech enhanced GFP (green fluorescent protein) expression vector modified with a nuclear localization tag from a cyclin dependent kinase to improve microscopy, 4.6 kB. Certain cells were: CRL-1991, human B cell lymphoblasts; Primary human coronary smooth muscle cells, available from Cambrex; HuH7, human hepatoma cell line; Ca9, human tumor cells derived from a squamous cell carcinoma of the gingival; SCC-15, human tumor cells derived from a squamous cell carcinoma of the tongue; Alva-41, human tumor cells derived from a prostate carcinoma metastases.

### Example 1

### Effect of Changing Route of Administration and Tissue Phenotype on Selectivity of Nanoparticle Uptake. Correspondence of Cell Culture Results with Organ Culture Results

The range of usefulness of a synthetic particle material with unknown receptor-binding activity for site-directed targeting of nanoparticles for intracellular uptake was investigated by comparing uptake results in cell culture to uptake results in organ culture. Nanoparticles for uptake and expression studies were manufactured via “dispersion atomization” as described in copending U.S. application Ser. No. 09/796,575, filed Feb. 28, 2001, using a 4.6 kp plasmid expressing Green Fluorescent Protein (GFP, 4297e). Briefly, sub-50 mm diameter nanoparticles as measured by atomic force microscopy of a collection of dried nanoparticles (s50-nanoparticles) were produced by: a) dispersing 200 μg of plasmid complexed with 12 μl of 0.1M PEI into sterile water using a water-insoluble surfactant system of 9.75 μg of TM-diol in 50% DMSO; b) emulsifying the dispersed nucleic acid by sonication with a water-miscible solvent, 150 μl of DMSO; c) inverting emulsion with 750 μl of PBS addition; d) a ligand mixture addition to the hydrophobic micelles, 5 μg of 10,000 MW PVP and adsorption; and e) atomizing ligand-stabilized micelles into a salt receiving solution (200 mM Li⁺, 10 mM Ca²⁺).

Following overnight incubation, particles were collected by centrifugation from the mother liquor for decanting and 0.2 μM filter sterilization. Encapsulation yield was measured at 72% using a standard overnight protein K digestion at 56° C. followed by isobutanol extraction and recovery of DNA on an anionic column. Average particle size was less than 50 nm as measured by tapping mode atomic force microscopy of a 0.1 μg/ml sample dried down on a mica sheet.

For FIG. 1A, 2.5 mcg of PVP nanoparticles were topically applied to organ-cultured pigskin biopsies that had previously (in life) been either irradiated or not using a cobalt source. Following 5 days of culture, biopsies were snapfrozen and detected for GFP expression and location of cells expressing smooth muscle actin. The top row of images are tissues that were exposed to rabbit anti-GFP. The bottom row of images are cells that were exposed to rat anti-human smooth muscle cell antibodies. The left column has images of normal tissue. The middle columns has images of tissue irradiated, and the right column shows the same field of view as the middle column, but shows cell nuclei stained with bisbenzamide. The top left image and top middle images show intense florescence in different areas, indicating that the nanoparticles localized in different ways in radiated versus nonirradiated tissues. The arrows in the right-hand column and middle column indicate cell nuclei.

For FIG. 1B, 10 mcg of nanoparticles comprising PVP and GFP were applied intraarterial to the lumen of a porcine femoral artery ex vivo. Arterial segment was organ-cultured for 5 days before sectioning and detection of GFP expression. The top row shows tissues exposed to the nanoparticles and the bottom row shows control tissues exposed to vehicle only (saline). The left column and middle columns show the same fields of view, with the left column showing florescence imaging of anti-smooth muscle actin and the middle columns showing fluorescence of green florescent protein (GFP). The right column shows fluorescence imaging of GFP using fluorescently labeled antibodies against GFP.

In in vitro cell culture, pvp nanoparticles showed dose-dependent, uniform expression of GFP in both human dermal fibroblasts and human coronary smooth muscle artery cells at about a 1 microgram (mcg or μg) dose of plasmid in an 8 well chamber slide (0.8 cm² per well). FIG. 1A illustrates the nearly 100% efficiency of expression 5 days following treatment. When 2.5 mcg of pvp nanoparticles containing GFP plasmid are topically applied to 8 mm² biopsies of porcine skin, both smooth muscle cells and fibroblasts are transduced in non-irradiated tissue. In irradiated tissue, expression shifts from smooth-muscle cells to smooth-muscle actin positive (sma-+) cells located away from blood vessels. These results are shown in FIG. 1B. The phenotypic shift of fibroblasts into sma-(+) myofibroblasts is a normal feature of wound-healing but persists in the pathobiology of radiation fibrosis and other scarring disease (Martin et. al, (2000), Int. J. Rad. Oncol. Biol. Phys. 47:2 277-90). Porcine skin biopsies were kept alive in organ culture by culturing on a stainless steel mesh in commercially-available organ culture dishes such that the dermis was bathed in culture media but the epidermis kept dry. Biopsies were cultured for 5 to 7 days then snapfrozen for cryosectioning and detection of GFP reporter expression.

10 mcgs of PVP GFP nanoparticles were also applied to the interior of a fresh 3 cm section of porcine femoral artery. The ends of the artery section were clamped shut with sterile paper-binding clips and the artery section incubated with rotation for 30 min. Following incubation, paper-binding clips were cut away, the center section rinsed and cultured for an additional 5 days before snapfreezing in liquid nitrogen, cryosectioning and examination for GFP reporter expression. Results shown in FIG. 1C indicate that the outer section of the artery, the adventitia, is positive for both rat anti human smooth muscle actin antibodies labeled with visualization agents and GFP expression. No GFP expression could be detected in the media or intima of the artery. These results illustrate the capacity of nanoparticles to penetrate into and through an intact endothelial barrier and travel through tissue.

These results also illustrate that for a ligand with an unknown binding profile, e.g. pvp, cell culture studies are sufficient to identify a likely uptake profile in tissue. Further, designed use of regional or localized application for nanoparticles can be used direct nanoparticles past competing cells to the vicinity of target cells.

The strategy of modulating route of administration to expand the utility of a particle material was demonstrated again, this time with a natural, multi-functional ligand material, fibronectin isolated from bovine plasma, as a particle. Particles comprised of fibronectin and containing a GFP expression plasmid were tested in cell culture and organ culture assays as described in the previous set of experiments.

Referring to FIG. 2A, 2.5 mcg of nanoparticles containing nuclear-localized GFP and fibronectin (panel A) or tenascin (panel B) were applied topically to pigskin organ cultures that were cultured essentially as described elsewhere herein. Location of expression was determined by fluorescence microscopy of the GFP after 5 days in culture.

Referring to FIG. 2B, 10 mcg of nanoparticles comprising FN and GFP were applied to the lumen of a porcine femoral artery ex vivo. Arterial segments were organ-cultured for 5 days before sectioning and detection of GFP expression. The top row shows sections treated with nanoparticles and the bottom row shows vehicle-treated sections. The left column shows imaging of GFP and the right column shows imaging of GFP by use of fluorescently labeled antibodies thereto.

Referring to FIG. 2C, 5 mcg of nanoparticles comprising fibronectin (FN) and GFP plasmid were applied to 35 mm cultures of primary hippocampal astrocytes. The left column shows cells that were exposed to the nanoparticles and the right column showed cells that were exposed to control nanoparticles that had GFP plasmid without FN. The top row shows cells that were exposed to fluorescently labeled rabbit-anti-GFP and the bottom row shows the same cells stained with bisbenzamide to visualize the nuclei. The top left panel showed marked fluorescence, indicating that the astrocytes readily took up the nanoparticles comprising FN but not particles without the FN.

Referring to FIG. 2D, s50 nanoparticles comprised of a β-galactosidase reporter gene and either FN, Hyaluronan, or recombinant E-selectin were applied to cultures of 50,000 B cell lymphoblasts and cultured for 3-4 days before detection for beta-galactosidase. These results show that the nanoparticles may be delivered to cells that are in suspension.

Although the cellular distribution of fibronectin's major receptor, the integrin α1β5, is quite broad, it was found that topical administration to epidermis, limited expression to keratinocytes (FIG. 2A), and intraarterial administration ex vivo limited GFP expression to the medial vasculature (FIG. 2B).

Fibronectin particles, like PVP particles, were not limited in tissue penetration by the endothelial barrier and transfection efficiency approached 100%. Primary cell culture transduction studies with rat hippocampal astrocytes indicated that neuronal cultures were also amenable to efficient delivery of macromolecules by ligand-based nanoparticles (FIG. 2C); therefore, FN-decorated particles administered directly into the brain or cerebrospinal fluid (CSF) would be expected to be taken up by astrocytes.

Further, suspension cultures of human B cells were also readily transduced by fibronectin particles indicating usefulness of nanoparticle delivery for ex vivo cultures in suspension or cells of hematopoietic origin (FIG. 2D).

Also shown are B cells transduced with hyaluronan particles and particles comprised of a recombinant E-selectin binding domain. E-selectin is a receptor expressed by activated endothelial cells lining blood vessels during the early stages of inflammation as described in U.S. Pat. No. 5,962,424. White blood cells use E-selectin binding to slow down and exit the blood stream into tissue.

These results demonstrate that particles, e.g., s50 nanoparticles, may be made with ligands for cell surface receptors and thereby targeted to the cells that have the receptors. Since certain cell surface receptors are specific to specific cell types, or are expressed in high numbers relative to other cells, it is possible to target specific cell types by making particles having ligands specific for the receptors that are preferentially expressed by specific cell types. Therefore drugs may be targeted to specific cell types using the nanoparticles, e.g., s50 nanoparticles. Since specific cell types may be targeted, it is possible to rationally design drugs for tissue-specific intracellular delivery of the drugs through caveolar potocytosis. The rationally designed drugs may be designed to achieve specific effects and thereby have a therapeutic effect.

### Example 2

### Contribution of Receptor-Mediated Binding to Intracellular Uptake of Ligand-Based Nanoparticles

It is known that caveolar potocytosis is receptor-mediated, that caveolae are less than about 50 nm at the neck of the vesicle, that caveolae are most likely derived from cholesterol-based microdomains floating on the cell's surface named lipid rafts, that caveolae traffic to locations throughout cells, and that caveolae or similar structures exist in almost every cell in vertebrate systems (Volonte, 1999; Anderson, 1998; Anderson, 1993).

Using a nanoparticle comprising tenascin, it was tested whether extracellular tenascin (at 5 μg/ml in the cell culture media) could inhibit uptake of tenascin s50 nm-nanoparticles that had GFP plasmids and thus inhibit GFP plasmid expression. Cultures were treated with equal amounts of nanoparticles (0.2 mcg DNA/0.8 cm²). Cells were plated into TN media then treated with s50's 24 hrs. later. Following 5 days of culture, cells were fixed, stained for GFP and assessed for nuclear GFP expression by immunofluorescence microscopy. Cells were studied in duplicate wells in 1-2 experiments. Results were quantified by image analysis of colocalized nuclear counterstaining and thresholded image signal. Results are summarized in Table 4 below:

TABLE 4
Extracellular tenascin competes for uptake with tenascin nanoparticles
in carcinoma cells.

| | Percentage cells expressing Green Fluorescent Protein | | | |
|---|---|---|---|---|
| | FN - s50 nanoparticles | | TN - s50 nanoparticles | |
| | | (cultured in | | (cultured in |
| Cell Type | | 5 μg/ml TN) | | 5 μg/ml TN) |
| SSCHN SCC-15 | 43 ± 7 | 63 ± 16 | 64 ± 6 | 8.5 ± 2.6 |
| SSCHN Ca-9-22 | 55 ± 10 | 78 ± 10 | 80 ± 9 | 3.3 ± 2.6 |
| HaCaT | 27 ± 11 | 57 ± 22 | 4.3 ± 1.7 | 13 ± 10 |
| keratinocytes | | | | |
| HDF dermal | 57 ± 15 | 69 ± 7 | 2.3 ± 2 | 16 ± 6 |
| fibroblasts | | | | |
| Alva-41 Prostate | 67 ± 20 | 58 ± 12 | 60 ± 18 | 18 ± 5 |
| Carcinoma | | | | |
| Normal Prostate | 1.6 ± 0.6 | 21 ± 16 | 0 | 0 |

The presence of extracellular tenascin inhibited TN nanoparticle uptake and GFP expression in carcinoma cells but not normal prostate epithelial, immortalized keratinocytes or dermal fibroblasts. In the case of immortalized keratinocytes, GFP expression was increased by TN presence in the media. TN is secreted by keratinocytes during normal dermal wound healing concomitant with upregulation of a migration receptor for TN, αᵥβ₆ Dermal fibroblast also have a wound-healing phenotype (Maragou et. al, Oral Disease, (1996) 20-6). Prolonged exposure to TN in cell culture could induce immortalized keratinocytes to shift to a “wound-healing” phenotype and expression of a TN receptor. SSCHN cells (both SCC-15 and Ca-9-22) exhibit positive signal for αᵥβ₆ integrin in organ culture when separated from the primary tumor. (Unger et al AACR proceedings (2002). In contrast, uptake and expression of FN particles was not affected by tenascin's presence in the cell culture media. Taken together, the data suggests that ligand binding events manipulate ligand-based nanoparticle uptake and phenotypic changes predisposing to said uptake.

Tenascin is a constant feature of reactive stroma surrounding most solid tumors and hyperplastic growth with multiple binding domains for interacting with carcinoma cells (Koukoulis, 1993). It was tested whether the full protein was required for nanoparticle uptake rather than smaller segments. This requirement was examined by comparing the particles made of different TN protein domains for carcinoma drug delivery of an antiproliferative antisense. TN protein domains are described in detail in Aukhill et al., J. Biol. Chem. (1993).

TABLE 5

| | | IC₅₀ for growth inhibition of |
|---|---|---|
| | | particle bearing phosphodiester |
| | | antisense to Casein Kinase 2 (% |
| Protein segment in particle | Description | of matched Cisplatin IC₅₀) |
| Entire protein - isolated | All binding sites including | IC₅₀ for growth inhibition of |
| from cell culture | EGF domains | capsule bearing antisense to |
| supernatant of glioma | | Casein Kinase 2 (% of matched |
| cells. | | Cisplatin IC₅₀, molar basis) |
| TnFnall | Fibronectin domains only | 10% (phosphodiester chimeric) |
| TnFbgn | Fibrinogen domain includes at | 6.5% (phosphodiester) |
| | least αᵥβ₃ and proteoglycan | |
| | binding sites | |

Particles made of tenascin subdomains showed activity equivalent to the whole protein and were effective for delivery of antisense to carcinoma cells. These results show that cell targeting/recognition strategies identified and developed using nanoparticles, using whole molecules, subdomains or peptide mimetics, will be at least as effective as conventional drug targeting technologies, e.g. bioconjugation, agents delivered using fusion proteins, or as a component in any particle assembly for cell-specific delivery.

Tenascin's role as matrix molecule in wound healing predicts that tenascin may have a useful role for therapeutic delivery of molecules in other pathophysiologies where normal wound healing is characterized by overproliferation, scarring or hyperplastic growth. This hypothesis was tested by comparing the effect of “scrape-wounding” monolayer cultures of human coronary artery smooth muscle cells on uptake TN nanoparticles bearing GFP plasmid.

FIG. 3A shows Tenascin/GFP nanoparticle uptake in in vitro smooth muscle cells±scrapewounding, with 3AA and 3AA′ showing the same field of view of non-scraped cells, with 3AA being a phase contrast image showing cells and 3AA′ being a fluorescence image showing GFP florescence. FIGS. 3AC and 3AC′ show the same field of view of non-scraped cells, with 3AC being a phase contrast image showing cells and 3AC′ being a fluorescence image showing GFP florescence. Both 3AA and 3AC show multiple cells. FIG. 3AA′ shows cells that have not been wounded or exposed to nanoparticles; FIG. 3AC′ shows cells that have not been wounded, but have been exposed to tenascin-GFP nanoparticles: no fluorescence is visible.

It was found that scrape-wounded cultures were stimulated to take up TN particles and show GFP expression following 5 days in culture (FIG. 3A). A 30 mer peptide (peptide VIII) has been mapped to the αᵥβ₃ site in the fibrinogen domain of TN that stimulates migration in smooth muscle cells. This peptide and others are described in U.S. Pat. No. 6,124,260 and incorporated herein. Nanoparticles of tenascin, tenascin subdomains or peptides mimicking binding domains are expected to be useful for delivery of therapeutic in proliferative disorders.

It was next examined if known uptake by a ligand via clathrin-coated pit receptor-mediated endocytosis precluded the use of that ligand as a particle material in ligand-based nanoparticles undergoing caveolar potocytosis. FIG. 3B shows uptake by adherent HUH7 hepatoma cells of nanoparticles comprising 14 kb transposons and arabinogalactan. Cells were cultured in 8-well chamber slides and treated for 15 hours. Fluorescence detection was performed by using fluorescent antibodies to detecting for anti-sheep IgG against sheep IgG present in the particle. The left column shows cells exposed to 1 mcg of the nanoparticles, and the bottom row shows cells exposed to 200 mM galactose. The top right panel shows cells that were untreated. Subpanel e is AFM micrograph nanoparticle containing the 13.7 Kb plasmid, showing that the nanoparticles are about 15-20 nm in approximate diameter. Nanoparticles were taken up by the cells (top left panel), but uptake was blocked by competitive inhibition using excess galactose (bottom left panel).

Arabinogalactan, a sialylated, galactose-terminated carbohydrate derived from larch trees, has been used to direct superparamagnetic metallic oxides to the liver via direct conjugation. Uptake into liver hepatocytes is believed to be mediated by the asialoglycoprotein receptor and is described in U.S. Pat. No. 5,284,646. Unlike biological materials, uptake by clathrin-coated pits and eventual localization in lysosomes does not preclude usefulness for magnetic diagnostic imaging agents. In U.S. Pat. No. 5,679,323, the participation of arabinogalactan in receptor-mediated endocytosis terminating in lysosomes of hepatocytes and its usefulness because of this for delivery of imaging agents is described.

Nanoparticles of arabinogalactan were manufactured as described in Example 1 except that 6.5 mcg of arabinogalactan were added to 250 mcg of a 13.7 kb plasmid (pT/bsd/bcat 10.6) condensed with 11 μl of 0.1 M PEI (21413L). A small amount (1% of coating weight) of sheep IgG was “spiked” into the arabinogalactan to enable immunodetection of nanoparticles uptake by anti-sheep IgG antibodies. Nanoparticles were on average 11±2 nm in diameter by tapping mode atomic force microscopy (FIG. 3B, view e). Nanoparticle uptake into human hepatoma cells was examined by treating HUH7 hepatoma cells, plated on chicken tenascin, overnight with 0.5-2 mcg/0.8 cm², fixing with 2% paraformaldehyde and immunodetecting for nanoparticles by anti-sheep antibodies. Sensitivity to the asialoglycoprotein receptor was tested by pretreating cells and then coincubating with 100 to 200 mM galactose to compete off potential nanoparticle uptake. We found that, after 15 hours of incubation, nanoparticles were moving into the nucleus from caveolae located at the surface of the cell, one of several recognizable patterns of nanoparticle uptake in vitro (FIG. 3B, a vs. b). Coapplication 200 mM galactose blocked appearance of nanoparticles in the nuclei of the hepatoma cells (FIG. 3B, c vs. d). Examples of compositions for directing nanoparticle delivery are provided above, e.g., in Tables 1 and 2.

It was next examined whether any limitations existed with respect to peptide design in the context of nanoparticle process chemistry by manufacturing particles using either the fully hydrophilic peptide RGDS or the mixed hydrophilic/hydrophobic domain peptide RGD-PV. FIG. 3C shows AFM tapping-mode micrographs of nanoparticles comprising 5 kb luciferase expression vector and RGDS or cyclic RGD-PV. Nanoparticles were successfully made using either peptide. Particles were manufactured as described in Example 1, except that a commercially prepared luciferase expression plasmid of about 5 kb was used (21411J, 12K). AFM micrographs indicate that the hydrophillic peptide produced a slightly larger particle, but that both peptides produce nanoparticles well under an average dry diameter of 50 nm (rgds vs. rgd-pv: 13±2 vs. 10±2 nm, (FIG. 3C). Peptides containing hydrophobic domains have been problematic due to issues deriving from aggregation of hydrophobic domains in aqueous systems (Lackey et. al, 2002, Bioconjugate Chem. 13, 996-1001). However, most peptides can be successfully used in a nanoparticle structure as described herein.

Further, it was examined whether intracellular delivery by ligand-based nanoparticles was limited to the nucleus of the target cell by following the fate of fluorescently labeled 77 kD dextran. FIG. 3D shows HaCaT keratinocytes treated with 70 kD FITC-dextran s50-nanoparticles. Labeled dextran was nanoencapsulated using hyaluronan (1 MM KD) as described. Nanoparticles were sized at 26±11 nm (mean, SD) by AFM. 15 mcg of s50-NC dextran was added to serum-containing culture media with stirring and cultures were incubated until fixation time. Dextran location was detected by monoclonal antibody complexes labeled with Cy2. Images were collected on either a Zeiss Axioplan or Olympus fluorescence microscope. Omission controls are included to control for different light conditions on the two microscopes used. (subpanels A, B) After 4 hours of incubation, what signal is detectable is located in the keratinocyte nuclei. Transit time for s50-nanoparticles to the nucleus varies from 2 to 18 hours by cell type and is tracked by detection of Sheep IgG added to the protein coat during preparation. (subpanels C, D & E, F). By 62 hours, FITC-dextrans have moved from cell nuclei to the cytoplasm (subpanels C). Bright spots (highlighted by arrows in subpanels C, E) have been shown in multiple separate experiments to colocalize with Lamp-1, a lysosomal marker, suggesting that transported dextran may traffic from the cytoplasm to the lysosomes with some heterogeneity in kinetics between individual cultures.

Fluorescein isothiocyanate (FITC)-dextran was packaged in a nanoparticle with hyaluronan (1 MM kD) essentially as described in Example 1 with the following changes; 100 mcg of dextran in 20 μl of water was dispersed in 7 mcg of TM-diol, followed by the addition of 2 mcg of hyaluronan (120413f). Particles were sized at 26±11 nm by tapping mode AFM as described. 15 mcg of nanoparticles having FITC-dextran was added to serum-containing culture media with stirring and cultures were incubated until fixation time. Dextran location was detected by monoclonal antibody complexes against dextran labeled with the visualization agent Cy2. Images were collected on either a Zeiss Axioplan or Olympus fluorescence microscope. Omission controls are included to control for different light conditions on the two microscopes used (A, B). After 4 hours of incubation, what signal is detectable is located in the keratinocyte nuclei. Transit time for s50-nanoparticles to the nucleus varies from 2 to 18 hours by cell type and is tracked by detection of Sheep IgG added to the protein coat during preparation. (C, D & E, F). By 62 hours, FITC-dextrans have moved from cell nuclei to the cytoplasm (C). Bright spots (highlighted by arrows in C, E) have been shown in multiple separate experiments to colocalize with Lamp-1, a lysosomal marker, suggesting that transported dextran may traffic from the cytoplasm to the lysosomes with some heterogeneity in kinetics between individual cultures.

### Example 3

### Extracellular Delivery by Ligand-Based Ultrasmall Particles

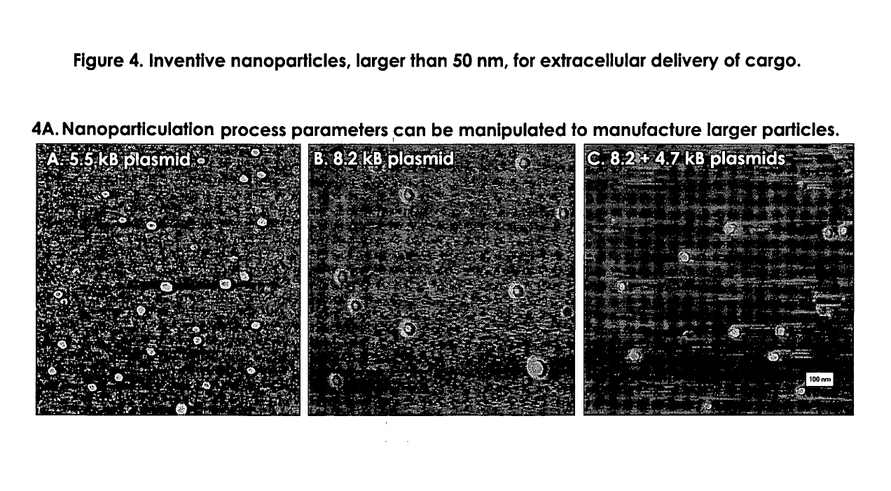

Large, uniform particles may also by made as described in Example 1, but instead of incubating in a salt solution overnight at 4° C., salt solutions containing particles are incubated for longer periods of time. Such particles are illustrated in FIG. 4A, which shows AFM tapping mode micrographs of nanoparticles made with various sized plasmids. The following table shows characterization results for the illustrated nanoparticles of FIG. 4, manufactured with a double coatweight and incubated for 56 hours in a salt solution.

TABLE 6
Larger nanoparticles, useful for a extracellular delivery

| | | | Dry diameter, | Uptake, overnight in |
|---|---|---|---|---|
| Formula | Plasmid size | | nm | Rat-1 fibroblasts |
| 6245G | 5.5 | kilobases | 36 ± 8 | good |
| 6249K | 8.2 | kilobases | 49 ± 10 | poor |
| 62410L | 8.2 and 4.7 | kilobases | 53 ± 8 | none |

Nanoparticles with plasmids as shown elsewhere herein were made with about 10-25 nm diameter, but, as shown in Table 6, may also be made in larger sizes. Cells are expected to not take up relatively large particles so that delivery to tissues and cells without cellular uptake may be accomplished.

### Example 4

### Ligand-Based Nanoparticles for Enhanced Delivery of Anti-Tumor Compounds, Particularly Antisense Compounds to the Casein Kinase 2 Molecule

After demonstrating the usefulness of ligand-based nanoparticles for site-specific delivery of functioning genes, the usefulness of the inventive nanoparticles for effective delivery of antisense and small molecules was examined. The difficult problem of drug delivery into solid tumors was studied, using the critical regulatory enzyme Casein Kinase 2 (CK2 or PKC CK2) as our model molecular target and cisplatin as a model small molecule drug.

Tenascin nanoparticles were prepared for functional growth inhibition studies by dispersion atomization as described in Example 1 using a 20 mer phosphodiester sequence spanning the translation start site of the alpha subdomain of CK2 (PO, 11207p, (Pepperkok, 1991). In brief, s50-nanoparticles were produced by: a) dispersing 200 μg of antisense DNA oligonucleotide complexed with 60 mcg of 15K MW polyornithine into sterile water using a water-insoluble surfactant system of 8 μg of TM-diol in 50% DMSO; b) emulsifying the dispersed nucleic acid by sonication with a water-miscible solvent, 150 μl of DMSO; c) inverting emulsion with 750 μl of PBS addition; d) “coating” hydrophobic micelles by ligand mixture addition, 10 μg of 225 Kd tenascin and adsorption; and e) atomizing ligand-stabilized micelles into a salt receiving solution (200 mM Li⁺, 10 mM Ca²⁺). Following overnight incubation, particles are collected by centrifugation from the mother liquor for decanting and 0.2 μM filter sterilization. Encapsulation yield was measured at 74% using a standard overnight protein K digestion at 56° C. followed by isobutanol extraction and recovery of DNA on an anionic column. Average particle size was less than 50 nm as measured by tapping mode atomic force microscopy of a 0.1 μg/ml sample dried down on a mica sheet.

Antisense nanoparticles were compared to liposomal particles using published methods for liposomal delivery of phosphodiester antisense to head neck cancer cells (SSCHN Ca-9-22) in vitro (Faust et. al, Head Neck (2000), 22:341-6. In these studies, 96 well plates were seeded at 2000 cells per wells pretreated with tenascin, incubated for 72 hours, and observed to have an IC₅₀ for growth inhibition at 40 μg/ml (6 μM). FIG. 5A shows a growth inhibition curve comparing nanoparticles to liposomes. FIG. 5A shows the survival of Ca-9 SCCHN tumors after exposure to: s50 nanoparticles loaded with FITC and phosphodiester antisense against CK2α (SEQ ID NO 1, FITC-sense) or a sense sequence of CK2α (complement to SEQ ID NO 1, FITC-sense); or exposure to liposomes loaded with DOTAP liposomal transfection reagent and CK2α antisense (SEQ ID NO 1, DOTAP antisense) or CK2α sense (complement to SEQ ID NO 1, DOTAP sense) or a scrambled CK2α antisense (DOTAP antisense). DOTAP is commonly used for transfection of DNA into eukaryotic cells for transient or stable gene expression. Half-maximal specific growth inhibition was not reached for the liposomal antisense formulations, but 250 nanoparticle antisense formulations did achieve a greater than half maximal performance. Further, liposomal formulations for antisense, sense, and control sequences were comparable in their effects, but s50 nanoparticle antisense was much more effective than the sense sequence (FIG. 5A). Thus it may be concluded that nanoparticles delivered functional antisense sequences to tumor cells.

Next, a number of different medicinal chemistry formats or backbone chemistries were compared in the s50 nanoparticle format. An important issue in design of antisense molecules, to date, has been balancing binding affinity for the target mRNA with ensuring sufficient stability from 3-prime exonucleases in the extracellular and intracellular spaces. Binding affinity and thus one mode of antisense inhibition of protein translation is typically improved by native, particularly RNA structures. Native DNA regions also provide additional mode for antisense activity by creating a site for RNAse H activity. Nuclease resistance has traditionally been designed into antisense molecules by manipulating the side chains or linkages of the oligonucleotide to delay or block nuclease activity and the demise of the therapeutic molecule. However, this increase in nuclease resistance has generally occurred at the cost of decrease in desirable binding affinity.

Using the same sequence, the alternative antisense chemistries were formulated as described for the phosphodiester antisense 20mer against CK2α, above, with the substitution of 200 μg of spermine as a cationic condenser for the molecules containing RNA. The morpholino and duplex RNA sequences were different but derived by standard means. A chemically synthesized small-interfering RNA candidate was formulated using alternative CK2 sequences and compared to a nanoencapsulated cisplatin formulation. These formulas were assembled in manner like the phosphodiester with the substitution of 200 μg of spermine, 70 μg of 15K MW polyarginine and no condenser respectively for the molecules. Sequences for these alternative molecules are listed in Table 9.

Antisense molecules were tested for growth inhibition against the chemoresistant head neck cancer cell line SCC-15 at 10,000 cells per well, the cells being pretreated with tenascin, with results as shown in FIG. 5B-C. Referring to FIG. 5B, PO refers to phosphodiester antisense referred to as asCK2 in Table 9 (SEQ ID NO 1), PO sense refers to phosphodiester sense sequence complementary to asCK2, siRNA refers to a duplex RNA sequence that is screened from the asCk2 sequence, 2OME RNA refers to a nucleic acid of the sequence SEQ ID NO 1 that is all RNA and is al methylated, and PO RNA refers to a proprietary chimeric molecule having the sequence of SEQ ID NO 1 but being a mixture of RNA and DNA and having phosphodiester and 2OME backbone. Molecules containing a phosphorothioate backbone were formulated and found to have performance similar to 2OME RNA. All antisense formulas showed activity with variation in apparent pharmacokinetics. IC₅₀ 's for these formulas for growth inhibition ranged from 3 μM for the PO RNA chimeric to at about 20 μM for the duplex-RNA molecule. Because of it's capacity for being metabolized, the PO RNA construct within the context of a colloidal formulation will offer advantages in safety for delivery of cytotoxic constructs.

Referring to FIG. 5C, cisplatin TN/x s-50 refers to nanoparticles comprising cisplatin and a 1:1 w/w ratio of tenascin: dextran. Tn s-50 refers to nanoparticles comprising cisplatin and tenascin, asCK2 TN s-50 refers to nanoparticles comprising tenascin and asCK2 antisense of sequence SEQ ID NO 1, and free cisplatin refers to cisplatin added to the cell medium. The nanoparticles comprising cisplatin increased overall in vitro kill from zero to about 20%, indicating that the nanoparticle vehicle was increasing the amount of productive drug entry into the cell. Nanoencapsulated doxorubicin (not shown) had an IC₅₀ of 15% of that of cisplatin in the SCC-15 head neck line.

The nanoencapsulated phosphodiester antisense formula referred to as asCK2 in Table 9 was also tested in hormone-insensitive PC3 cells and hormone-sensitive Alva-41 prostate carcinoma cells in vitro; IC₅₀'s for growth inhibition were 40 μM (65% of cisplatin's IC₅₀) and 15 μM, respectively (data not shown). In these studies, cells were seeded at 5,000 cells per untreated well. Thus it may be concluded that multiple antisense chemistries showed increased effectiveness following their incorporation into specifically targeted addition of nanoparticles.

Cisplatin was nanoencapsulated into the various candidate tumor binding agents as described previously and nanoparticles were compared for growth inhibition in a metastatic variant of Alva-41 prostate carcinoma cells and Ca-9-22. Formulas were tested in duplicate in two separate experiments. Results are illustrated for the prostate cell line in FIG. 5D. Referring to FIG. 5D, PEX-MMP-1/Cisplatin refers to s50 nanoparticles comprising cisplatin and the Recombinant Pex binding domain of membrane-associated Matrix Metalloproteinase-1 (see Bello et. al, Cancer Research (2001) 61: 8730-36); Tenascin/Cisplatin refers to s50 nanoparticles having tenascin and cisplatin, FN-PHSCN/Cisplatin refers to nanoparticles comprising the FN-PHSCN fragment and cisplatin, Osteonecetin/asCK2 refers to s50 nanoparticles comprising osteonectin and the asCK2 antisense sequence, galectin-3/cisplatin refers to s50 nanoparticles comprising galectin-3 and cisplatin, hyaluronan/cisplatin refers to s50 nanoparticles comprising hyaluronan and cisplatin, and naked cisplatin refers to the addition of free cisplatin to the cell medium. In these experiments cells were plated at 5,000 per well and followed for 72 hours. IC₅₀'s for growth inhibition ranged from 60 μM to 200 μM for the nanoencapsulated cisplatins compared to 100 μM for free cisplatin. As a comparison, based on a standard male patient, an acceptable in vitro dose of cisplatin would correspond to about 10 μg/ml or 30 μM. Given the reasonable expectation of a 10 to 100-fold increase in maximum tolerated dose by targeted delivery, any of these particles could reasonably be considered for additional pharmaceutical development. In the Ca 9-22 head neck line, both tenascin and osteonectin showed growth inhibition activity. This data shows that numerous types of molecules, regardless of their structure but, with consideration of their role in cell pathobiology, can be usefully nanoencapsulated in multiple appropriate components to exhibit broad anti-tumor activity.

### Example 5

### Effectiveness of Nanoencapsulated Compounds Against Tumor Nests in Organ Culture

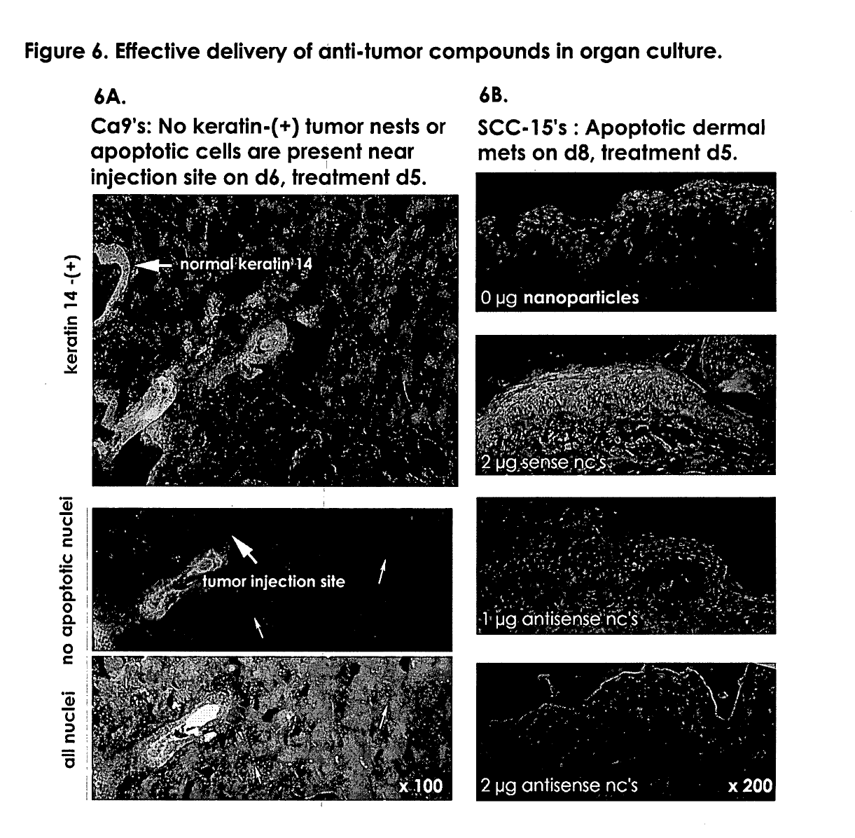

To confirm the in vitro biological activity of nanoencapsulated anti-tumor compounds, 3 formulations were tested against 3-D in vitro tumor nests grown in pig dermis organ culture, see FIG. 6. The three compounds were nanoparticles comprising Tenascin and phosphodiester antisense CK2α having a sequence of SEQ ID NO 1; nanoparticles comprising truncated Galectin-3 and CK2α phosphodiester antisense of SEQ ID NO 1 and nanoparticles comprising Hyaluronan and cisplatin. Porcine skin biopsies (8 mm diameter), were either injected or not with carcinoma cells and cultured in duplicate at an air-water interface on a 300 μm stainless steel mesh in commercially available organ culture dishes. At 0.5 to 3 days post injection, biopsies were treated topically with nanoencapsulated phosphodiester antisense to casein kinase 2 alpha, a small molecule anti-tumor agent or buffer, then organ-cultured for 3 days. Tumor-bearing biopsies were snapfrozen in liquid nitrogen, then cryosectioned into 6 micron sections for tumor detection using immunofluorescence microscopy. Tumors were detected by either immunosignal for keratin 14 (K-14, SSCHN), prostate-specific antigen (psa, prostate carcinoma), or apoptosis via the TUNL method. Descriptive results are summarized in the following Table 8 and results for the head neck cancer lines are depicted in FIG. 6.

TABLE 8
Efficacy of nanoencapsulated compounds in model of minimum residual disease.

| | | | | | |
|---|---|---|---|---|---|
| | Cells | Time lag | | Time lag | |
| | injected | between | | between | |
| | into | tumor | Tumor | tumor | |
| | porcine | injection | nest | injection | Tumor nest |
| Tumor nest | skin | and | starting | and | description at |
| (dose/molecule/particle) | biopsy | treatment | description | termination | termination |

SSCHN Ca-9-22 psg. 28, p6F1

| | | | | | |
|---|---|---|---|---|---|
| 0 μg | 200,000 | NA | NA | 5 days | Primary tumor |
| | | | | | along injection, |
| | | | | | scattered nests |
| | | | | | throughout |
| | | | | | biopsy |
| 2 μg antisense TN | 200,000 | 18 hours | NA | 5 days | none |

SSCHN SCC-15, psg. 4, p26F1

| | | | | | |
|---|---|---|---|---|---|
| 0 μg | 200,000 | NA | mm, | 8 days | Primary tumor |
| | | | CK2-(+), | | along injection, |
| | | | K-14-(+) | | diffuse cell |
| | | | αᵥβ₆-(+) | | groups |
| | | | αᵥβ₃-(+) | | throughout |
| | | | | | biopsy, complete |
| | | | | | colonization of |
| | | | | | epidermis |
| 0.5 μg antisense TN | 200,000 | 3 days | | 8 days | 400 μm primary |
| | | | | | tumor nest, |
| | | | | | epidermis |
| 1 μg antisense TN | 200,000 | 3 days | | 8 days | Still present |
| | | | | | epidermis |
| 2 μg sense TN | 200,000 | 3 days | | 8 days | Possibly |
| | | | | | increased |
| | | | | | epidermal colonization, |
| | | | | | αᵥβ₆- |
| | | | | | (+) and apoptotic |
| | | | | | by TUNL |
| 2 μg antisense TN | 200,000 | 3 days | | 8 days | No tumor cells |
| | | | | | by K-14 |
| | | | | | detection |

Prostate Carcinoma Alva-41, psg. 371, p33F3

| | | | | | |
|---|---|---|---|---|---|
| 0 μg | 200 | 3 days - | 50 μm nest | 5 days | Biopsy was dead - |
| | | couldn't | plus | | a problem with |
| | | find | primary | | tumor |
| | | injection | | | overgrowth |
| | | site | | | |
| 5 μg antisense | 200 | | | 5 days | Biopsy alive, no |
| recombinant galectin 3 | | | | | tumor by PSA at |
| (rtG3) | | | | | i.site |
| 50 μg antisense rtG3 | 200 | | | 5 days | Biopsy alive, no |
| | | | | | tumor |
| 5 μg cisplatin HA | 200 | | | 5 days | Biopsy was dead, |
| | | | | | few scattered |
| | | | | | living carcinoma |
| | | | | | cells |
| 50 μg cisplatin HA | 200 | | | 5 days | Biopsy alive, but |
| | | | | | epidermis |
| | | | | | appears PSA-(+). |

It may be concluded from these results that nanoencapsulated compounds, especially antisense, showed excellent anti-tumor activity in a reasonable model of minimum residual disease. Minimum residual disease refers to small nests of tumor left behind following surgical removal of the primary tumor or in the bloodstream following chemotherapy, but have not recruited an independent blood supply.

### Example 5

### Usefulness of Nanoencapsulated Antisense to CK2α for Anti-Tumor Treatment in an Animal Model of Human Cancer

It was tested whether nanoencapsulated phosphodiester antisense to CK2α showed biological activity in vivo using 2 mice, one treated topically and the other by injection. Nude mice were injected dorsally with 2e6 SSC-15 cells and treatment began when tumors were palpable (3×4 mm). Tumor growth in an untreated mouse resembled that of the mouse that received intratumoral nanoparticle antisense (83.5 mm³ in 7 days). FIG. 7 shows that topical treatment was more effective than intratumoral injection in regressing the nude mouse xenograft.

Essentially, it was found that 3 small (10-30 μg) topical repeat doses resulted in 10 apparent tumor free days and that 5 small doses followed by one big (200 μg) dose resulted in regression combined with massive edema and transient inflammation at the site. Mice were treated topically by applying sequential 50 μl aliquots for 5 minutes each. In contrast, we found that 1 small intratumoral injection induced 3 tumor free days and that subsequent groups of small injections induced 1 then no tumor free days. A final large injection (200 μg) was followed by rapid tumor growth. The 200 μg dose level was chosen as being below the typical dose (20 mg/kg) where hematological toxicities appear in mice treated with nuclease-resistant phosphorothioate with repeat i.v. administration (Cooke). Both tumors were 3×4 mm at the time of treatment with the 200 μl dose. Blood work executed at time of sacrifice indicated normal CBC's for the injected mouse and slight elevation in neutrophils in the topical mouse consistent with a mild inflammatory state.

At sacrifice, the tumor from the topically treated mouse appeared hemorrhagic and necrotic while the i.t. tumor was enveloped in a whitish, fibrous capsule. Residual tissue in the topical mouse was centered around the feeder blood vessel. Tumors are pictured in FIG. 7 inset. The diameter of the mass from the topical mouse is approximately 2 mm compared to 6 mm for the mass from the i.t. mouse. Significantly, a nearly linear correspondence was observed between the 2 μg of nanoparticle required to treat a 0.8 mm (0.256 mm³) tumor nest in a pigskin biopsy and the 200 μg required to treat 3.5 mm tumor (18 mm³) in a mouse. This correspondence confirms the view that our pigskin model is a relevant model of minimal residual disease and is consistent with the uniform delivery of antisense required to kill every tumor cell.

It was tested whether Asnan (i.e., s50 nanoparticles comprising SEQ ID NO 1 and tenascin) induced carcinoma death in vivo by apoptosis by examining immunofluorescent staining of activated Caspase 3 (aC3), an early marker of apoptosis, in center sections from the excised tumors. In general, the topically-treated tumor was characterized by complete internal necrosis, surrounded by an extensive stratified capsule. In the injected tumor, aC3 signal was concentrated in the needle track, but distributed out evenly from the track suggesting tumor penetration with the delivery needle did occur, but inadequate amounts of drug were delivered to carcinoma cells. In contrast to the topically-treated tumor, the injected tumor exhibited occasional regions of capsule stratification and pockets of apoptotic cells by both TUNL staining for fragmented DNA and positive aC3 signal. Given that increased intratumoral hydrostatic pressure decreases rapidly at the margin of solid tumors (reviewed in Jain et al., Sci. American (1994) 7:58-65), we concluded that topically delivered nanoparticles may more effectively distribute drug into a solid tumor. Potentially, a uniform, peripheral kill could break down the pressure gradient and resistance to drug distribution. An additional probable mode of action is that early death of the more active, invading front of a tumor may result in a more complete kill due to the dependence of weaker, interior cells on peripheral cells for survival signals (Gapany et al., 1995; Tawfic et al., 2001). It may be concluded that “peritumoral” application of therapeutics can offer advantages in treating solid tumors.

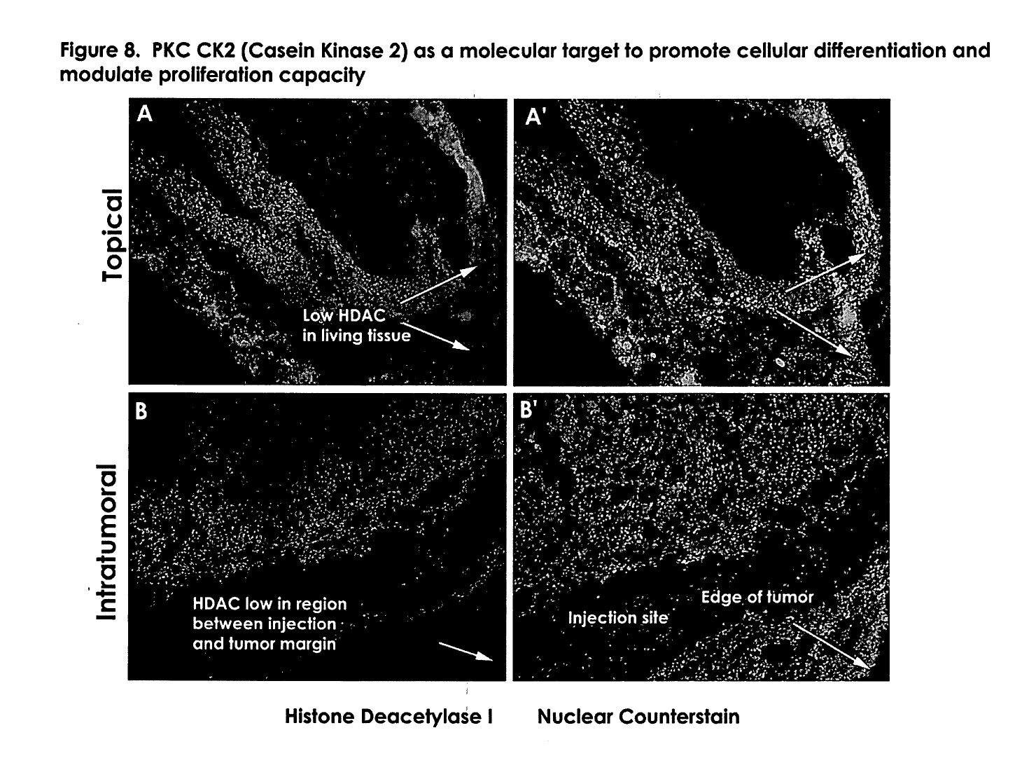

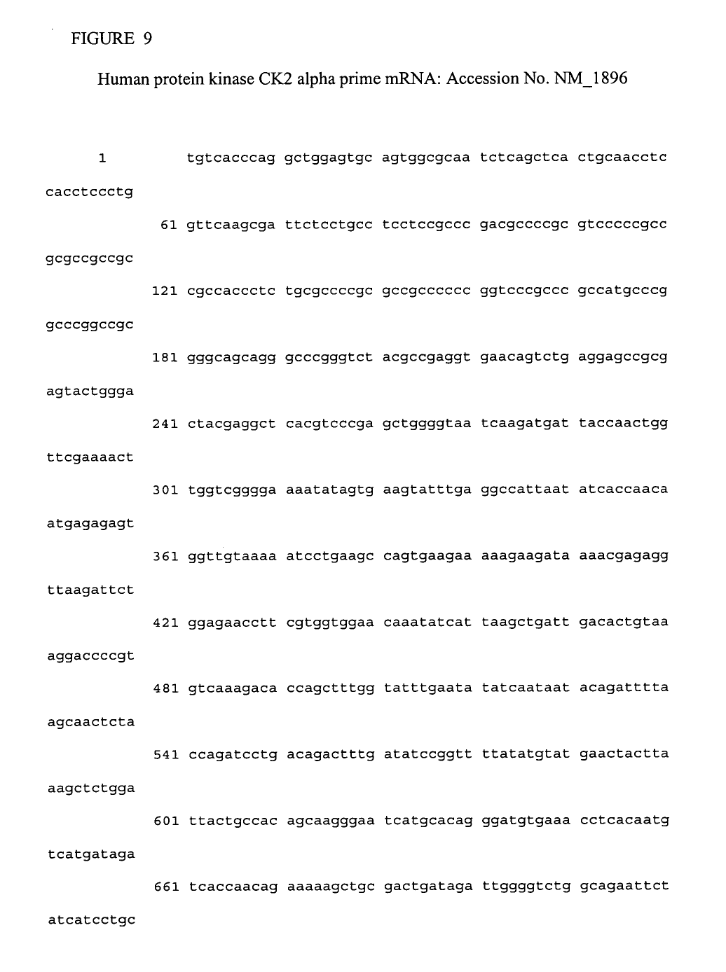

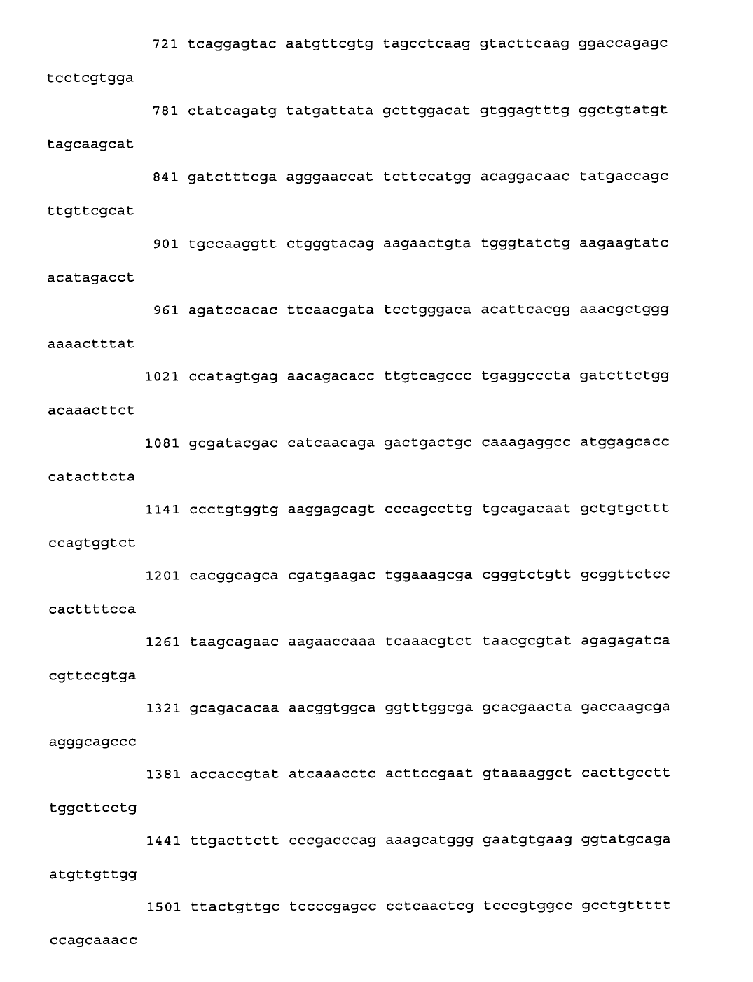

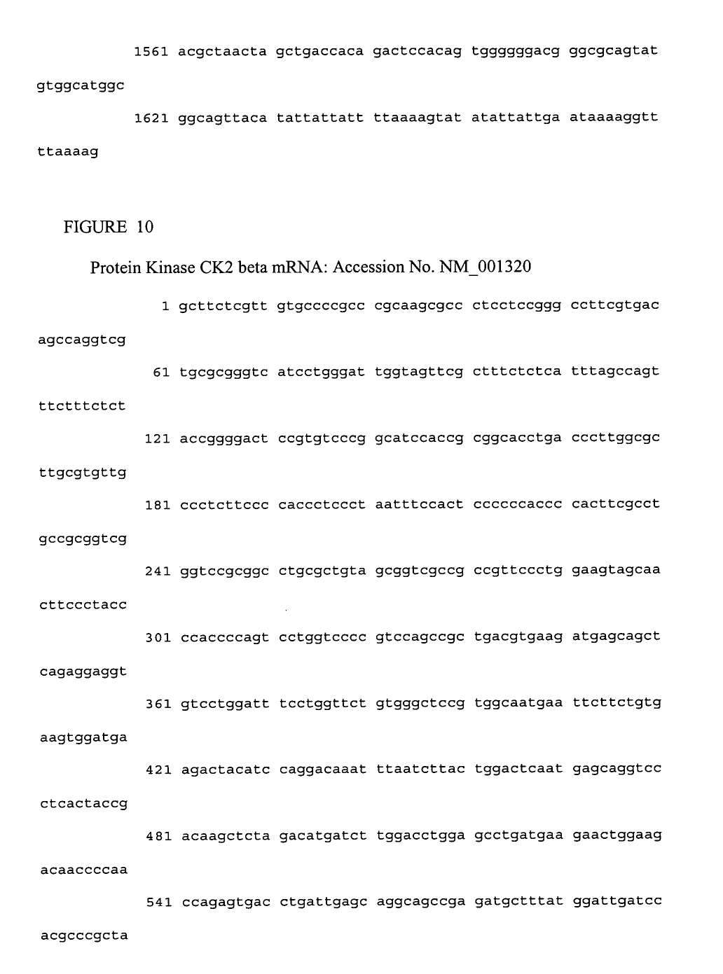

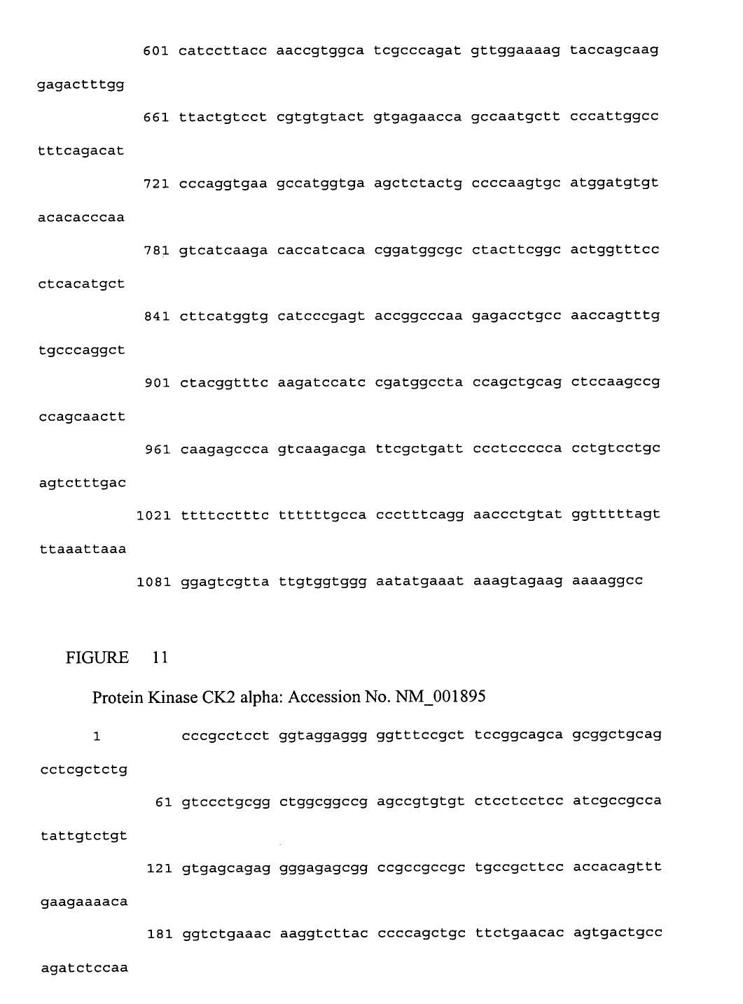

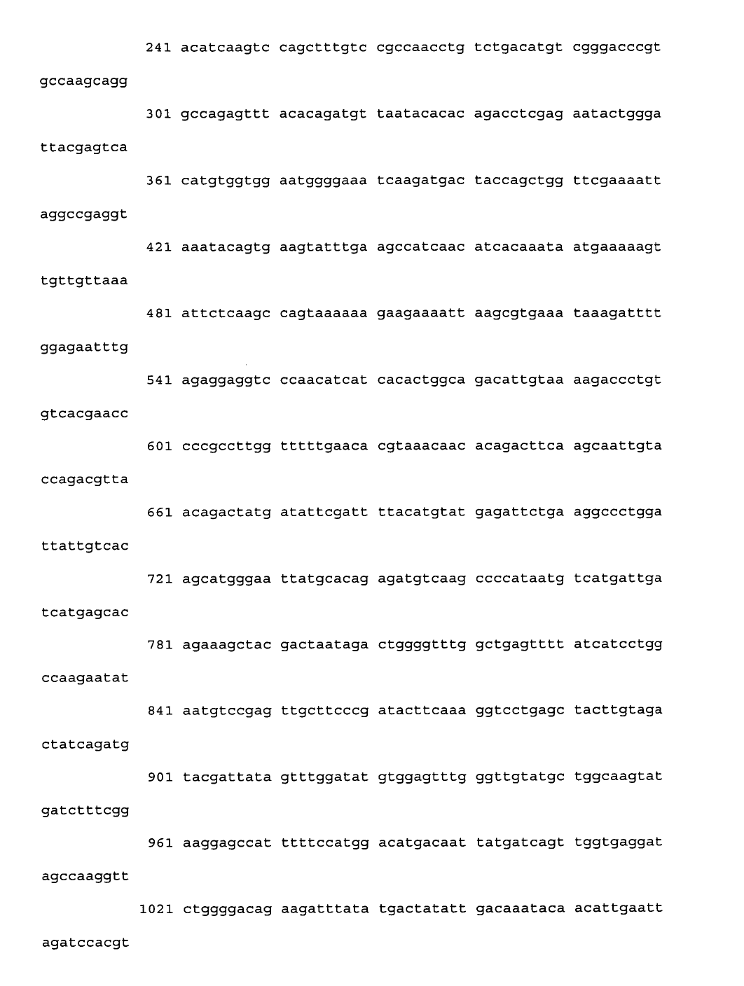

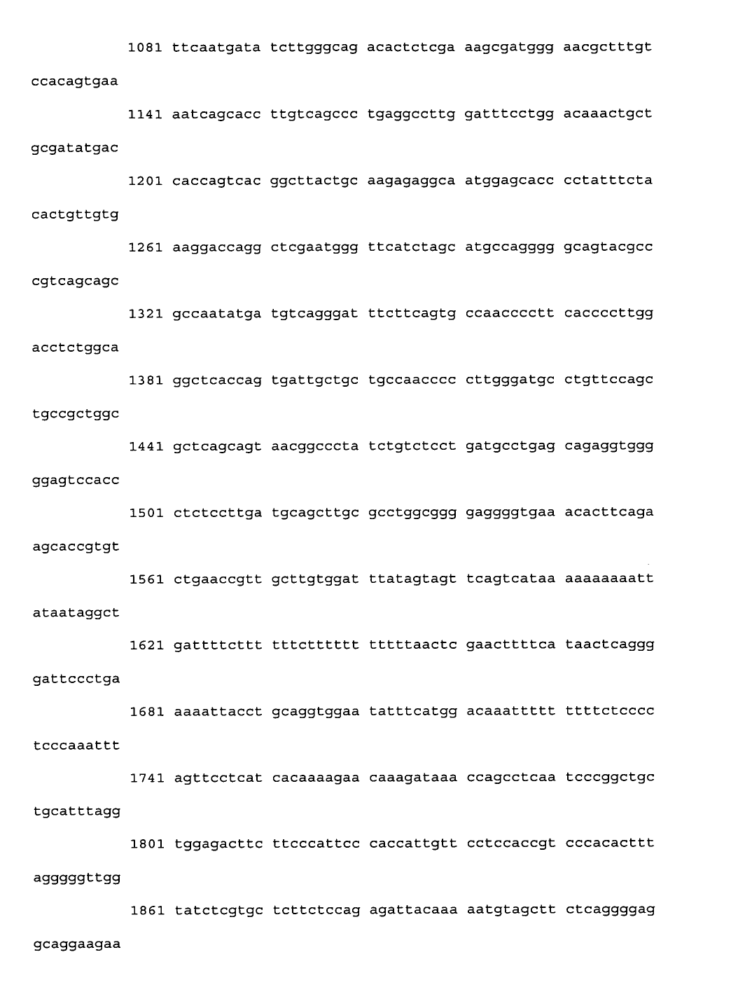

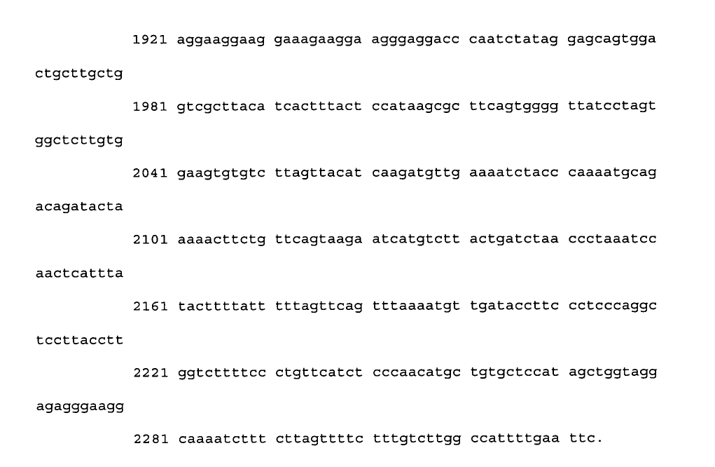

Given the disappearance of active carcinoma cells and the appearance of differentiated tissue in the residual tumor of the topically-treated mouse, the presence of histone deacytlase 1 (HDAC 1) was tested for using a polyclonal antibody and immunofluorescence microscopy in center sections from excised tumors (FIG. 8). FIG. 8 top row shows the same field of view of a section that received a topical application of nanoparticles. The left column shows HDAC staining and the right column shows bisbenzamide nuclear staining. The bottom row shows the same field of view of an intratumoral section. Low HDAC staining indicates a lack of cellular transcriptase activity.

In this analysis, higher levels of HDAC-1 indicate higher levels of transcriptional activity and low levels are consistent with a differentiated state (Vigushin & Coombs: 2002, Johnstone, R., Nature Rev. Drug Disc. (2002) 1:287-299). FIG. 8 shows that HDAC-1 signal levels are low in peripheral regions of the topically treated tumor and in a peripheral region bounded by the injection site and the tumor margin in the injected tumor. These data indicate two items, i) antisense to CK2α is able to induce differentiation and disappearance of carcinoma cells in vivo when enough drug can be delivered to the nuclei of carcinoma cells and ii) nanoparticles when injected intratumorally are capable of being “pumped out” by the pressure difference inherent in solid tumors due to their poor development of lymph vessels for drainage and pressure equalization. This indicates that nanoencapsulated compounds, including macromolecules, display the transport properties of small molecules. This is entirely consistent with the observed capacity to penetrate across endothelial and epidermal barriers in organ culture.

### Example 6

### Usefulness of the Entire Casein Kinase 2 Molecule for Anti-Tumor Treatment

Given the importance of Protein Kinase CK2 in regulating cell growth, its emerging role in regulating apoptosis suppression and differentiation, it was of interest to evaluate the usefulness of the entire sequence as a molecular target (Ahmed K. et. al, Trend Cell Biol (2002) 12(5): 226-30). CK2 sequences are available in public databases: e.g., Homo sapiens gene for casein kinase II alpha subunit, Accession X69951; Homo sapiens CKII beta associating protein mRNA, Accession AF475095; Homo sapiens CKII beta binding protein 2 mRNA, Accession AF412816; CSNK2A1=casein kinase II (CKII) human subunit alpha, Genomic, Accession S72393; H. sapiens CKII-alpha gene Accession X70251. Antisense sequences designed to other areas of the gene for the alpha subunit of the casein kinase 2 enzyme as well as the gene for beta subunit and the gene for alpha prime region were nanoencapsulated as before. Nanoencapsulated compounds were compared for anti-tumor activity by measuring the half-maximal dose level for inhibition of growth proliferation in Ca-9-22 tongue-derived squamous cell carcinoma cells. Results are documented in the following table:

TABLE 9

Utility of PKC CK2 genes and their sequences as
molecular targets for growth inhibition

| | | | | | |
|---|---|---|---|---|---|
| | | | | | IC₅₀ (%, |
| | | | | | Cisplatin |
| SEQ | | | Medicinal | | IC₅₀, |
| ID | Sequence | Parent | Chemistry | Cell | molar |
| NO | (5′ to 3′) | Gene | Format | Line | basis) |

| | | | | | |
|---|---|---|---|---|---|
| 1 | GTC CCG ACA TGT | CK2α (asCK2) | Published as | Ca- | 1-10% |
| | CAG ACA GG | | phosphodiester | 9-22 | 1-6% |
| | | | | SCC- | |
| | | | | 15 | |
| 2 | ccu guc uga cau guc | CK2α (RasCK2) | siRNA (chem. | SCC- | 7% |
| | ggg adtdt | | synthesized) | 15 | |
| 3 | atg tca gac agg ttg gcg | CK2α (MasCK2:) | morpholino | SCC- | 2% |
| | gac aaa g | | | 15 | |
| 4 | TCA CTG TAT Tta | CK2α (CR-1) | 3′BOH end- | Ca- | 11% |
| | cct cgg-butanol | | blocked | 9-22 | |
| | | | chimeric | | |
| 5 | GGA CCT CCT Ctc CK2c | CK2α (CR-2) | 3′BOH end- | Ca- | 11% |
| | aaa ttc tc-buoh | | blocked | 9-22 | |
| | | | chimeric | | |
| 6 | AGG ACC TTT Gaa | CK2α (CR-3) | 3′BOH end- | Ca- | 10% |
| | gta tcg gg-buoh | | blocked | 9-22 | |
| | | | chimeric | | |
| 7 | TGC TCC ATT Gcc | CK2α (CR-4) | 3′BOH end- | Ca- | 7% |
| | tct ctt gc-butanol | | blocked | 9-22 | |
| | | | chimeric | | |
| 8 | ggc atg gcg ggc ggg | CK2α′ (Prime-1) | 3′BOH end- | Ca- | 5.5% |
| | acc- buoh | | blocked | 9-22 | |
| | | | 2′OME | | |
| 9 | CGG GCA TGG C gg | CK2α′ (Prime-2) | 3′BOH end- | Ca- | 7.5% |
| | gcg gga cc - buoh | | blocked | 9-22 | |
| | | | chimeric | | |
| 10 | cat ctt cac gtc agc ggc- | CK2β (Beta-1) | 3′BOH end- | Ca- | 5.5% |
| | butanol | | blocked | 9-22 | |
| | | | 2′OME | | |
| 11 | CAT CTT CAC Gtc | CK2β(Beta-2) | 3′BOH end- | Ca- | 5.5% |
| | agc ggc tg-butanol | | blocked | 9-22 | |
| | | | chimeric | | |

Legend: RNA is small case-all
RNA is 2-O-methylated,
DNA is capitalized,
BOH is butanol

Based on the similarities in activity between the known region, which we have demonstrated convincing biological activity for and the previously unknown, but now discovered regions of the associated genes, we conclude that the entire and associated genes of the PKC CK2 (Casein Kinase 2) enzyme are valuable as a molecular target for drug discovery in disease states where proliferation or differentiation are deranged. This data also confirms the utility of nanoparticles for delivery of functional antisense by showing sequences from different genes.

### REFERENCES

Ahmed K. et. al, Trend Cell Biol (2002) 12(5): 226-30, “Joining the cell survival squad: an emerging role for Protein Kinase CK2.”
Aukhill I. et. al, J. Biol. Chem. (1993) 268(4):2542-2553, “Cell- and heparin-binding domains of the hexabrachion are identified by tenascin expression proteins.”
Bello et. al, Cancer Research (2001) 61: 8730-36, “Simultaneous inhibition of glioma angiogenesis, cell proliferation, and invasion by a naturally occurring fragment of human metalloproteinase 2.”
Faust, R., Tawfic, S., Davis, A., Bubash, L., and Ahmed, K.: Antisense oligonucleotides against PKCII-a inhibit growth of squamous cell carcinoma of the head and neck in vitro. Head & Neck 22: 341-346, 2000.
Glinsky et. al, Cancer Research (2001) 61:4851-57, “The role of Thomsen-Friedrich antigen in adhesion of human breast and prostate cancer cells to the endothelium.”
Hussain, N., Adv. Drug Deliv. Rev. (2000) 43:95-100, “Ligand-mediated tissue specific drug delivery.”
Jain et al., Sci. American (1994) 7:58-65, “Barriers to drug delivery in solid tumors.”
Koukoulis, G., Gould, v., Bhattacharyya, A., Howeedy, A., and Virtanen, I.: Tenascin in normal, reactive, hyperplastic and neoplastic tissues: biologic and pathologic implications. Hu. Pathol 22: 636-643, 1991
Jacob et. al, Cancer Research (1999) 59:4453-57, “Osteonectin promotes prostate cancer cell migration and invasion: a possiblee mechanism for metastasis to bone.”
Lee et. al, Crit. Rev. Ther. Drug Carr. Sys., (1997) 14:2 173-206, “Lipidic vector systems for gene transfer.”
Lackey et. al, 2002, Bioconjugate Chem. 13, 996-1001,” A biomimetic pH-responsive polymer directs endosomal release and intracellular delivery of an endocytosed antibody complex.”
Lakkaraju et. al, J. Biol. Chem. (2001) 276(34):32000-007, “Neurons are protected from excitotoxic death by p53 antisense oligonucleotides delivered in anionic liposomes.”
Livant et. al, Cancer Research (2000) 60: 309-20, “Anti-invasive, antitumorigenic, and antimetastatic activities of the PHSCN sequence in prostate carcinoma.”
Martin et. al, Int. J. Rad. Oncol. Biophys. (2000) 47(2): 277-90,” TGF-beta1 and radiation fibrosis: a master switch and a specific therapeutic target?”
Simpson et. al, J. Biol. Chem (2001) 276(21): 17949-57, “Hyaluronan synthase elevation in metastatic prostate carcinoma cells correlates with hyaluronan surface retention, a prerequisite for rapid adhesion to bone marrow endothelial cells.”
Tuxhorn et. al, J Urol. (2001) 166:2472-2483, “Reactive stroma in prostate tumor progression.”
Unger, G., Adams, G., Davis, A., Ahmed, K., (2002) “Effective chemotherapeutic activity by sub50-nm nanoparticle antisense to protein kinase CK2 for eradication of in vitro tumor nests via targeted caveolar-mediated endocytosis.”, AACR Proceedings, 43: 577.

The embodiments set forth herein are provided as examples, and are not intended to limit the scope or spirit of the invention. All patents, patent applications, publications and journal articles set forth herein are hereby incorporated herein by reference.

1. An antisense polynucleic acid comprising a sequence, wherein the antisense polynucleic acid suppresses the expression of a polypeptide encoded by a polynucleic acid sequence for the polypeptide chosen from the group consisting of SEQ ID NO 12 SEQ ID NO 13 and SEQ ID NO 14 and wherein the antisense polynucleic acid comprises a backbone that has at least two members of the group consisting of unmodified DNA/RNA, DNA/RNA with modified internucleoside linkages, 2′ modified RNA, p-ethoxy-2′omethyl RNA modification, 3′ end-blocked RNA, and 5′ end-blocked RNA.

2. The antisense oligonuclotide of claim 1 wherein the modified internucleoside linkages are selected form the group consisting of interphosphorothioate modifications, p-ethoxy modifications and morpholino modifications.

3. The antisense oligonucleotide of claim 1 wherein the antisense polynucleic acid has a number of residues that is at least 10.

4. The antisense oligonucleotide of claim 1 wherein the antisense polynucleic acid has a number of residues that ranges from 12 to 30.

5. The antisense oligonucleotide of claim 1 wherein the at least two members comprises two different internucleoside linkage modifications.

6. The antisense oligonucleotide of claim 1 wherein the at least two members comprises an internucleoside linkage modification and a 2′ modification.

7. A nonhuman mammal comprising the antisense polynucleic acid of claim 1.

8. A cell comprising the antisense polynucleic acid of claim 1.

9. A nanoparticle of less than about 100 nm that comprises the antisense polynucleic acid of claim 1.

10. A collection of particles comprising:
an agent, a surfactant molecule having an HLB value of less than about 6.0 units, a polymer soluble in aqueous solution, wherein the collection of particles has an average diameter of less than about 100 nanometers as measured by atomic force microscopy of a plurality of the particles following drying of the particles, wherein the agent comprises an antisense polynucleic acid that comprises a sequence, wherein the antisense polynucleic acid suppresses the expression of at least one member of the group consisting of protein kinase CK2, protein kinase CK2 alpha, and protein kinase CK2 beta, and wherein the antisense polynucleic acid comprises a backbone that has at least two members of the group consisting of unmodified DNA/RNA, DNA/RNA modified internucleoside linkages, 2′ modified RNA, p-ethoxy-2′omethyl RNA modification, 3′ end-blocked RNA, and 5′ end-blocked RNA.

11. The collection of particles of claim 10 wherein the antisense polynucleic acid has a number of residues that is at least 10.

12. The collection of particles of claim 10 wherein the antisense polynucleic acid has a number of residues that ranges from 12 to 30.

13. The collection of particles of claim 10 further comprising a cell recognition agent.

14. A nonhuman mammal comprising the collection of particles of claim 10.

15. A cell comprising the collection of particles of claim 10.

16. A collection of particles comprising a bioactive component, a surfactant molecule having an HLB value of less than about 6.0 units, and a biocompatible polymer, wherein the particle has an average diameter of less than about 200 nanometers as measured by atomic force microscopy of a plurality of the particles following drying of the particles, and wherein the bioactive component comprises an antisense polynucleic acid effective to inhibit expression of at least one member of the group consisting of protein kinase CK2, protein kinase CK2 alpha, and protein kinase CK2 beta, and the antisense comprising a backbone having at least two members of the group consisting of unmodified DNA/RNA, DNA/RNA with modified internucleoside linkages, 2′ modified RNA, p-ethoxy-2′omethyl RNA modification, 3′ end-blocked RNA, and 5′ end-blocked RNA.

17. A method of delivering a bioactive component to a cell or tissue comprising providing a collection of particles comprising an antisense molecule, a surfactant having an HLB value of less than about 6.0 units, and a biocompatible polymer, wherein the particle has an average diameter of less than about 200 nanometers as measured by atomic force microscopy of a plurality of the particles following drying of the particles, and wherein the bioactive component comprises an antisense polynucleic acid effective to inhibit expression of at least one member of the group consisting of protein kinase CK2, protein kinase CK2 alpha, and protein kinase CK2 beta, and the antisense comprising a backbone having at least two members of the group consisting of unmodified DNA/RNA, DNA/RNA with modified internucleoside linkages, 2′ modified RNA, p-ethoxy-2′omethyl RNA modification, 3′ end-blocked RNA, and 5′ end-blocked RNA.

18. The method of claim 16 wherein the cell is a member of the group consisting of glial cells, astrocytes, smooth muscle cells, myofibroblasts, vascular endothelial cells, leukaemic blasts, vascular endothelial cells in solid tumors, B-cell lymphoproliferative disease cells, acute myeloid leukemia cells, glial tumor cells, breast cancer cells, small-cell lung cancer cells, ovarian cancer cells, colorectal cancer cells, blood vessel medial cells, squamous cell carcinoma cells and epithelial-derived cancer cells.

19. A method of delivering an anti-cancer agent to cancer cells, the method comprising contacting the cancer cells with a collection of particles comprising the anticancer agents, a surfactant having an HLB value less than about 6.0 units, and a biocompatible polymer, wherein the anticancer agent comprises an antisense polynucleic acid effective to inhibit expression of at least one member of the group consisting of protein kinase CK2, protein kinase CK2 alpha, and protein kinase CK2 beta, and the antisense comprising a backbone having at least two members of the group consisting of unmodified DNA/RNA, DNA/RNA with modified internucleoside linkages, 2′ modified RNA, p-ethoxy-2′omethyl RNA modification, 3′ end-blocked RNA, and 5′ end-blocked RNA.

20. The method of claim 18 wherein the collection of particles further comprises a cell recognition component.
